SEQUENCE LISTING (69 sequences)

SEQ ID NO: 1 - protein, 181 aa, Aliatypus gulosus
GAASSSSTIITTKSASASAAADASAAATASAASRSSANAAASAFAQSFSSILLESGYFCSIFGSSISSSYAAAIASAASRAAAESNGYTTHAYACAKAVASAVERVTSGADAYAYAQAISDALSHALLYTGRLNTANANSLASAFAYAFANAAAQASASSASAGAASASGAASASGAGSAS

SEQ ID NO: 2 - protein, 126 aa, Plectreurys tristis
GAGAGAGAGAGAGAGAGSGASTSVSTSSSSGSGAGAGAGSGAGSGAGAGSGAGAGAGAGGAGAGFGSGLGLGYGVGLSSAQAQAQAQAAAQAQAQAQAQAYAAAQAQAQAQAQAQAAAAAAAAAAA

SEQ ID NO: 3 - protein, 239 aa, Plectreurys tristis
GAAQKQPSGESSVATASAAATSVTSGGAPVGKPGVPAPIFYPQGPLQQGPAPGPSNVQPGTSQQGPIGGVGGSNAFSSSFASALSLNRGFTEVISSASATAVASAFQKGLAPYGTAFALSAASAAADAYNSIGSGANAFAYAQAFARVLYPLVQQYGLSSSAKASAFASAIASSFSSGTSGQGPSIGQQQPPVTISAASASAGASAAAVGGGQVGQGPYGGQQQSTAASASAAAATATS

SEQ ID NO: 4 - protein, 182 aa, Araneus gemmoides
GNVGYQLGLKVANSLGLGNAQALASSLSQAVSAVGVGASSNAYANAVSNAVGQVLAGQGILNAANAGSLASSFASALSSSAASVASQSASQSQAASQSQAAASAFRQAASQSASQSDSRAGSQSSTKTTSTSTSGSQADSRSASSSASQASASAFAQQSSASLSSSSSFSSAFSSATSISAV

SEQ ID NO: 5 - protein, 180 aa, Argiope aurantia
GSLASSFASALSASAASVASSAAAQAASQSQAAASAFSRAASQSASQSAARSGAQSISTTTTTSTAGSQAASQSASSAASQASASSFARASSASLAASSSFSSAFSSANSLSALGNVGYQLGFNVANNLGIGNAAGLGNALSQAVSSVGVGASSSTYANAVSNAVGQFLAGQGILNAANA

SEQ ID NO: 6 - protein, 199 aa, Deinopis spinosa
GASASAYASAISNAVGPYLYGLGLFNQANAASFASSFASAVSSAVASASASAASSAYAQSAAAQAQAASSAFSQAAAQSAAAASAGASAGAGASAGAGAVAGAGAVAGAGAVAGASAAAASQAAASSSASAVASAFAQSASYALASSSAFANAFASATSAGYLGSLAYQLGLTTAYNLGLSNAQAFASTLSQAVTGVGL

SEQ ID NO: 7 - protein, 171 aa, Nephila clavipes
GATAASYGNALSTAAAQFFATAGLLNAGNASALASSFARAFSASAESQSFAQSQAFQQASAFQQAASRSASQSAAEAGSTSSSTTTTTSAARSQAASQSASSSYSSAFAQAASSSLATSSALSRAFSSVSSASAASSLAYSIGLSAARSLGIADAAGLAGVLARAAGALGQ

SEQ ID NO: 8 - protein, 268 aa, Argiope trifasciata
GGAPGGGPGGAGPGGAGFGPGGGAGFGPGGGAGFGPGGAAGGPGGPGGPGGPGGAGGYGPGGAGGYGPGGVGPGGAGGYGPGGAGGYGPGGSGPGGAGPGGAGGEGPVTVDVDVTVGPEGVGGGPGGAGPGGAGFGPGGGAGFGPGGAPGAPGGPGGPGGPGGPGGPGGVGPGGAGGYGPGGAGGVGPAGTGGFGPGGAGGFGPGGAGGFGPGGAGGFGPAGAGGYGPGGVGPGGAGGFGPGGVGPGGSGPGGAGGEGPVTVDVDVSV

SEQ ID NO: 9 - protein, 420 aa, Nephila clavipes
GVSYGPGGAGGPYGPGGPYGPGGEGPGGAGGPYGPGGVGPGGSGPGGYGPGGAGPGGYGPGGSGPGGYGPGGSGPGGYGPGGSGPGGYGPGGSGPGGYGPGGYGPGGSGPGGSGPGGSGPGGYGPGGTGPGGSGPGGYGPGGSGPGGSGPGGYGPGGSGPGGFGPGGSGPGGYGPGGSGPGGAGPGGVGPGGFGPGGAGPGGAAPGGAGPGGAGPGGAGPGGAGPGGAGPGGAGPGGAGGAGGAGGSGGAGGSGGTTIIEDLDITIDGADGPITISEELPISGAGGSGPGGAGPGGVGPGGSGPGGVGPGGSGPGGVGPGGSGPGGVGPGGAGGPYGPGGSGPGGAGGAGGPGGAYGPGGSYGPGGSGGPGGAGGPYGPGGEGPGGAGGPYGPGGAGGPYGPGGAGGPYGPGGEGGPYGP

SEQ ID NO: 10 - protein, 376 aa, Latrodectus hesperus
GINVDSDIGSVTSLILSGSTLQMTIPAGGDDLSGGYPGGFPAGAQPSGGAPVDFGGPSAGGDVAAKLARSLASTLASSGVFRAAFNSRVSTPVAVQLTDALVQKIASNLGLDYATASKLRKASQAVSKVRMGSDTNAYALAISSALAEVLSSSGKVADANINQIAPQLASGIVLGVSTTAPQFGVDLSSINVNLDISNVARNMQASIQGGPAPITAEGPDFGAGYPGGAPTDLSGLDMGAPSDGSRGGDATAKLLQALVPALLKSDVFRAIYKRGTRKQVVQYVTNSALQQAASSLGLDASTISQLQTKATQALSSVSADSDSTAYAKAFGLAIAQVLGTSGQVNDANVNQIGAKLATGILRGSSAVAPRLGIDLS

SEQ ID NO: 11 - protein, 200 aa, Argiope trifasciata
GAGYTGPSGPSTGPSGYPGPLGGGAPFGQSGFGGSAGPQGGFGATGGASAGLISRVANALANTSTLRTVLRTGVSQQIASSVVQRAAQSLASTLGVDGNNLARFAVQAVSRLPAGSDTSAYAQAFSSALFNAGVLNASNIDTLGSRVLSALLNGVSSAAQGLGINVDSGSVQSDISSSSSFLSTSSSSASYSQASASSTS

SEQ ID NO: 12 - protein, 357 aa, Uloborus diversus
GASAADIATAIAASVATSLQSNGVLTASNVSQLSNQLASYVSSGLSSTASSLGIQLGASLGAGFGASAGLSASTDISSSVEATSASTLSSSASSTSVVSSINAQLVPALAQTAVLNAAFSNINTQNAIRIAELLTQQVGRQYGLSGSDVATASSQIRSALYSVQQGSASSAYVSAIVGPLITALSSRGVVNASNSSQIASSLATAILQFTANVAPQFGISIPTSAVQSDLSTISQSLTAISSQTSSSVDSSTSAFGGISGPSGPSPYGPQPSGPTFGPGPSLSGLTGFTATFASSFKSTLASSTQFQLIAQSNLDVQTRSSLISKVLINALSSLGISASVASSIAASSSQSLLSVSA

SEQ ID NO: 13 - protein, 32 aa, Euprosthenops australis
GGQGGQGQGRYGQGAGSSAAAAAAAAAAAAAA

SEQ ID NO: 14 - protein, 42 aa, Tetragnatha kauaiensis
GGLGGGQGAGQGGQQGAGQGGYGSGLGGAGQGASAAAAAAAA

SEQ ID NO: 15 - protein, 42 aa, Argiope aurantia
GGYGPGAGQQGPGSQGPGSGGQQGPGGLGPYGPSAAAAAAAA

SEQ ID NO: 16 - protein, 46 aa, Deinopis spinosa
GPGGYGGPGQQGPGQGQYGPGTGQQGQGPSGQQGPAGAAAAAAAAA

SEQ ID NO: 17 - protein, 42 aa, Nephila clavata
GPGGYGLGQQGPGQQGPGQQGPAGYGPSGLSGPGGAAAAAAA

SEQ ID NO: 18 - protein, 174 aa, Deinopis spinosa
GAGYGAGAGAGGGAGAGTGYGGGAGYGTGSGAGYGAGVGYGAGAGAGGGAGAGAGGGTGAGAGGGAGAGYGAGTGYGAGAGAGGGAGAGAGAGAGAGAGAGSGAGAGYGAGAGYGAGAGAGGVAGAGAAGGAGAAGGAGAAGGAGAAGGAGAGAGAGSGAGAGAGGGARAGAGG

SEQ ID NO: 19 - protein, 149 aa, Latrodectus hesperus
GGGYGRGQGAGAGVGAGAGAAAGAAAIARAGGYGQGAGGYGQGQGAGAAAGAAAGAGAGGYGQGAGGYGRGQGAGAGAGAGAGARGYGQGAGAGAAAGAAASAGAGGYGQGAGGYGQGQGAGAAAGAAASAGAGGYGQGAGGYGQGQGA

SEQ ID NO: 20 - protein, 161 aa, Nephila clavipes
GAGAGGAGYGRGAGAGAGAAAGAGAGAAAGAGAGAGGYGGQGGYGAGAGAGAAAAAGAGAGGAAGYSRGGRAGAAGAGAGAAAGAGAGAGGYGGQGGYGAGAGAGAAAAAGAGSGGAGGYGRGAGAGAAAGAGAAAGAGAGAGGYGGQGGYGAGAGAAAAA

SEQ ID NO: 21 - protein, 186 aa, Nephilengys cruentata
GAGAGVGGAGGYGSGAGAGAGAGAGAASGAAAGAAAGAGAGGAGGYGTGQGYGAGAGAGAGAGAGGAGGYGRGAGAGAGAGAGGAGGYGAGQGYGAGAGAGAAAAAGDGAGAGGAGGYGRGAGAGAGAGAAAGAGAGGAGGYGAGQGYGAGAGAGAAAGAGAGGAGGYGAGQGYGAGAGAGAAAAA

SEQ ID NO: 22 - protein, 132 aa, Uloborus diversus
GSGAGAGSGYGAGAGAGAGSGYGAGSSASAGSAINTQTVTSSTTTSSQSSAAATGAGYGTGAGTGASAGAAASGAGAGYGGQAGYGQGAGASARAAGSGYGAGAGAAAAAGSGYGAGAGAGAGSGYGAGAAA

SEQ ID NO: 23 - protein, 198 aa, Uloborus diversus
GAGAGYRGQAGYIQGAGASAGAAAAGAGVGYGGQAGYGQGAGASAGAAAAAGAGAGRQAGYGQGAGASAGAAAAGAGAGRQAGYGQGAGASAGAAAAGADAGYGGQAGYGQGAGASAGAAASGAGAGYGGQAGYGQGAGASAGAAAAGAGAGYLGQAGYGQGAGASAGAAAGAGAGYGGQAGYGQGTGAAASAAASSA

SEQ ID NO: 24 - protein, 190 aa, Araneus ventricosus
GGQGGQGGYGGLGSQGAGQGGYGAGQGAAAAAAAAGGAGGAGRGGLGAGGAGQGYGAGLGGQGGAGQAAAAAAAGGAGGARQGGLGAGGAGQGYGAGLGGQGGAGQGGAAAAAAAAGGQGGQGGYGGLGSQGAGQGGYGAGQGGAAAAAAAAGGQGGQGGYGGLGSQGAGQGGYGGRQGGAGAAAAAAAA

SEQ ID NO: 25 - protein, 166 aa, Dolomedes tenebrosus
GGAGAGQGSYGGQGGYGQGGAGAATATAAAAGGAGSGQGGYGGQGGLGGYGQGAGAGAAAAAAAAAGGAGAGQGGYGGQGGQGGYGQGAGAGAAAAAAGGAGAGQGGYGGQGGYGQGGGAGAAAAAAAASGGSGSGQGGYGGQGGLGGYGQGAGAGAGAAASAAAA

SEQ ID NO: 26 - protein, 177 aa, Nephilengys cruentata
GGAGQGGYGGLGGQGAGAAAAAAGGAGQGGYGGQGAGQGAAAAAASGAGQGGYEGPGAGQGAGAAAAAAGGAGQGGYGGLGGQGAGQGAGAAAAAAGGAGQGGYGGLGGQGAGQGAGAAAAAAGGAGQGGYGGQGAGQGAAAAAAGGAGQGGYGGLGSGQGGYGRQGAGAAAAAAAA

SEQ ID NO: 27 - protein, 174 aa, Nephilengys cruentata
GGAGQGGYGGLGGQGAGAAAAAAGGAGQGGYGGQGAGQGAAAAAASGAGQGGYGGPGAGQGAGAAAAAAGGAGQGGYGGLGGQGAGQGAGAAAAAAGGAGQGGYGGQGAGQGAAAAAAGGAGQGGYGGLGSGQGGYGGQGAGAAAAAGGAGQGGYGGLGGQGAGQGAGAAAAAA

SEQ ID NO: 28 - protein, 22 aa, Saccharomyces cerevisiae
MFSLKALLPLALLLVSANQVAA

SEQ ID NO: 29 - protein, 23 aa, Pichia pastoris
MSFSSNVPQLFLLLVLLTNIVSG

SEQ ID NO: 30 - protein, 20 aa, Pichia pastoris
MKLSTNLILAIAAASAVVSA

SEQ ID NO: 31 - protein, 18 aa, Gallus gallus
MRSLLILVLCFLPLAALG

SEQ ID NO: 32 - skipped

SEQ ID NO: 33 - protein, 89 aa, Artificial Sequence
  Description of Artificial Sequence: Synthetic polypeptide
MRFPSIFTAVLFAASSALAAPVNTTTEDETAQIPAEAVIGYLDLEGDFDVAVLPFSNSTNNGLLFINTTIASIAAKEEGVSLDKREAEA

SEQ ID NO: 34 - protein, 89 aa, Artificial Sequence
  Description of Artificial Sequence: Synthetic polypeptide
MRFPSIFTAVLFAASSALAAPVNTTTEDETAQIPAEAVIGYSDLEGDFDVAVLPFSNSTNNGLLFINTTIASIAAKEEGVSLEKREAEA

SEQ ID NO: 35 - protein, 92 aa, Artificial Sequence
  Description of Artificial Sequence: Synthetic polypeptide
MFSLKALLPLALLLVSANQVAAAPVNTTTEDETAQIPAEAVIGYSDLEGDFDVAVLPFSNSTNNGLLFINTTIASIAAKEEGVSLEKREAEA

SEQ ID NO: 36 - protein, 93 aa, Artificial Sequence
  Description of Artificial Sequence: Synthetic polypeptide
MSFSSNVPQLFLLLVLLTNIVSGAPVNTTTEDETAQIPAEAVIGYSDLEGDFDVAVLPFSNSTNNGLLFINTTIASIAAKEEGVSLEKREAEA

SEQ ID NO: 37 - protein, 90 aa, Artificial Sequence
  Description of Artificial Sequence: Synthetic polypeptide
MKLSTNLILAIAAASAVVSAAPVNTTTEDETAQIPAEAVIGYSDLEGDFDVAVLPFSNSTNNGLLFINTTIASIAAKEEGVSLEKREAEA

SEQ ID NO: 38 - protein, 88 aa, Artificial Sequence
  Description of Artificial Sequence: Synthetic polypeptide
MRSLLILVLCFLPLAALGAPVNTTTEDETAQIPAEAVIGYSDLEGDFDVAVLPFSNSTNNGLLFINTTIASIAAKEEGVSLEKREAEA

SEQ ID NO: 39 - protein, 1416 aa, Artificial Sequence
  Description of Artificial Sequence: Synthetic polypeptide
GGYGPGQRAGPGQGAGPGQGVGPGQGVGTGGQGTGASSAAAASAGTSGYGPGVSGYGPAQGAGPGGQGTGASSAAATSAGTSGYGPGYPGYGQGPASGPTADKYGPGIGRYAPGRSTTSTSAATASATTVNNGPQIGGYGPGQGIGPAATSAPGASGYGPGVSGYGPGQGAGPGQGAGPWQGVGPGQGAGPGGQGSGASSEAAASAGTSAYGPGVSGYGPGQGAGPGGQGSGASSRAAASAGTRGYGPGYPGYGQGPASGPTAAYKYGPGIGGYAPGRSTTSTSAATASATTVDIGPQVGGYGPGQGIAPAAAAPGSSGYGPGVSGYGPGQGAGPGGQGTGASSAAAASAGTSGYGSGVSGYGPGQGAGPGQGAGPGQGVGPGQGAGLGQGVGPGQGAGPGGQGSGASTAAAASAGTSGYAPGVSGYGPGQGAGPGQGAGPGQGAGPGQGVGPGQGAGPGGQGSGASSAAAAGGYGPGQRAGPGQGAGPGQGVGPGQGVGTGGQGTGASSAAAASAGTSGYGPGVSGYGPAQGAGPGGQGTGASSAAATSAGTSGYGPGYPGYGQGPASGPTADKYGPGIGRYAPGRSTTSTSAATASATTVNNGPQIGGYGPGQGIGPAATSAPGASGYGPGVSGYGPGQGAGPGQGAGPWQGVGPGQGAGPGGQGSGASSEAAASAGTSAYGPGVSGYGPGQGAGPGGQGSGASSRAAASAGTRGYGPGYPGYGQGPASGPTAAYKYGPGIGGYAPGRSTTSTSAATASATTVDIGPQVGGYGPGQGIAPAAAAPGSSGYGPGVSGYGPGQGAGPGGQGTGASSAAAASAGTSGYGSGVSGYGPGQGAGPGQGAGPGQGVGPGQGAGLGQGVGPGQGAGPGGQGSGASTAAAASAGTSGYAPGVSGYGPGQGAGPGQGAGPGQGAGPGQGVGPGQGAGPGGQGSGASSAAAAGGYGPGQRAGPGQGAGPGQGVGPGQGVGTGGQGTGASSAAAASAGTSGYGPGVSGYGPAQGAGPGGQGTGASSAAATSAGTSGYGPGYPGYGQGPASGPTADKYGPGIGRYAPGRSTTSTSAATASATTVNNGPQIGGYGPGQGIGPAATSAPGASGYGPGVSGYGPGQGAGPGQGAGPWQGVGPGQGAGPGGQGSGASSEAAASAGTSAYGPGVSGYGPGQGAGPGGQGSGASSRAAASAGTRGYGPGYPGYGQGPASGPTAAYKYGPGIGGYAPGRSTTSTSAATASATTVDIGPQVGGYGPGQGIAPAAAAPGSSGYGPGVSGYGPGQGAGPGGQGTGASSAAAASAGTSGYGSGVSGYGPGQGAGPGQGAGPGQGVGPGQGAGLGQGVGPGQGAGPGGQGSGASTAAAASAGTSGYAPGVSGYGPGQGAGPGQGAGPGQGAGPGQGVGPGQGAGPGGQGSGASSAAAA

SEQ ID NO: 40 - protein, 840 aa, Artificial Sequence
  Description of Artificial Sequence: Synthetic polypeptide
GGYTQRQNEVITTVSTTRQKTADYGQKQVSGASAAVSTSSAGGYTQGPGGYGPGQGAVAGGYGPGAGSYGAGAIDASGGYGQGAGTAAGASASAGAGAATGVGPGGYGQGLGGYGQSAGQGAGGYRQGAGTAAGASASAGAGAATGVGPGGYGQGLGGYGQAAGQGAGGYGQGAGTATSTATGAGTGGYGRLAGGYGQGAGGYGQAAAGAAADATAGAGGYDRATGAFGPSTRRAAGGSGLGAGTAPGAFSGSGAGGKGPGDYGSSQGASASSSAAAAASGGYTQRQNEVITTVSTTRQKTADYGQKQVSGASAAVSTSSAGGYTQGPGGYGPGQGAVAGGYGPGAGSYGAGAIDASGGYGQGAGTAAGASASAGAGAATGVGPGGYGQGLGGYGQSAGQGAGGYRQGAGTAAGASASAGAGAATGVGPGGYGQGLGGYGQAAGQGAGGYGQGAGTATSTATGAGTGGYGRLAGGYGQGAGGYGQAAAGAAADATAGAGGYDRATGAFGPSTRRAAGGSGLGAGTAPGAFSGSGAGGKGPGDYGSSQGASASSSAAAAASGGYTQRQNEVITTVSTTRQKTADYGQKQVSGASAAVSTSSAGGYTQGPGGYGPGQGAVAGGYGPGAGSYGAGAIDASGGYGQGAGTAAGASASAGAGAATGVGPGGYGQGLGGYGQSAGQGAGGYRQGAGTAAGASASAGAGAATGVGPGGYGQGLGGYGQAAGQGAGGYGQGAGTATSTATGAGTGGYGRLAGGYGQGAGGYGQAAAGAAADATAGAGGYDRATGAFGPSTRRAAGGSGLGAGTAPGAFSGSGAGGKGPGDYGSSQGASASSSAAAAAS

SEQ ID NO: 41 - protein, 840 aa, Artificial Sequence; identical to SEQ ID NO: 40
  Description of Artificial Sequence: Synthetic polypeptide

SEQ ID NO: 42 - protein, 885 aa, Artificial Sequence
  Description of Artificial Sequence: Synthetic polypeptide
GGAGQGGYGGLGGQGAGAAAAAAGGAGQGGYGGQGAGQGAAAAAASGAGQGGYEGPGAGQGAGAAAAAAGGAGQGGYGGLGGQGAGQGAGAAAAAAGGAGQGGYGGLGGQGAGQGAGAAAAAAGGAGQGGYGGQGAGQGAAAAAAGGAGQGGYGGLGSGQGGYGRQGAGAAAAAAAAGGAGQGGYGGLGGQGAGAAAAAAGGAGQGGYGGQGAGQGAAAAAASGAGQGGYEGPGAGQGAGAAAAAAGGAGQGGYGGLGGQGAGQGAGAAAAAAGGAGQGGYGGLGGQGAGQGAGAAAAAAGGAGQGGYGGQGAGQGAAAAAAGGAGQGGYGGLGSGQGGYGRQGAGAAAAAAAAGGAGQGGYGGLGGQGAGAAAAAAGGAGQGGYGGQGAGQGAAAAAASGAGQGGYEGPGAGQGAGAAAAAAGGAGQGGYGGLGGQGAGQGAGAAAAAAGGAGQGGYGGLGGQGAGQGAGAAAAAAGGAGQGGYGGQGAGQGAAAAAAGGAGQGGYGGLGSGQGGYGRQGAGAAAAAAAAGGAGQGGYGGLGGQGAGAAAAAAGGAGQGGYGGQGAGQGAAAAAASGAGQGGYEGPGAGQGAGAAAAAAGGAGQGGYGGLGGQGAGQGAGAAAAAAGGAGQGGYGGLGGQGAGQGAGAAAAAAGGAGQGGYGGQGAGQGAAAAAAGGAGQGGYGGLGSGQGGYGRQGAGAAAAAAAAGGAGQGGYGGLGGQGAGAAAAAAGGAGQGGYGGQGAGQGAAAAAASGAGQGGYEGPGAGQGAGAAAAAAGGAGQGGYGGLGGQGAGQGAGAAAAAAGGAGQGGYGGLGGQGAGQGAGAAAAAAGGAGQGGYGGQGAGQGAAAAAAGGAGQGGYGGLGSGQGGYGRQGAGAAAAAAAA

SEQ ID NO: 43 - protein, 996 aa, Artificial Sequence
  Description of Artificial Sequence: Synthetic polypeptide
GGYGSGASARAAAGAGGNSEQGGYGAGAGAAATAGSGAGGAGSYRRGSGAEATAGAGAGSAGGYGGQGGYGAGAGADAGSAGDYGRGAGAGAGAEAGTSSAGGYGEQGGYGTGAAAAAGAGAGRAGGYGRGSGGAGGYGRPGAAGADGAGGYGGQGGYDAGAGAGAGGYGSGASARAAAGAGGNSEQGGYGAGAGAAATAGSGAGGAGSYRRGSGAEATAGAGAGSAGGYGGQGGYGAGAGADAGSAGDYGRGAGAGAGAEAGTSSAGGYGEQGGYGTGAAAAAGAGAGRAGGYGRGSGGAGGYGRPGAAGADGAGGYGGQGGYDAGAGAGAGGYGSGASARAAAGAGGNSEQGGYGAGAGAAATAGSGAGGAGSYRRGSGAEATAGAGAGSAGGYGGQGGYGAGAGADAGSAGDYGRGAGAGAGAEAGTSSAGGYGEQGGYGTGAAAAAGAGAGRAGGYGRGSGGAGGYGRPGAAGADGAGGYGGQGGYDAGAGAGAGGYGSGASARAAAGAGGNSEQGGYGAGAGAAATAGSGAGGAGSYRRGSGAEATAGAGAGSAGGYGGQGGYGAGAGADAGSAGDYGRGAGAGAGAEAGTSSAGGYGEQGGYGTGAAAAAGAGAGRAGGYGRGSGGAGGYGRPGAAGADGAGGYGGQGGYDAGAGAGAGGYGSGASARAAAGAGGNSEQGGYGAGAGAAATAGSGAGGAGSYRRGSGAEATAGAGAGSAGGYGGQGGYGAGAGADAGSAGDYGRGAGAGAGAEAGTSSAGGYGEQGGYGTGAAAAAGAGAGRAGGYGRGSGGAGGYGRPGAAGADGAGGYGGQGGYDAGAGAGAGGYGSGASARAAAGAGGNSEQGGYGAGAGAAATAGSGAGGAGSYRRGSGAEATAGAGAGSAGGYGGQGGYGAGAGADAGSAGDYGRGAGAGAGAEAGTSSAGGYGEQGGYGTGAAAAAGAGAGRAGGYGRGSGGAGGYGRPGAAGADGAGGYGGQGGYDAGAGAGA

SEQ ID NO: 44 - protein, 1104 aa, Artificial Sequence
  Description of Artificial Sequence: Synthetic polypeptide
GGYGQGSGGYGQNAGAAAGSGANGQGAGGYGQGAAAVAAAGAGAGGYGQGAGGYGQDAGGYGQGAGGNGQGVVDAAGYGPGSQGYGQSAAATSSAAAGASATGYTERQNEVVTTVTTTRQETADRRQAARASAAVSTSAAAGYGQGTRGYGQVPGAAAGAGGYGQGAGGYGQGAAVGSSAGSGVAGYGQGSGGYGQSAAAAAGAGAYGQGAGGYGQGAGAATGSGAGGCGQGAGGYGQDAGAAAGAYGQGAGGYGQGAASGVATGTGAGGYGQGAGGYGQGASATAVAAAGAGAGIIGQGAGVYGQSAVSAAGAAGDTGAGGYGQSTGGYGPGSGAGAGAAAGAGGYGPGSQGYGQGAASTSSAAAGAGGYGQGSGGYGQNAGAAAGSGANGQGAGGYGQGAAAVAAAGAGAGGYGQGAGGYGQDAGGYGQGAGGNGQGVVDAAGYGPGSQGYGQSAAATSSAAAGASATGYTERQNEVVTTVTTTRQETADRRQAARASAAVSTSAAAGYGQGTRGYGQVPGAAAGAGGYGQGAGGYGQGAAVGSSAGSGVAGYGQGSGGYGQSAAAAAGAGAYGQGAGGYGQGAGAATGSGAGGCGQGAGGYGQDAGAAAGAYGQGAGGYGQGAASGVATGTGAGGYGQGAGGYGQGASATAVAAAGAGAGIIGQGAGVYGQSAVSAAGAAGDTGAGGYGQSTGGYGPGSGAGAGAAAGAGGYGPGSQGYGQGAASTSSAAAGAGGYGQGSGGYGQNAGAAAGSGANGQGAGGYGQGAAAVAAAGAGAGGYGQGAGGYGQDAGGYGQGAGGNGQGVVDAAGYGPGSQGYGQSAAATSSAAAGASATGYTERQNEVVTTVTTTRQETADRRQAARASAAVSTSAAAGYGQGTRGYGQVPGAAAGAGGYGQGAGGYGQGAAVGSSAGSGVAGYGQGSGGYGQSAAAAAGAGAYGQGAGGYGQGAGAATGSGAGGCGQGAGGYGQDAGAAAGAYGQGAGGYGQGAASGVATGTGAGGYGQGAGGYGQGASATAVAAAGAGAGIIGQGAGVYGQSAVSAAGAAGDTGAGGYGQSTGGYGPGSGAGAGAAAGAGGYGPGSQGYGQGAASTSSAAAGA

SEQ ID NO: 45 - protein, 1104 aa, Artificial Sequence; identical to SEQ ID NO: 44
  Description of Artificial Sequence: Synthetic polypeptide

SEQ ID NO: 46 - protein, 930 aa, Artificial Sequence
  Description of Artificial Sequence: Synthetic polypeptide
GGYTQKQNEVITTVSTTRQKIADYGQKQASGASAAVSTSSAGGYAQGPGGYGPGKGAGATTGAGARGYSQGPGGYAQGVSTAAGAAIAGAGGYGPSTGPYGQGAIDASGGYGQGVGTAAGASASAGSGAATGVGPVGYGQGLGGYGQAVGQGAGGYGQGAGAATVTVTAAVPGGYGPGAGGYGQGVGAAAGAGTDAGIGGYGQGAGGFGQGGAAASAATGAGPGGYGLGAGGYGQPIGATAGATAGAGGYGQGAGVSGAGSRGAPAGYGPGAGPAAGATSGAVAGGKGPGGYGPSQVASASSSAAAAAASGGYTQKQNEVITTVSTTRQKIADYGQKQASGASAAVSTSSAGGYAQGPGGYGPGKGAGATTGAGARGYSQGPGGYAQGVSTAAGAAIAGAGGYGPSTGPYGQGAIDASGGYGQGVGTAAGASASAGSGAATGVGPVGYGQGLGGYGQAVGQGAGGYGQGAGAATVTVTAAVPGGYGPGAGGYGQGVGAAAGAGTDAGIGGYGQGAGGFGQGGAAASAATGAGPGGYGLGAGGYGQPIGATAGATAGAGGYGQGAGVSGAGSRGAPAGYGPGAGPAAGATSGAVAGGKGPGGYGPSQVASASSSAAAAAASGGYTQKQNEVITTVSTTRQKIADYGQKQASGASAAVSTSSAGGYAQGPGGYGPGKGAGATTGAGARGYSQGPGGYAQGVSTAAGAAIAGAGGYGPSTGPYGQGAIDASGGYGQGVGTAAGASASAGSGAATGVGPVGYGQGLGGYGQAVGQGAGGYGQGAGAATVTVTAAVPGGYGPGAGGYGQGVGAAAGAGTDAGIGGYGQGAGGFGQGGAAASAATGAGPGGYGLGAGGYGQPIGATAGATAGAGGYGQGAGVSGAGSRGAPAGYGPGAGPAAGATSGAVAGGKGPGGYGPSQVASASSSAAAAAAS

SEQ ID NO: 47 - protein, 930 aa, Artificial Sequence; identical to SEQ ID NO: 46
  Description of Artificial Sequence: Synthetic polypeptide

SEQ ID NO: 48 - protein, 740 aa, Artificial Sequence
  Description of Artificial Sequence: Synthetic polypeptide
GGYGRGQGAGAGVGAGAGAAAGAAAIARAGGYGQGAGGYGQGQGAGAAAGAAAGAGAGGYGQGAGGYGRGQGAGAGAGAGAGARGYGQGAGAGAAAGAAASAGAGGYGQGAGGYGQGQGAGAAAGAAASAGAGGYGQGAGGYGQGQGAGGYGRGQGAGAGVGAGAGAAAGAAAIARAGGYGQGAGGYGQGQGAGAAAGAAAGAGAGGYGQGAGGYGRGQGAGAGAGAGAGARGYGQGAGAGAAAGAAASAGAGGYGQGAGGYGQGQGAGAAAGAAASAGAGGYGQGAGGYGQGQGAGGYGRGQGAGAGVGAGAGAAAGAAAIARAGGYGQGAGGYGQGQGAGAAAGAAAGAGAGGYGQGAGGYGRGQGAGAGAGAGAGARGYGQGAGAGAAAGAAASAGAGGYGQGAGGYGQGQGAGAAAGAAASAGAGGYGQGAGGYGQGQGAGGYGRGQGAGAGVGAGAGAAAGAAAIARAGGYGQGAGGYGQGQGAGAAAGAAAGAGAGGYGQGAGGYGRGQGAGAGAGAGAGARGYGQGAGAGAAAGAAASAGAGGYGQGAGGYGQGQGAGAAAGAAASAGAGGYGQGAGGYGQGQGAGGYGRGQGAGAGVGAGAGAAAGAAAIARAGGYGQGAGGYGQGQGAGAAAGAAAGAGAGGYGQGAGGYGRGQGAGAGAGAGAGARGYGQGAGAGAAAGAAASAGAGGYGQGAGGYGQGQGAGAAAGAAASAGAGGYGQGAGGYGQGQGA

SEQ ID NO: 49 - protein, 972 aa, Artificial Sequence
  Description of Artificial Sequence: Synthetic polypeptide
GGYGQGAGGYGQGAGAAAGAAAGAGAGGYGRGAGSAAGAAAGAGVGEYGQGAGGYGQGAGAAAGAAAGAGAGGYGQGAGGYGQGAGGYGQGAGAAAGAGAGSYGQGAGGYGQGAGAAAGAAAGAGAGGYGQGAGGYGQGAGAAAGAAAGAGAGGYGQGAGGYGQGAGAAAGAGAGGYGRGAGSAAGAAAGSGAGGYGQGAGGYGQGAGAGAGGYGQGAGASTGAAAGAGAGGYGQGAGGYGQGSGAAAGAGGYGQGSQGYEQGAAATSSAAAGASSTGYTERQNEVVTTVTTTRQEIADRRQAASASGAVSTSAAAGYGQGAGTGGYGQGAGGYGQGAGAAAGAAAGAGAGGYGRGAGSAAGAAAGAGVGEYGQGAGGYGQGAGAAAGAAAGAGAGGYGQGAGGYGQGAGGYGQGAGAAAGAGAGSYGQGAGGYGQGAGAAAGAAAGAGAGGYGQGAGGYGQGAGAAAGAAAGAGAGGYGQGAGGYGQGAGAAAGAGAGGYGRGAGSAAGAAAGSGAGGYGQGAGGYGQGAGAGAGGYGQGAGASTGAAAGAGAGGYGQGAGGYGQGSGAAAGAGGYGQGSQGYEQGAAATSSAAAGASSTGYTERQNEVVTTVTTTRQEIADRRQAASASGAVSTSAAAGYGQGAGTGGYGQGAGGYGQGAGAAAGAAAGAGAGGYGRGAGSAAGAAAGAGVGEYGQGAGGYGQGAGAAAGAAAGAGAGGYGQGAGGYGQGAGGYGQGAGAAAGAGAGSYGQGAGGYGQGAGAAAGAAAGAGAGGYGQGAGGYGQGAGAAAGAAAGAGAGGYGQGAGGYGQGAGAAAGAGAGGYGRGAGSAAGAAAGSGAGGYGQGAGGYGQGAGAGAGGYGQGAGASTGAAAGAGAGGYGQGAGGYGQGSGAAAGAGGYGQGSQGYEQGAAATSSAAAGASSTGYTERQNEVVTTVTTTRQEIADRRQAASASGAVSTSAAAGYGQGAGT

SEQ ID NO: 50 - protein, 972 aa, Artificial Sequence; identical to SEQ ID NO: 49
  Description of Artificial Sequence: Synthetic polypeptide

SEQ ID NO: 51 - protein, 930 aa, Artificial Sequence
  Description of Artificial Sequence: Synthetic polypeptide
GAGAGVGGAGGYGSGAGAGAGAGAGAASGAAAGAAAGAGAGGAGGYGTGQGYGAGAGAGAGAGAGGAGGYGRGAGAGAGAGAGGAGGYGAGQGYGAGAGAGAAAAAGDGAGAGGAGGYGRGAGAGAGAGAAAGAGAGGAGGYGAGQGYGAGAGAGAAAGAGAGGAGGYGAGQGYGAGAGAGAAAAAGAGAGVGGAGGYGSGAGAGAGAGAGAASGAAAGAAAGAGAGGAGGYGTGQGYGAGAGAGAGAGAGGAGGYGRGAGAGAGAGAGGAGGYGAGQGYGAGAGAGAAAAAGDGAGAGGAGGYGRGAGAGAGAGAAAGAGAGGAGGYGAGQGYGAGAGAGAAAGAGAGGAGGYGAGQGYGAGAGAGAAAAAGAGAGVGGAGGYGSGAGAGAGAGAGAASGAAAGAAAGAGAGGAGGYGTGQGYGAGAGAGAGAGAGGAGGYGRGAGAGAGAGAGGAGGYGAGQGYGAGAGAGAAAAAGDGAGAGGAGGYGRGAGAGAGAGAAAGAGAGGAGGYGAGQGYGAGAGAGAAAGAGAGGAGGYGAGQGYGAGAGAGAAAAAGAGAGVGGAGGYGSGAGAGAGAGAGAASGAAAGAAAGAGAGGAGGYGTGQGYGAGAGAGAGAGAGGAGGYGRGAGAGAGAGAGGAGGYGAGQGYGAGAGAGAAAAAGDGAGAGGAGGYGRGAGAGAGAGAAAGAGAGGAGGYGAGQGYGAGAGAGAAAGAGAGGAGGYGAGQGYGAGAGAGAAAAAGAGAGVGGAGGYGSGAGAGAGAGAGAASGAAAGAAAGAGAGGAGGYGTGQGYGAGAGAGAGAGAGGAGGYGRGAGAGAGAGAGGAGGYGAGQGYGAGAGAGAAAAAGDGAGAGGAGGYGRGAGAGAGAGAAAGAGAGGAGGYGAGQGYGAGAGAGAAAGAGAGGAGGYGAGQGYGAGAGAGAAAAA

SEQ ID NO: 52 - protein, 930 aa, Artificial Sequence
  Description of Artificial Sequence: Synthetic polypeptide
GGYGQGAGAGAGAGAGAGAGAAAGAGAGGYGQGAGGYGRGQGAAAAAGAGAGGYGQGAGAGAGAGAGAAAGAGAGGYGQGAGGYGKGQGAAAAAFAGAGGYGQGAGAGAGAYAGAGAGAVAGAAAGAGAGGYGQGAGGYGRGQGAAAAGAGAGAGGYGQGAGAGAGAAANAGAGGYGQGAGGYGRGQGAAAAAGAGAGAGGYGQGAGAGTGAAAGAGASAGAGVGAGAGAAAGAAAGAGAGGYGQGAGGYGPGQGAAAAAGAGAGAGGYGQGSGAGAGAGAGAAAGAGAGGYGQGAGGYGRGQGAAAAGAGGYGQGAGAGAGAGAGAGAGAAAGAGAGGYGQGAGGYGRGQGAAAAAGAGAGGYGQGAGAGAGAGAGAAAGAGAGGYGQGAGGYGKGQGAAAAAFAGAGGYGQGAGAGAGAYAGAGAGAVAGAAAGAGAGGYGQGAGGYGRGQGAAAAGAGAGAGGYGQGAGAGAGAAANAGAGGYGQGAGGYGRGQGAAAAAGAGAGAGGYGQGAGAGTGAAAGAGASAGAGVGAGAGAAAGAAAGAGAGGYGQGAGGYGPGQGAAAAAGAGAGAGGYGQGSGAGAGAGAGAAAGAGAGGYGQGAGGYGRGQGAAAAGAGGYGQGAGAGAGAGAGAGAGAAAGAGAGGYGQGAGGYGRGQGAAAAAGAGAGGYGQGAGAGAGAGAGAAAGAGAGGYGQGAGGYGKGQGAAAAAFAGAGGYGQGAGAGAGAYAGAGAGAVAGAAAGAGAGGYGQGAGGYGRGQGAAAAGAGAGAGGYGQGAGAGAGAAANAGAGGYGQGAGGYGRGQGAAAAAGAGAGAGGYGQGAGAGTGAAAGAGASAGAGVGAGAGAAAGAAAGAGAGGYGQGAGGYGPGQGAAAAAGAGAGAGGYGQGSGAGAGAGAGAAAGAGAGGYGQGAGGYGRGQGAAAAGA

SEQ ID NO: 53 - protein, 396 aa, Artificial Sequence
  Description of Artificial Sequence: Synthetic polypeptide
GAGAGYRGQAGYIQGAGASAGAAAAGAGVGYGGQAGYGQGAGASAGAAAAAGAGAGRQAGYGQGAGASAGAAAAGAGAGRQAGYGQGAGASAGAAAAGADAGYGGQAGYGQGAGASAGAAASGAGAGYGGQAGYGQGAGASAGAAAAGAGAGYLGQAGYGQGAGASAGAAAGAGAGYGGQAGYGQGTGAAASAAASSAGAGAGYRGQAGYIQGAGASAGAAAAGAGVGYGGQAGYGQGAGASAGAAAAAGAGAGRQAGYGQGAGASAGAAAAGAGAGRQAGYGQGAGASAGAAAAGADAGYGGQAGYGQGAGASAGAAASGAGAGYGGQAGYGQGAGASAGAAAAGAGAGYLGQAGYGQGAGASAGAAAGAGAGYGGQAGYGQGTGAAASAAASSA

SEQ ID NO: 54 - protein, 792 aa, Artificial Sequence
  Description of Artificial Sequence: Synthetic polypeptide
GAGAGYRGQAGYIQGAGASAGAAAAGAGVGYGGQAGYGQGAGASAGAAAAAGAGAGRQAGYGQGAGASAGAAAAGAGAGRQAGYGQGAGASAGAAAAGADAGYGGQAGYGQGAGASAGAAASGAGAGYGGQAGYGQGAGASAGAAAAGAGAGYLGQAGYGQGAGASAGAAAGAGAGYGGQAGYGQGTGAAASAAASSAGAGAGYRGQAGYIQGAGASAGAAAAGAGVGYGGQAGYGQGAGASAGAAAAAGAGAGRQAGYGQGAGASAGAAAAGAGAGRQAGYGQGAGASAGAAAAGADAGYGGQAGYGQGAGASAGAAASGAGAGYGGQAGYGQGAGASAGAAAAGAGAGYLGQAGYGQGAGASAGAAAGAGAGYGGQAGYGQGTGAAASAAASSAGAGAGYRGQAGYIQGAGASAGAAAAGAGVGYGGQAGYGQGAGASAGAAAAAGAGAGRQAGYGQGAGASAGAAAAGAGAGRQAGYGQGAGASAGAAAAGADAGYGGQAGYGQGAGASAGAAASGAGAGYGGQAGYGQGAGASAGAAAAGAGAGYLGQAGYGQGAGASAGAAAGAGAGYGGQAGYGQGTGAAASAAASSAGAGAGYRGQAGYIQGAGASAGAAAAGAGVGYGGQAGYGQGAGASAGAAAAAGAGAGRQAGYGQGAGASAGAAAAGAGAGRQAGYGQGAGASAGAAAAGADAGYGGQAGYGQGAGASAGAAASGAGAGYGGQAGYGQGAGASAGAAAAGAGAGYLGQAGYGQGAGASAGAAAGAGAGYGGQAGYGQGTGAAASAAASSA

SEQ ID NO: 55 - protein, 1584 aa, Artificial Sequence
  Description of Artificial Sequence: Synthetic polypeptide
GAGAGYRGQAGYIQGAGASAGAAAAGAGVGYGGQAGYGQGAGASAGAAAAAGAGAGRQAGYGQGAGASAGAAAAGAGAGRQAGYGQGAGASAGAAAAGADAGYGGQAGYGQGAGASAGAAASGAGAGYGGQAGYGQGAGASAGAAAAGAGAGYLGQAGYGQGAGASAGAAAGAGAGYGGQAGYGQGTGAAASAAASSAGAGAGYRGQAGYIQGAGASAGAAAAGAGVGYGGQAGYGQGAGASAGAAAAAGAGAGRQAGYGQGAGASAGAAAAGAGAGRQAGYGQGAGASAGAAAAGADAGYGGQAGYGQGAGASAGAAASGAGAGYGGQAGYGQGAGASAGAAAAGAGAGYLGQAGYGQGAGASAGAAAGAGAGYGGQAGYGQGTGAAASAAASSAGAGAGYRGQAGYIQGAGASAGAAAAGAGVGYGGQAGYGQGAGASAGAAAAAGAGAGRQAGYGQGAGASAGAAAAGAGAGRQAGYGQGAGASAGAAAAGADAGYGGQAGYGQGAGASAGAAASGAGAGYGGQAGYGQGAGASAGAAAAGAGAGYLGQAGYGQGAGASAGAAAGAGAGYGGQAGYGQGTGAAASAAASSAGAGAGYRGQAGYIQGAGASAGAAAAGAGVGYGGQAGYGQGAGASAGAAAAAGAGAGRQAGYGQGAGASAGAAAAGAGAGRQAGYGQGAGASAGAAAAGADAGYGGQAGYGQGAGASAGAAASGAGAGYGGQAGYGQGAGASAGAAAAGAGAGYLGQAGYGQGAGASAGAAAGAGAGYGGQAGYGQGTGAAASAAASSAGAGAGYRGQAGYIQGAGASAGAAAAGAGVGYGGQAGYGQGAGASAGAAAAAGAGAGRQAGYGQGAGASAGAAAAGAGAGRQAGYGQGAGASAGAAAAGADAGYGGQAGYGQGAGASAGAAASGAGAGYGGQAGYGQGAGASAGAAAAGAGAGYLGQAGYGQGAGASAGAAAGAGAGYGGQAGYGQGTGAAASAAASSAGAGAGYRGQAGYIQGAGASAGAAAAGAGVGYGGQAGYGQGAGASAGAAAAAGAGAGRQAGYGQGAGASAGAAAAGAGAGRQAGYGQGAGASAGAAAAGADAGYGGQAGYGQGAGASAGAAASGAGAGYGGQAGYGQGAGASAGAAAAGAGAGYLGQAGYGQGAGASAGAAAGAGAGYGGQAGYGQGTGAAASAAASSAGAGAGYRGQAGYIQGAGASAGAAAAGAGVGYGGQAGYGQGAGASAGAAAAAGAGAGRQAGYGQGAGASAGAAAAGAGAGRQAGYGQGAGASAGAAAAGADAGYGGQAGYGQGAGASAGAAASGAGAGYGGQAGYGQGAGASAGAAAAGAGAGYLGQAGYGQGAGASAGAAAGAGAGYGGQAGYGQGTGAAASAAASSAGAGAGYRGQAGYIQGAGASAGAAAAGAGVGYGGQAGYGQGAGASAGAAAAAGAGAGRQAGYGQGAGASAGAAAAGAGAGRQAGYGQGAGASAGAAAAGADAGYGGQAGYGQGAGASAGAAASGAGAGYGGQAGYGQGAGASAGAAAAGAGAGYLGQAGYGQGAGASAGAAAGAGAGYGGQAGYGQGTGAAASAAASSA

SEQ ID NO: 56 - protein, 482 aa, Artificial Sequence
  Description of Artificial Sequence: Synthetic polypeptide
GAGAGGAGYGRGAGAGAGAAAGAGAGAAAGAGAGAGGYGGQGGYGAGAGAGAAAAAGAGAGGAAGYSRGGRAGAAGAGAGAAAGAGAGAGGYGGQGGYGAGAGAGAAAAAGAGSGGAGGYGRGAGAGAAAGAGAAAGAGAGAGGYGGQGGYGAGAGAAAAAGAGAGGAGYGRGAGAGAGAAAGAGAGAAAGAGAGAGGYGGQGGYGAGAGAGAAAAAGAGAGGAAGYSRGGRAGAAGAGAGAAAGAGAGAGGYGGQGGYGAGAGAGAAAAAGAGSGGAGGYGRGAGAGAAAGAGAAAGAGAGAGGYGGQGGYGAGAGAAAAAGAGAGGAGYGRGAGAGAGAAAGAGAGAAAGAGAGAGGYGGQGGYGAGAGAGAAAAAGAGAGGAAGYSRGGRAGAAGAGAGAAAGAGAGAGGYGGQGGYGAGAGAGAAAAAGAGSGGAGGYGRGAGAGAAAGAGAAAGAGAGAGGYGGQGGYGAGAGAAAA

SEQ ID NO: 57 - protein, 810 aa, Artificial Sequence
  Description of Artificial Sequence: Synthetic polypeptide
GGYGAVAGGSGAGASAGVGAGAGSVAGYGGQGGYGAGTGAGAGSAGGYGRGTGAGTAAGSGAGAAAGAGAGAAAGAGAGAAAGAGAGAGSLGGYEGQGAYSAGVGAGAAAAAGAGAGSVGGYGRGAGVGAGAAAGSAAGAGGAGGYRRDGGYGAGAGAGATAAASSGAGSAGGYGRGAGAGAAAVAGADAGGYGRNSGAGTAAYAGARAGSAGVYGGQGGYSSGAGASAASGAGADITSGYGRGDGAGAGAGTIGAGGYGGGAGSGAAAAGGYGAVAGGSGAGASAGVGAGAGSVAGYGGQGGYGAGTGAGAGSAGGYGRGTGAGTAAGSGAGAAAGAGAGAAAGAGAGAAAGAGAGAGSLGGYEGQGAYSAGVGAGAAAAAGAGAGSVGGYGRGAGVGAGAAAGSAAGAGGAGGYRRDGGYGAGAGAGATAAASSGAGSAGGYGRGAGAGAAAVAGADAGGYGRNSGAGTAAYAGARAGSAGVYGGQGGYSSGAGASAASGAGADITSGYGRGDGAGAGAGTIGAGGYGGGAGSGAAAAGGYGAVAGGSGAGASAGVGAGAGSVAGYGGQGGYGAGTGAGAGSAGGYGRGTGAGTAAGSGAGAAAGAGAGAAAGAGAGAAAGAGAGAGSLGGYEGQGAYSAGVGAGAAAAAGAGAGSVGGYGRGAGVGAGAAAGSAAGAGGAGGYRRDGGYGAGAGAGATAAASSGAGSAGGYGRGAGAGAAAVAGADAGGYGRNSGAGTAAYAGARAGSAGVYGGQGGYSSGAGASAASGAGADITSGYGRGDGAGAGAGTIGAGGYGGGAGSGAAAA

SEQ ID NO: 58 - protein, 790 aa, Artificial Sequence
  Description of Artificial Sequence: Synthetic polypeptide
GSGAGAGSGYGAGAGAGAGSGYGAGSSASAGSAINTQTVTSSTTTSSQSSAAATGAGYGTGAGTGASAGAAASGAGAGYGGQAGYGQGAGASARAAGSGYGAGAGAAAAAGSGYGAGAGAGAGSGYGAGAAAGSGAGAGSGYGAGAGAGAGSGYGAGSSASAGSAINTQTVTSSTTTSSQSSAAATGAGYGTGAGTGASAGAAASGAGAGYGGQAGYGQGAGASARAAGSGYGAGAGAAAAAGSGYGAGAGAGAGSGYGAGAAAGSGAGAGSGYGAGAGAGAGSGYGAGSSASAGSAINTQTVTSSTTTSSQSSAAATGAGYGTGAGTGASAGAAASGAGAGYGGQAGYGQGAGASARAAGSGYGAGAGAAAAAGSGYGAGAGAGAGSGYGAGAAGSGAGAGSGYGAGAGAGAGSGYGAGSSASAGSAINTQTVTSSTTTSSQSSAAATGAGYGTGAGTGASAGAAASGAGAGYGGQAGYGQGAGASARAAGSGYGAGAGAAAAAGSGYGAGAGAGAGSGYGAGAAAGSGAGAGSGYGAGAGAGAGSGYGAGSSASAGSAINTQTVTSSTTTSSQSSAAATGAGYGTGAGTGASAGAAASGAGAGYGGQAGYGQGAGASARAAGSGYGAGAGAAAAAGSGYGAGAGAGAGSGYGAGAAAGSGAGAGSGYGAGAGAGAGSGYGAGSSASAGSAINTQTVTSSTTTSSQSSAAATGAGYGTGAGTGASAGAAASGAGAGYGGQAGYGQGAGASARAAGSGYGAGAGAAAAAGSGYGAGAGAGAGSGYGAGAA

SEQ ID NO: 59 - protein, 395 aa, Artificial Sequence
  Description of Artificial Sequence: Synthetic polypeptide
GSGAGAGSGYGAGAGAGAGSGYGAGSSASAGSAINTQTVTSSTTTSSQSSAAATGAGYGTGAGTGASAGAAASGAGAGYGGQAGYGQGAGASARAAGSGYGAGAGAAAAAGSGYGAGAGAGAGSGYGAGAAAGSGAGAGSGYGAGAGAGAGSGYGAGSSASAGSAINTQTVTSSTTTSSQSSAAATGAGYGTGAGTGASAGAAASGAGAGYGGQAGYGQGAGASARAAGSGYGAGAGAAAAAGSGYGAGAGAGAGSGYGAGAAAGSGAGAGSGYGAGAGAGAGSGYGAGSSASAGSAINTQTVTSSTTTSSQSSAAATGAGYGTGAGTGASAGAAASGAGAGYGGQAGYGQGAGASARAAGSGYGAGAGAAAAAGSGYGAGAGAGAGSGYGAGAA

SEQ ID NO: 60 - protein, 12 aa, Artificial Sequence
  Description of Artificial Sequence: Synthetic peptide
GGGGGGHHHHHH

SEQ ID NO: 61 - protein, 25 aa, Artificial Sequence
  Description of Artificial Sequence: Synthetic peptide
GDYKDDDDKDYKDDDDKDYKDDDDK

SEQ ID NO: 62 - protein, 32 aa, Artificial Sequence
  Description of Artificial Sequence: Synthetic polypeptide
GDYKDDDDKDYKDDDDKDYKDDDDKGHHHHHH

SEQ ID NO: 63 - protein, 5 aa, Unknown
  Description of Unknown: silk protein sequence
AAAAA

SEQ ID NO: 64 - protein, 5 aa, Unknown
  Description of Unknown: silk protein sequence
SGAGG

SEQ ID NO: 65 - protein, 5 aa, Unknown
  Description of Unknown: silk protein sequence
GSGAG

SEQ ID NO: 66 - protein, 5 aa, Unknown
  Description of Unknown: silk protein sequence
GGSGA

SEQ ID NO: 67 - protein, 8 aa, Artificial Sequence
  Description of Artificial Sequence: Synthetic peptide
  MISC_FEATURE (1)..(8): This sequence may encompass 6-8 residues
HHHHHHHH

SEQ ID NO: 68 - protein, 6 aa, Artificial Sequence
  Description of Artificial Sequence: Synthetic 6xHis tag
HHHHHH

SEQ ID NO: 69 - protein, 11 aa, Artificial Sequence
  Description of Artificial Sequence: Synthetic peptide
GGGGGHHHHHH

(19) United States
(12) Patent Application Publication
(10) Pub. No.: US 2022/0289790 A1
(43) Pub. Date: Sep. 15, 2022

(54) METHODS FOR IMPROVED EXTRACTION OF SPIDER SILK PROTEINS

(71) Applicant: Bolt Threads, Inc., Emeryville, CA (US)

(72) Inventors: Phillip Mui, Fremont, CA (US); Simon Li, San Francisco, CA (US); Ritu Bansal Mutalik, Albany, CA (US); Cole Rich Peterson, Emeryville, CA (US)

(21) Appl. No.: 17/633,498

(22) PCT Filed: Aug. 21, 2020
(86) PCT No.: PCT/US2020/047514

§ 371 Date: Feb. 7, 2022

Related U.S. Application Data
(60) Provisional application No. 62/890,473, filed on Aug. 22, 2019.

(51) Int. Cl. C07K 1/34; C07K 14/435; C12N 15/70

(52) U.S. Cl. CPC C07K 1/34; C07K 14/43518; C12N 15/70; C07K 2319/02; C07K 2319/21; C12P 21/02

(57) ABSTRACT

Provided herein are methods of improving solubilization, extraction, and isolation of recombinant spider silk proteins with a salt and alcohol buffer. Provided herein are methods of solubilizing a recombinant spider silk protein from a host cell, comprising: providing a cell culture comprising a host cell, wherein the host cell expresses a recombinant spider silk protein; collecting an insoluble portion derived from the cell culture, wherein the insoluble portion comprises the recombinant spider silk protein; adding the insoluble portion of the host cell to a solution comprising a salt and an alcohol, thereby solubilizing the recombinant spider silk protein in the solution.

### CROSS REFERENCE TO RELATED APPLICATIONS

This application claims the benefit of U.S. Provisional Application No. 62/890,473, filed Aug. 22, 2019, which is hereby incorporated in its entirety by reference.

### SEQUENCE LISTING

The instant application contains a Sequence Listing which has been submitted via EFS-Web and is hereby incorporated by reference in its entirety. Said ASCII copy, created on Month XX, 20XX, is named XXXXXUS_sequencelisting.txt, and is X,XXX,XXX bytes in size.

### BACKGROUND

Spider's silk polypeptides are large (>150 kDa, >1000 amino acids) polypeptides that can be broken down into three domains: an N-terminal non-repetitive domain (NTD), the repeat domain (REP), and the C-terminal non-repetitive domain (CTD). The NTD and CTD are relatively small (˜150, ˜100 amino acids respectively), well-studied, and are believed to confer to the polypeptide aqueous stability, pH sensitivity, and molecular alignment upon aggregation. NTD also has a strongly predicted secretion tag, which is often removed during heterologous expression. The repetitive region composes ˜90% of the natural polypeptide, and folds into the crystalline and amorphous regions that confer strength and flexibility to the silk fiber, respectively.

Recombinant spider silk polypeptides form undesirable insoluble aggregates during production and purification. Due to their ability to aggregate and form β-sheet structures, proteins based on silk sequences are difficult to solubilize. Solubilization of these proteins often requires harsh chemical conditions for biological molecules which often degrades the proteins, resulting in poor yield and solids or fibers with low tenacity and poor hand feel. Improved methods to purify these polypeptides that result in increased solubility and recovery of the silk proteins are therefore required.

### SUMMARY OF THE INVENTION

Provided herein are methods of solubilizing a recombinant spider silk protein from a host cell, comprising: providing a cell culture comprising a host cell, wherein the host cell expresses a recombinant spider silk protein; collecting an insoluble portion derived from the cell culture, wherein the insoluble portion comprises the recombinant spider silk protein; adding the insoluble portion of the host cell to a solution comprising a salt and an alcohol, thereby solubilizing the recombinant spider silk protein in the solution.

In some embodiments, the salt comprises a calcium salt. In some embodiments, the calcium salt comprises at least one of calcium chloride, calcium nitrate, calcium thiocyanate, calcium iodide, or calcium bromide. In some embodiments, the calcium salt comprises calcium chloride.

In some embodiments, the solution comprises 1M, 1.5M, 2M, 2.5M, 3M, or 4M calcium chloride. In some embodiments, the solution comprises 2M calcium chloride. In some embodiments, the calcium salt comprises calcium nitrate.

In some embodiments, the salt comprises a strontium salt or a barium salt.

In some embodiments, the insoluble portion is at least 5%, 10%, 15%, 20%, 25%, 30%, or 35% (w/v) of the solution volume. In some embodiments, the insoluble portion is about 15% (w/v) of the solution volume. In some embodiments, the insoluble portion is at most about 35% (w/v) of the solution volume.

In some embodiments, the ratio of the volume of the solution to the insoluble portion is at least 3×, 5× or 7×. In some embodiments, the ratio of the volume of the solution to the insoluble portion is at least 3×. In some embodiments, the ratio of the volume of the solution to the insoluble portion is about 7×.

In some embodiments, the alcohol comprises at least one of methanol, ethanol, or isopropanol. In some embodiments, the alcohol comprises methanol. In some embodiments, the solution comprises 2M calcium chloride and methanol.

In some embodiments, the insoluble portion is incubated with the solution at a temperature between 20° C. and 70° C. In some embodiments, the insoluble portion is incubated at room temperature. In some embodiments, the insoluble portion is incubated at about 35° C. In some embodiments, the insoluble portion is incubated at about 55° C. In some embodiments, the insoluble portion is incubated at no more than 70° C. In some embodiments, the insoluble portion is incubated at no less than 20° C.

In some embodiments, the insoluble portion is incubated in the solution for 15 to 120 minutes. In some embodiments, the insoluble portion is incubated in the solution for 30 min. In some embodiments, the method further comprises evaporating the alcohol.

In some embodiments, the insoluble portion comprises a cell lysate pellet. In some embodiments, collecting the insoluble portion derived from the cell culture comprises lysing the host cell. In some embodiments, lysing comprises heat treatment, chemical treatment, shear disruption, physical homogenization, microfluidization, sonication, or chemical homogenization.

In some embodiments, collecting the insoluble portion of the cell culture further comprises centrifuging the lysed cell to obtain a cell lysate pellet.

In some embodiments, the method further comprises removing impurities from the solution. In some embodiments, removing impurities comprises adding an aqueous solution to precipitate the impurities. In some embodiments, the aqueous solution comprises water.

In some embodiments, removing the impurities comprises filtration, centrifugation, gravitational settling, adsorption, dialysis, or phase separation. In some embodiments, the filtration is ultrafiltration, microfiltration, or diafiltration.

In some embodiments, the method further comprises isolating the recombinant spider silk protein from the solution, thereby producing an isolated recombinant spider silk protein.

In some embodiments, an amount of isolated recombinant spider silk protein is measured using a Western blot. In some embodiments, an amount of isolated recombinant spider silk protein is measured using an ELISA. In some embodiments, an amount of isolated recombinant spider silk protein is measured using Size Exclusion Chromatography.

In some embodiments, the isolated recombinant spider silk protein is a full-length recombinant spider silk protein.

In some embodiments, the isolated recombinant spider silk protein comprises at least 60%, at least 65%, at least 70%, at least 75%, at least 80%, at least 85%, at least 90%, or at least 95% full-length recombinant spider silk protein.

In some embodiments, an amount of full-length recombinant spider silk protein is measured using a Western blot. In some embodiments, an amount of full-length recombinant spider silk protein is measured using Size Exclusion Chromatography.

In some embodiments, the purity of the isolated recombinant spider silk protein is 5-10%, 10-15%, 15-20%, 20-25%, 25-30%, 30-35%, 35-40%, 45-50%, 50-55%, 55-60%, 60-65%, 65-70%, 70-75%, 75-80%, 80-85%, 85-90%, 09-95%, or 95-100%.

In some embodiments, the recombinant spider silk protein is a highly crystalline silk protein, a high beta sheet content silk protein, or a low solubility silk protein. In some embodiments, the cell culture comprises a fungal, a bacterial or a yeast cell. In some embodiments, the bacterial cell is Escherichia coli. In some embodiments, the method further comprises drying the isolated recombinant spider silk protein to produce a silk protein powder.

In another aspect, provided herein is a method of isolating a recombinant spider silk protein from a host cell, comprising: providing a cell culture comprising a host cell, wherein the host cell expresses a recombinant spider silk protein; collecting an insoluble portion derived from the cell culture, wherein the insoluble portion comprises the recombinant spider silk protein; adding the insoluble portion of the host cell to a solution comprising 2M calcium chloride and methanol, thereby solubilizing the recombinant spider silk protein in the solution; and isolating the recombinant spider silk protein from the solution, thereby producing an isolated recombinant spider silk protein. In some embodiments, the method further comprises drying the isolated recombinant spider silk protein to produce a silk protein powder.

In another aspect, provided herein are compositions comprising a recombinant spider silk protein produced by the method described herein.

In some embodiments, the composition comprises a recombinant spider silk protein powder. In some embodiments, the recombinant spider silk comprises at least 55%, at least 60%, at least 65%, at least 70%, at least 75%, at least 80%, at least 85%, at least 90%, at least 95%, or 100% full length recombinant spider silk.

In another aspect, provided herein are silk solids comprising a recombinant spider silk protein produced by the method described herein.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 shows an exemplary flow chart of the solubilization process.

FIG. 2 shows a second exemplary flow chart of the solubilization process.

FIG. 3 provides an immunoblot showing P0 spider silk protein extracted with calcium salts in methanol.

FIG. 4 provides a graph of the P0 spider silk protein in solution after incubation at 35° C. and 55° C. with agitation.

FIG. 5A shows SEC peak profiles of P0 spider silk protein after P0 protein fragments were removed after water precipitation. FIG. 5B shows the SEC peak profile after dialysis and lyophilization.

### DETAILED DESCRIPTION

### Definitions

Terms used in the claims and specification are defined as set forth below unless otherwise specified.

Unless otherwise defined herein, scientific and technical terms used in connection with the present methods and compositions described herein shall have the meanings that are commonly understood by those of ordinary skill in the art. Further, unless otherwise required by context, singular terms shall include the plural and plural terms shall include the singular. Generally, nomenclatures used in connection with, and techniques of, biochemistry, enzymology, molecular and cellular biology, microbiology, genetics and polypeptide and nucleic acid chemistry and hybridization described herein are those well-known and commonly used in the art.

The methods and techniques described herein are generally performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol I, CRC Press (1976); Handbook of Biochemistry: Section A Proteins, Vol II, CRC Press (1976); Essentials of Glycobiology, Cold Spring Harbor Laboratory Press (1999).

All publications, patents and other references mentioned herein are hereby incorporated by reference in their entireties.

The following terms, unless otherwise indicated, shall be understood to have the following meanings:

The term “in vitro” refers to processes that occur in a living cell growing separate from a living organism, e.g., growing in tissue culture.

The term “in vivo” refers to processes that occur in a living organism.

The term “clarifying” as use herein refers to a method removing host cell biomass, such as whole cells, lysed cells, membranes, lipids, organelles, nuclei, non-spider silk proteins, or any other undesirable cell part or product, or any other undesirable portion of a cell culture. Clarifying may also refer to removing impurities from a partially purified or isolated spider silk composition. Impurities may include, but are not limited to, non-spider silk proteins, degraded spider silk proteins, large aggregates of proteins, chemicals used during the purification and isolation process, or any other undesirable material.

The term “purity” as used herein refers to the amount of full-length isolated recombinant spider silk protein as a portion of all isolated components, such as partial or degraded isolated recombinant spider silk proteins, lipids, proteins, membranes, or other molecules in a sample, such as an extracted sample.

The term “silk solid” or “recombinant silk solid” refers to isolated recombinant spider silk compositions, such as fibers, extrudates, powders, or pellets. An extrudate is an extruded recombinant spider silk composition that has been extruded through a spinneret.

The term “polynucleotide” or “nucleic acid molecule” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation.

Unless otherwise indicated, and as an example for all sequences described herein under the general format “SEQ ID NO:”, “nucleic acid comprising SEQ ID NO:1” refers to a nucleic acid, at least a portion of which has either (i) the sequence of SEQ ID NO:1, or (ii) a sequence complementary to SEQ ID NO:1. The choice between the two is dictated by the context. For instance, if the nucleic acid is used as a probe, the choice between the two is dictated by the requirement that the probe be complementary to the desired target.

An “isolated” RNA, DNA or a mixed polymer is one which is substantially separated from other cellular components that naturally accompany the native polynucleotide in its natural host cell, e.g., ribosomes, polymerases and genomic sequences with which it is naturally associated.

The term “recombinant” refers to a biomolecule, e.g., a gene or polypeptide, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as polypeptides and/or mRNAs encoded by such nucleic acids.

As used herein, an endogenous nucleic acid sequence in the genome of an organism (or the encoded polypeptide product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “recombinant” because it is separated from at least some of the sequences that naturally flank it. In an embodiment, a heterologous nucleic acid molecule is not endogenous to the organism. In further embodiments, a heterologous nucleic acid molecule is a plasmid or molecule integrated into a host chromosome by homologous or random integration.

A nucleic acid is also considered “recombinant” if it contains any modifications that do not naturally occur to the corresponding nucleic acid in a genome. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome.

The term “percent sequence identity” in the context of nucleic acid sequences refers to the quantitative value of an alignment of the residues in the two sequences when aligned for maximum correspondence. The length of sequence identity comparison may be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA provides alignments and percent sequence identity of the regions of the best overlap between the query and search sequences. Pearson, Methods Enzymol. 183:63-98 (1990) (hereby incorporated by reference in its entirety). For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference. Alternatively, sequences can be compared using the computer program, BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

The term “substantial homology” or “substantial similarity,” when referring to a nucleic acid or fragment thereof, indicates that, when optimally aligned with appropriate nucleotide insertions or deletions with another nucleic acid (or its complementary strand), there is nucleotide sequence identity in at least about 76%, 80%, 85%, preferably at least about 90%, and more preferably at least about 95%, 96%, 97%, 98% or 99% of the nucleotide bases, as measured by any well-known algorithm of sequence identity, such as FASTA, BLAST or Gap, as discussed above.

Nucleic acids (also referred to as polynucleotides) can include both sense and antisense strands of RNA, cDNA, genomic DNA, and synthetic forms and mixed polymers of the above. They can be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.) Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule. Other modifications can include, for example, analogs in which the ribose ring contains a bridging moiety or other structure such as the modifications found in “locked” nucleic acids.

The term “mutated” when applied to nucleic acid sequences means that nucleotides in a nucleic acid sequence may be inserted, deleted or changed compared to a reference nucleic acid sequence. A single alteration may be made at a locus (a point mutation) or multiple nucleotides may be inserted, deleted or changed at a single locus. In addition, one or more alterations may be made at any number of loci within a nucleic acid sequence. A nucleic acid sequence may be mutated by any method known in the art including but not limited to mutagenesis techniques such as “error-prone PCR” (a process for performing PCR under conditions where the copying fidelity of the DNA polymerase is low, such that a high rate of point mutations is obtained along the entire length of the PCR product; see, e.g., Leung et al., Technique, 1:11-15 (1989) and Caldwell and Joyce, PCR Methods Applic. 2:28-33 (1992)); and “oligonucleotide-directed mutagenesis” (a process which enables the generation of site-specific mutations in any cloned DNA segment of interest; see, e.g., Reidhaar-Olson and Sauer, Science 241:53-57 (1988)).

The term “vector” as used herein is intended to refer to a nucleic acid molecule capable of transporting another nucleic acid to which it has been linked. One type of vector is a “plasmid,” which generally refers to a circular double stranded DNA loop into which additional DNA segments may be ligated, but also includes linear double-stranded molecules such as those resulting from amplification by the polymerase chain reaction (PCR) or from treatment of a circular plasmid with a restriction enzyme. Other vectors include cosmids, bacterial artificial chromosomes (BAC) and yeast artificial chromosomes (YAC). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain preferred vectors are capable of directing the expression of genes to which they are operatively linked. Such vectors are referred to herein as “recombinant expression vectors” (or simply “expression vectors”).

The term “expression system” as used herein includes vehicles or vectors for the expression of a gene in a host cell as well as vehicles or vectors which bring about stable integration of a gene into the host chromosome.

“Operatively linked” or “operably linked” expression control sequences refers to a linkage in which the expression control sequence is contiguous with the gene of interest to control the gene of interest, as well as expression control sequences that act in trans or at a distance to control the gene of interest.

The term “expression control sequence” as used herein refers to polynucleotide sequences which are necessary to affect the expression of coding sequences to which they are operatively linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance polypeptide stability; and when desired, sequences that enhance polypeptide secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “promoter,” as used herein, refers to a DNA region to which RNA polymerase binds to initiate gene transcription, and positions at the 5′ direction of an mRNA transcription initiation site.

The term “recombinant host cell” (or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism.

The term “polypeptide” encompasses both naturally-occurring and non-naturally-occurring proteins, and fragments, mutants, derivatives and analogs thereof. A polypeptide may be monomeric or polymeric. Further, a polypeptide may comprise a number of different domains each of which has one or more distinct activities.

As used herein, the term “molecule” means any compound, including, but not limited to, a small molecule, peptide, polypeptide, sugar, nucleotide, nucleic acid, polynucleotide, lipid, etc., and such a compound can be natural or synthetic.

The term “block” or “repeat unit” as used herein refers to a subsequence greater than approximately 12 amino acids of a natural silk polypeptide that is found, possibly with modest variations, repeatedly in the natural silk polypeptide sequence and serves as a basic repeating unit in the silk polypeptide sequence. Blocks may, but do not necessarily, include very short “motifs.” A “motif” as used herein refers to an approximately 2-10 amino acid sequence that appears in multiple blocks. For example, a motif may consist of the amino acid sequence GGA, GPG, or AAAAA. A sequence of a plurality of blocks is a “block copolymer.”

As used herein, the term “repeat domain” refers to a sequence selected from the set of contiguous (unbroken by a substantial non-repetitive domain, excluding known silk spacer elements) repetitive segments in a silk polypeptide. Native silk sequences generally contain one repeat domain. In some embodiments, there is one repeat domain per silk molecule. A “macro-repeat” as used herein is a naturally occurring repetitive amino acid sequence comprising more than one block. In an embodiment, a macro-repeat is repeated at least twice in a repeat domain. In a further embodiment, the two repetitions are imperfect. A “quasi-repeat” as used herein is an amino acid sequence comprising more than one block, such that the blocks are similar but not identical in amino acid sequence.

A “repeat sequence” or “R” as used herein refers to a repetitive amino acid sequence. In an embodiment, a repeat sequence includes a macro-repeat or a fragment of a macro-repeat. In another embodiment, a repeat sequence includes a block. In a further embodiment, a single block is split across two repeat sequences.

The term “about” indicates and encompasses an indicated value and a range above and below that value. In certain embodiments, the term “about” indicates the designated value ±10%, ±5%, or ±1%. In certain embodiments, where applicable, the term “about” indicates the designated value(s) ±one standard deviation of that value(s).

It must be noted that, as used in the specification and the appended claims, the singular forms “a,” “an” and “the” include plural referents unless the context clearly dictates otherwise.

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50. In addition, a range of 2-5% includes 2% and 5%, and any number or fraction of a number in between, for example: 2.25%, 2.5%, 2.75%, 3%, 3.25%, 3.5%, 3.75%, 4%, 4.25%, 4.5%, and 4.75%.

Methods for Solubilizing a Recombinant Protein

Recombinant spider silk protein expressed in cell culture must be purified away from the cell components. In some instances, the silk protein is trapped in insoluble cell debris, or forms insoluble silk protein aggregates. Insoluble silk protein is difficult to purify and results in decreased recombinant silk protein recovery. In such cases, various methods can be applied to the insoluble cell debris or aggregate that releases the silk protein and solubilizes it for purification, which results in increased recovery of the recombinant silk protein.

Solubilization Process

Described herein are methods for solubilizing recombinant spider silk proteins, resulting in improved extraction and purification of such proteins from host cells. In some instances, the recombinant spider silk proteins are crystalline silk proteins. Crystalline silk proteins have lower solubility in solution than non-crystalline silk proteins.

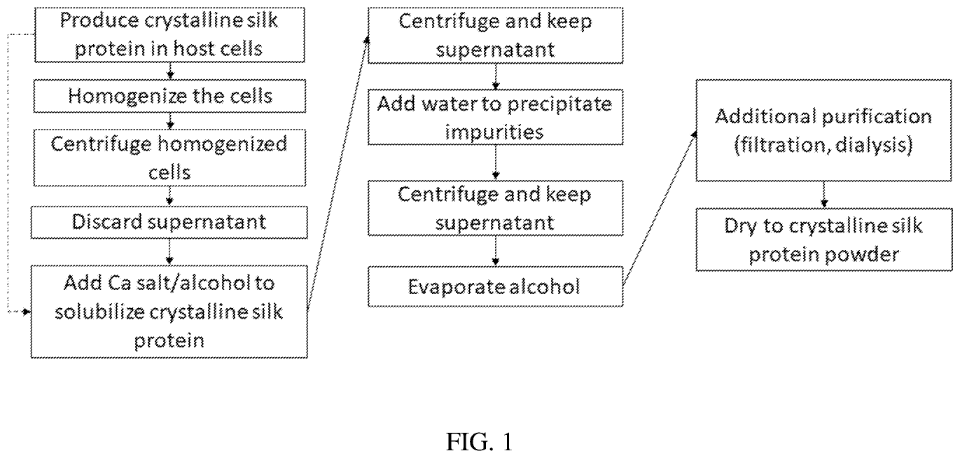

An exemplary solubilization and purification process is shown in FIG. 1. Optional flow steps are shown with dashed lines. First, the silk protein is expressed in transformed host cells. The host cells are then homogenized, the insoluble cell material including the silk protein is pelleted via centrifugation, the supernatant is discarded, and the insoluble material is resuspended in a solution comprising a salt and an alcohol. In one example, the salt is calcium chloride, and the alcohol is methanol. Alternatively, the host cells can be added directly to the salt/alcohol solution, which lyses the cells and releases the silk protein. The silk protein is incubated in the salt/alcohol solution, resulting in increased solubilization of the protein, and the remaining insoluble matter is pelleted again via centrifugation. At this point, the supernatant with the soluble silk protein is retained and undergoes further steps to remove non-silk protein impurities. In some instances, the addition of water is used to precipitate the non-silk protein impurities. The precipitated impurities can be removed again via centrifugation and discarded. The alcohol supernatant with the soluble silk protein is retained and the alcohol is evaporated. Addition purifications can be performed on the extracted silk protein, such as filtration or dialysis, which is then dried to produce a powder. This solubilization process requires an explosion-proof centrifuge, as the supernatants with the solubilized silk protein contains alcohol.

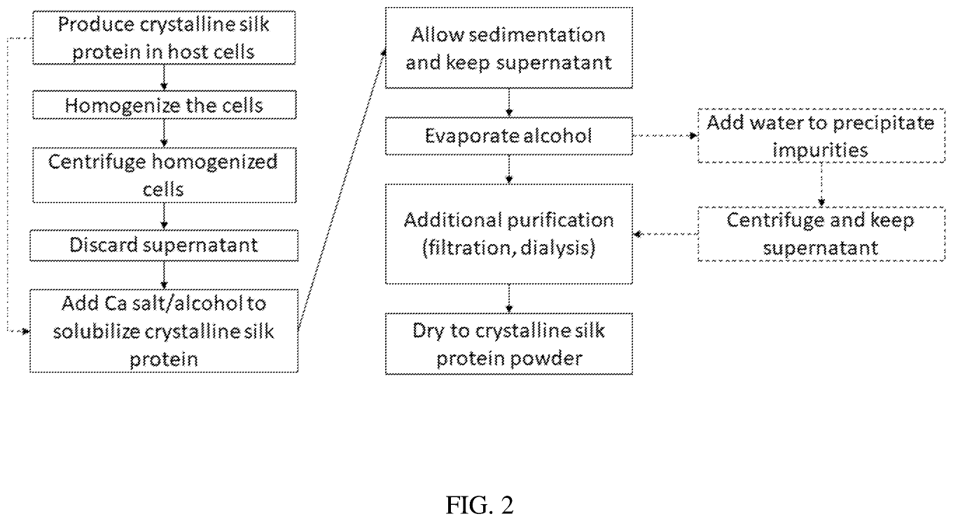

An second exemplary solubilization and purification process is shown in FIG. 2. Optional flow steps are shown with dashed lines. In this example, the initial production and lysing of the host cells is the same as in the previous exemplary solubilization process. The silk protein is expressed in host cells which are lysed, the insoluble portion with the silk protein is pelleted and then resuspended in a solution comprising a salt and an alcohol. At this point, the non-solubilized cell matter is allowed to sediment via gravity, not centrifugation. The alcohol supernatant with the soluble silk protein is collected, and the alcohol is evaporated. The supernatant with the soluble silk protein undergoes further steps to remove non-silk protein impurities. In some instances, the addition of water is used to precipitate the non-silk protein impurities. The precipitated impurities can be removed again via centrifugation and discarded. Addition purifications can be performed on the extracted silk protein, such as filtration or dialysis, which is then dried to produce a powder. This solubilization process does not require an explosion-proof centrifuge.

In some embodiments, “soluble” or “solubilized” refer to the portion of spider silk protein that is dissolved in a solution. In some embodiments, “solubilization” refers to the process in which a portion of a spider silk protein is dissolved in a solution.

In some embodiments, the portion of solubilized spider silk protein is from about 1-100% w/w, 1-10% w/w, 1-5% w/w, 5-10% w/w, 10-15% w/w, 15-20% w/w, 20-25% w/w, 25-30% w/w, 30-35% w/w, 35-40% w/w, 40-45% w/w, 45-50% w/w, 50-55% w/w, 55-60% w/w, 60-65% w/w, 65-70% w/w, 70-75% w/w, 75-80% w/w, 80-85% w/w, 85-90% w/w, 90-95% w/w, or 95-100% w/w of the total spider silk. In some embodiments, the portion of solubilized spider silk protein is at least about 1% w/w, 5% w/w, 10% w/w, 15% w/w, 20, 25% w/w, 30% w/w, 35% w/w, 40% w/w, 45% w/w, 50% w/w, 55% w/w, 60% w/w, 65% w/w, 70% w/w, 75% w/w, 80% w/w, 85% w/w, 90% w/w, 95% w/w, 99% w/w, or 100% w/w of the total spider silk. In some embodiments, insoluble refers to the portion of spider silk protein that is not dissolved in a solution. In some embodiments, the portion of insoluble spider silk protein is from about 1-100% w/w, 1-10% w/w, 1-5% w/w, 5-10% w/w, 10-15% w/w, 15-20% w/w, 20-25% w/w, 25-30% w/w, 30-35% w/w, 35-40% w/w, 40-45% w/w, 45-50% w/w, 50-55% w/w, 55-60% w/w, 60-65% w/w, 65-70% w/w, 70-75% w/w, 75-80% w/w, 80-85% w/w, 85-90% w/w, 90-95% w/w, or 95-100% w/w of the total spider silk.

Salts

In some embodiments, salt is added to the insoluble cell portion, pellet, or lysate to solubilize the recombinant spider silk protein. Appropriate salts include but are not limited to, salts with calcium ions, strontium ions, barium ions, magnesium ions, lithium ions, sodium ions, potassium ions, or ammonium ions. Such salts include, but are not limited to, calcium chloride, calcium nitrate, calcium thiocyanate, calcium carbonate, calcium fluoride, calcium iodide, calcium oxalate, calcium phosphate, calcium sulfate, calcium bromide, strontium bromide, strontium carbonate, strontium chloride, strontium fluoride, strontium iodide, strontium nitrate, barium chloride, barium bromide, barium iodide, barium acetate, barium cyanide, barium nitrate, barium sulfate, barium carbonate, barium sulfide, barium fluoride, barium manganate, barium phosphate, barium carbonate, sodium nitrate, sodium chloride, sodium bromide, sodium iodide, sodium fluoride, potassium nitrate, potassium chloride, potassium bromide, potassium fluoride, potassium iodide, or any combination thereof. In some embodiments, the salt is calcium chloride, calcium bromide, calcium iodide, strontium chloride, strontium bromide, strontium iodide, barium chloride, barium bromide, barium iodide, or any combination thereof. In some embodiments, the salt is a calcium salt. In some embodiments, the salt is calcium chloride. In some embodiments, the salt is calcium iodide. In some embodiments, the salt is calcium bromide. In some embodiments, the salt is calcium nitrate. In some embodiments, the salt is calcium thiocyanate. In some embodiments, the salt is a strontium salt. In some embodiments, the salt is strontium chloride, strontium iodide, or strontium bromide. In some embodiments, the salt is a barium salt. In some embodiments, the salt is barium chloride, barium iodide, or barium bromide.

Alcohols

In some embodiments, the insoluble cell portion, pellet, or lysate can be added to a solution comprising an alcohol to solubilize the recombinant spider silk protein. Any appropriate alcohol known in the art can be used, including but not limited to methanol, ethanol, isopropanol, isopropyl alcohol, n-propyl alcohol, butanol, pentanol, or any derivative thereof, or any combination thereof. Primary, secondary, or tertiary alcohols may be used. Exemplary primary alcohols include ethanol and methanol. Exemplary secondary alcohols include isopropyl alcohol and n-propyl alcohol. Exemplary tertiary alcohols include tert-butanol. In some embodiments, the alcohol is methanol. In some embodiments, the alcohol is ethanol. In some embodiments, the alcohol is isopropanol.

Buffer Conditions

The amount of the insoluble cell portion resuspend in the salt and acid solution can also be described as a volume to mass ratio. An exemplary volume to mass ratio is 3×, e.g., 300 ml of solution and 100 g of cell mass. In some embodiments, the insoluble cell portion mass to salt and alcohol solution volume ratio can be from between 1-10× mass to volume, 1-2× mass to volume, 1-3× mass to volume, 3-5× mass to volume, 5-7× mass to volume, 6-8× mass to volume, or 8-10× mass to volume. In some embodiments, the cell mass to salt and alcohol solution volume ratio can be at least 1×, 2×, 3×, 4×, 5×, 6×, 7×, 8×, 9×, or 10×. In some embodiments, the cell mass to salt and alcohol solution volume ratio is at least 3×. In some embodiments, the cell mass to salt and alcohol solution volume ratio is at most 3×. In some embodiments, the cell mass to salt and alcohol solution volume ratio is at least 5×. In some embodiments, the cell mass to salt and alcohol solution volume ratio is at least 7×. In some embodiments, the cell mass to salt and alcohol solution volume ratio is at least 9×.

The insoluble portion of the cell mass is resuspended in the salt and alcohol solution. The amount of cell mass in the final resuspension can be described as a percentage of cell mass to solution volume (weight by volume percentage). An exemplary weight by volume percentage of cell mass to solution volume is 100%, e.g., 100 mg cell mass and 100 ml solution. In some embodiments, the insoluble portion cell mass and salt and alcohol solution weight by volume can be from between 1-100%, 1-5%, 5-10%, 10-15%, 15-20%, 20-25%, 25-30%, 30-35%, 35-40%, 45-50%, 50-55%, 55-60%, 60-65%, 65-70%, 70-75%, 75-80%, 80-85%, 85-90%, 90-95%, or 95-100% w/v. In some embodiments, the insoluble portion cell mass and salt and alcohol solution weight by volume is at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% w/v.

In some embodiments, the insoluble portion cell mass and salt and alcohol solution weight by volume is about 15% (w/v). In some embodiments, the insoluble portion cell mass and salt and alcohol solution weight by volume is at most 35% (w/v).

In some embodiments, the concentration of the salt in the solution comprising the salt and alcohol solution and the insoluble cell portion, pellet, or lysate can be from between 0.01-10 M, 0.01-0.1 M, 0.1-0.5 M, 0.5-1 M, 1-2 M, 2-3 M, 3-4 M, 4-5 M, 5-6 M, 6-7 M, 7-8 M, 8-9 M, or 9-10 M. In some embodiments, the concentration of the salt in the solution comprising the salt and alcohol solution and the cell lysate or pellet can be at least about 0.1 M, 0.15 M, 0.2 M, 0.25 M, 0.3 M, 0.35 M, 0.4 M, 0.45 M, 0.5 M, 0.55 M, 0.6 M, 0.65 M, 0.7 M, 0.75 M, 0.8 M, 0.85 M, 0.9 M, 0.95 M, 1 M, 1.5 M, 2 M, 2.5 M, 3 M, 3.5 M, 4 M, 4.5 M, 5 M, 5.5 M, 6 M, 6.5 M, 7 M, 7.5 M, 8 M, 8.5 M, 9 M, 9.5 M, or 10 M. In some embodiments, the concentration of the salt in the solution is 1M, 1.5M, 2M, 2.5M or 3M. In some embodiments, the concentration of the salt in the solution is 2M.

Additional buffer modifications may also be used, such as shear protectants, viscosity modifiers, and/or solutes that affect vesicle structural properties. Excipients may also be added to improve the efficiency of the homogenization or microfluidization such as membrane softening materials and molecular crowding agents. Other modifications to the buffer may include specific pH ranges and/or concentrations of salts, organic solvents, small molecules, detergents, zwitterions, amino acids, polymers, and/or any combination of the above including multiple concentrations.

Incubation Time and Temperature

In some embodiments, the insoluble cell portion, pellet, or lysate is incubated with the solution comprising a salt and an alcohol for a determined amount of time. The amount of time the cell pellet or lysate is incubated with the solution can be altered to increase the solubilization of the spider silk protein or decrease any possible degradation of the protein. The incubation time can be from between 1 min to over 3 hours (180 min), 1 min to 60 min, 3 min to 90 min, 60 min to 120 min, 90 min to 150 min, or 120 min to 180 min. The incubation time can be at least 1 min, 5 min, 10 min, 15 min, 20 min, 30 min, 45 min, 60 min, 75 min, 90 min, 105 min, 120 min, 135 min, 150 min, 165 min, 180 min, or more. In some embodiments, the incubation time is 15 min. In some embodiments, the incubation time is 30 min. In some embodiments, the incubation time is 60 min. In some embodiments, the incubation time is 75 min. In some embodiments, the incubation time is 90 min. In some embodiments, the incubation time is 105 min. In some embodiments, the incubation time is 120 min.

The insoluble cell portion, pellet, or lysate can be incubated with the solution at 10-70° C. In some embodiments, the insoluble cell portion, pellet, or lysate is incubated with the solution at 10-20° C., 20-30° C., 20-22° C., 20-25° C., 25-20° C., 30-40° C., 30-35° C., 35-40° C., 40-55° C., 50-55° C., 55-60° C., or 60-70° C. In some embodiments, the insoluble cell portion, pellet, or lysate is incubated with the solution at 20-30° C. In some embodiments, the insoluble cell portion, pellet, or lysate is incubated with the solution at 22° C. In some embodiments, the insoluble cell portion, pellet, or lysate is incubated with the solution at 35° C. In some embodiments, the insoluble cell portion, pellet, or lysate is incubated with the solution at 55° C. In some embodiments, the insoluble cell portion, pellet, or lysate is incubated with the solution at no more than 70° C. In some embodiments, the insoluble cell portion, pellet, or lysate is incubated with the solution at no less than 20° C. In some embodiments, the insoluble cell portion, pellet, or lysate is incubated with the solution at room temperature.

In some embodiments, the recombinant spider silk protein is expressed in the cytoplasm of a host cell. Isolation of the protein requires lysing the host cell to release the recombinant spider silk protein. Any appropriate method can be used to lyse the host cell, including, but not limited to, heat treatment, chemical treatment, shear disruption, physical homogenization, sonication, or chemical homogenization. Chemical treatment includes incubating the cells with chemicals or enzymes known to disrupt the plasma membrane of prokaryotic and eukaryotic cells, such as detergents, such as Triton X-100, Nonidet P-40, CHAPS, sodium dodecyl sulfate (SDS), or other appropriate detergents.

The insoluble portion comprising the recombinant spider silk protein can be collected by centrifuging the cell lysate, resulting in a cell lysate pellet of insoluble material, including the recombinant spider silk protein. The centrifugation force or speed that pellets the insoluble recombinant protein can be determined by one of skill in the art. In some embodiments, the centrifuge speed is 100-10,000×g. In some embodiments, the centrifuge force is 100×g, 200×g, 300×g, 400×g, 500×g, 600×g, 700×g, 800×g, 900×g, 1000×g, 2000×g, 3000×g, 4000×g, 5000×g, 6000×g, 7000×g, 8000×g, 9000×g, or 10,000×g. Alternatively, the insoluble portion comprising the recombinant spider silk protein can be collected by sedimentation.

Impurities Removal

In some embodiments, biological or chemical impurities of non-spider silk protein can be removed from the solution comprising the solubilized spider silk protein. Removing impurities from the solution can be accomplished by filtration, absorption (e.g. charcoal or solid-state absorption), dialysis and phase separation induced by coacervation or the use of various chemicals. In other embodiments, phase separation may be chemically induced by adding a cosmotrope and/or a compound used to precipitate the protein from solution.

In some embodiments, impurities are removed using filtration, microfiltration, diafiltration and/or ultrafiltration (e.g., against deionized water). Membranes suitable for microfiltration may include 0.1 uM to 1 uM. Non-limiting examples of suitable membranes for ultrafiltration include hydrophobic membranes (e.g., PES, PS, cellulose acetate) with molecular weight cut-offs of between 50 kDa and 800 kDa, 100 kDa and 800 kDa, 200 kDa and 800 kDa, 300 kDa and 800 kDa, 400 kDa and 800 kDa, 500 kDa and 800 kDa, 600 kDa and 800 kDa, 700 kDa and 800 kDa, 100 kDa and 700 kDa, 200 kDa and 700 kDa, 300 kDa and 700 kDa, 400 kDa and 700 kDa, 500 kDa and 700 kDa, 600 kDa and 700 kDa, or 500 kDa and 600 kDa. In some embodiments, ultrafiltration yields as retentate a recombinant protein slurry in water, and a permeate comprising the impurities. Suitable conditions for ultrafiltration (e.g., membranes, temperature, volume replacement) can be determined using methods known in the art geared towards maximizing permeate density. In some embodiments, the ultrafiltration provides a rententate that has a density of between 1 g/mL and 30 g/mL. In some embodiments, ultrafiltration comprises a concentrating step that yields a concentrated retentate, followed by a diafiltration step that removes the impurities and yields the suspended protein slurry in water. In some such embodiments, the concentrated retentate has a concentration factor of between 2-fold and 12-fold volume reduction to starting volume. In some embodiments, the diafiltration provides a constant volume replacement of between 3-fold and 10-fold. Diafiltration is dilution process that involves removal or separation of components of a solution, such as salts, small molecules, proteins, solvents, and the like, based on the molecular size of the components via micro permeable filters.

Depending on the embodiment and the type of impurity to be removed, methods of removing impurities may differ. Removing lipid impurities from the solution comprising the solubilized silk protein can be accomplished by methods known in the art. Non-limiting examples of such methods include absorption to charcoals or other absorption media that specifically bind lipids. Removing polysaccharide impurities from the isolated recombinant protein can be accomplished by methods known in the art. Non-limiting examples of such methods include treatment with enzymes that hydrolyze polysaccharides followed by removal of the small sugars produced by ultrafiltration. Non-limiting examples of such enzymes include glucanase, lyticase, mannase, and chitinase.

Quantification

In some embodiments, the isolated recombinant spider silk protein is at least 60%, at least 65%, at least 70%, at least 75%, at least 80%, at least 85%, at least 90%, at least 95%, or at least 99% full-length recombinant spider silk protein.

In some embodiments, the purity of the isolated recombinant spider silk protein is 5-10%, 10-15%, 15-20%, 20-25%, 25-30%, 30-35%, 35-40%, 45-50%, 50-55%, 55-60%, 60-65%, 65-70%, 70-75%, 75-80%, 80-85%, 85-90%, 90-95%, or 95-100%. In some embodiments, the purity of the isolated recombinant spider silk protein is at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

In some embodiments, the isolated recombinant spider silk protein comprises at least 60%, at least 65%, at least 70%, at least 75%, at least 80%, at least 85%, at least 90%, or at least 95% full-length recombinant spider silk protein.

In some embodiments, the full-length recombinant spider silk protein is measured or quantified. Any appropriate method may be used to measure or quantify the amount of full length recombinant protein, including, but not limited so, size exclusion chromatography (SEC), SDS-PAGE, immunoblot (Western blot), high performance liquid chromatography (HPLC), SEC HPLC, liquid chromatography-mass spectrometry (LC-MS), or fast protein liquid chromatography (FPLC), or any other appropriate method known in the art, or any combination thereof. In one embodiment, the amount of full-length recombinant spider silk protein is measured using a western blot. In another embodiment, the amount of full-length recombinant spider silk protein is measured using size exclusion chromatography (SEC).

Recombinant Spider Silk Compositions

Silk polypeptides come from a variety of sources, including bees, moths, spiders, mites, and other arthropods. Some organisms make multiple silk fibers with unique sequences, structural elements, and mechanical properties. For example, orb weaving spiders have six unique types of glands that produce different silk polypeptide sequences that are polymerized into fibers tailored to fit an environmental or lifecycle niche. The fibers are named for the gland they originate from and the polypeptides are labeled with the gland abbreviation (e.g. “Ma”) and “Sp” for spidroin (short for spider fibroin). In orb weavers, these types include Major Ampullate (MaSp, also called dragline), Minor Ampullate (MiSp), Flagelliform (Flag), Aciniform (AcSp), Tubuliform (TuSp), and Pyriform (PySp). This combination of polypeptide sequences across fiber types, domains, and variation amongst different genus and species of organisms leads to a vast array of potential properties that can be harnessed by commercial production of the recombinant fibers. To date, the vast majority of the work with recombinant silks has focused on the Major Ampullate Spidroins (MaSp).

U.S. Pat. No. 9,963,554, “Methods and Compositions for Synthesizing Improved Silk Fibers,” incorporated herein by reference, discloses compositions for synthetic block copolymers, recombinant microorganisms for their production, and synthetic fibers comprising the proteins. US Patent Publication 2019/0100740, published Apr. 4, 2019, and titled “Modified Strains for the Production of Recombinant Silk,” incorporated herein by reference in its entirety, discloses engineered Pichia pastoris cells selected or genetically engineered to reduce degradation of recombinant proteins expressed by the yeast cells, and to methods of cultivating yeast cells for the production of useful compounds.

Several types of native spider silks have been identified. The mechanical properties of each natively spun silk type are believed to be closely connected to the molecular composition of that silk. See, e.g., Garb, J. E., et al., Untangling spider silk evolution with spidroin terminal domains, BMC Evol. Biol., 10:243 (2010); Bittencourt, D., et al., Protein families, natural history and biotechnological aspects of spider silk, Genet. Mol. Res., 11:3 (2012); Rising, A., et al., Spider silk proteins: recent advances in recombinant production, structure-function relationships and biomedical applications, Cell. Mol. Life Sci., 68:2, pg. 169-184 (2011); and Humenik, M., et al., Spider silk: understanding the structure-function relationship of a natural fiber, Prog. Mol. Biol. Transl. Sci., 103, pg. 131-85 (2011). For example:

Aciniform (AcSp) silks tend to have high toughness, a result of moderately high strength coupled with moderately high extensibility. AcSp silks are characterized by large block (“ensemble repeat”) sizes that often incorporate motifs of poly serine and GPX. Tubuliform (TuSp or Cylindrical) silks tend to have large diameters, with modest strength and high extensibility. TuSp silks are characterized by their poly serine and poly threonine content, and short tracts of poly alanine. Major Ampullate (MaSp) silks tend to have high strength and modest extensibility. MaSp silks can be one of two subtypes: MaSp1 and MaSp2. MaSp1 silks are generally less extensible than MaSp2 silks, and are characterized by poly alanine, GX, and GGX motifs. MaSp2 silks are characterized by poly alanine, GGX, and GPX motifs. Minor Ampullate (MiSp) silks tend to have modest strength and modest extensibility. MiSp silks are characterized by GGX, GA, and poly A motifs, and often contain spacer elements of approximately 100 amino acids. Flagelliform (Flag) silks tend to have very high extensibility and modest strength. Flag silks are usually characterized by GPG, GGX, and short spacer motifs.

The properties of each silk type can vary from species to species, and spiders leading distinct lifestyles (e.g. sedentary web spinners vs. vagabond hunters) or that are evolutionarily older may produce silks that differ in properties from the above descriptions (for descriptions of spider diversity and classification, see Hormiga, G., and Griswold, C. E., Systematics, phylogeny, and evolution of orb-weaving spiders, Annu. Rev. Entomol. 59, pg. 487-512 (2014); and Blackedge, T. A. et al., Reconstructing web evolution and spider diversification in the molecular era, Proc. Natl. Acad. Sci. U.S.A., 106:13, pg. 5229-5234 (2009)). However, synthetic block copolymer polypeptides having sequence similarity and/or amino acid composition similarity to the repeat domains of native silk proteins can be used to manufacture on commercial scales consistent silk-like fibers that recapitulate the properties of corresponding natural silk fibers.

In some embodiments, the recombinant spider silks are a highly crystalline silk protein, a high beta sheet content silk protein, or a low solubility silk protein. In some embodiments, the recombinant spider silk protein has a solubility threshold of less than 90%, 80%, 70%, 60%, or 50% in a non-chaotropic solvent.

Silk Nucleotide and Peptide Sequences

A list of putative silk sequences can be compiled by searching GenBank for relevant terms, e.g. “spidroin” “fibroin” “MaSp”, and those sequences can be pooled with additional sequences obtained through independent sequencing efforts. Sequences are then translated into amino acids, filtered for duplicate entries, and manually split into domains (NTD, REP, CTD). In some embodiments, candidate amino acid sequences are reverse translated into a DNA sequence optimized for expression in Pichia (Komagataella) pastoris. The DNA sequences are each cloned into an expression vector and transformed into Pichia (Komagataella) pastoris. In some embodiments, various silk domains demonstrating successful expression and secretion are subsequently assembled in combinatorial fashion to build silk molecules capable of fiber formation.

Silk polypeptides are characteristically composed of a repeat domain (REP) flanked by non-repetitive regions (e.g., C-terminal and N-terminal domains). The repeat domain exhibits a hierarchical architecture. The repeat domain comprises a series of blocks (also called repeat units). The blocks are repeated, sometimes perfectly and sometimes imperfectly (making up a quasi-repeat domain), throughout the silk repeat domain. The length and composition of blocks varies among different silk types and across different species. Table 1 lists examples of block sequences from selected species and silk types, with further examples presented in Rising, A. et al., Spider silk proteins: recent advances in recombinant production, structure-function relationships and biomedical applications, Cell Mol. Life Sci., 68:2, pg 169-184 (2011); and Gatesy, J. et al., Extreme diversity, conservation, and convergence of spider silk fibroin sequences, Science, 291:5513, pg. 2603-2605 (2001). In some cases, blocks may be arranged in a regular pattern, forming larger macro-repeats that appear multiple times (usually 2-8) in the repeat domain of the silk sequence. Repeated blocks inside a repeat domain or macro-repeat, and repeated macro-repeats within the repeat domain, may be separated by spacing elements. Block sequences may comprise a glycine rich region followed by a polyA region. Short (˜1-10) amino acid motifs may appear multiple times inside of blocks. A subset of commonly observed motifs is depicted in FIG. 1. Blocks from different natural silk polypeptides can be selected without reference to circular permutation (i.e., identified blocks that are otherwise similar between silk polypeptides may not align due to circular permutation). Thus, for example, a “block” of SGAGG is, for the purposes of the methods and compositions described herein, the same as GSGAG and the same as GGSGA; they are all just circular permutations of each other. The particular permutation selected for a given silk sequence can be dictated by convenience (usually starting with a G) more than anything else. Silk sequences obtained from the NCBI database can be partitioned into blocks and non-repetitive regions.

TABLE 1
Block Sequences

| Species | Silk Type | SEQ ID NO | Representative Block Amino Acid Sequence |
|---|---|---|---|
| Aliatypus gulosus | Fibroin 1 | 1 | GAASSSSTIITTKSASASAAADASAAATASAASRSSANAAASAFAQSF |
| | | | SSILLESGYFCSIFGSSISSSYAAAIASAASRAAAESNGYTTHAYACA |
| | | | KAVASAVERVTSGADAYAYAQAISDALSHALLYTGRLNTANANSLASA |
| | | | FAYAFANAAAQASASSASAGAASASGAASASGAGSAS |
| Plectreurys | Fibroin 1 | 2 | GAGAGAGAGAGAGAGAGSGASTSVSTSSSSGSGAGAGAGSGAGSGAGA |
| tristis | | | GSGAGAGAGAGGAGAGFGSGLGLGYGVGLSSAQAQAQAQAAAQAQAQA |
| | | | QAQAYAAQAQAQAQAQAQAA |
| Plectreurys | Fibroin 4 | 3 | GAAQKQPSGESSVATASAAATSVTSGGAPVGKPGVPAPIFYPQGPLQQ |
| tristis | | | GPAPGPSNVQPGTSQQGPIGGVGGSNAFSSSFASALSLNRGFTEVISS |
| | | | ASATAVASAFQKGLAPYGTAFALSAASAAADAYNSIGSGANAFAYAQA |
| | | | FARVLYPLVQQYGLSSSAKASAFASAIASSFSSGTSGQGPSIGQQQPP |
| | | | VTISAASASAGASAAAVGGGQVGQGPYGGQQQSTAASASAAAATATS |
| Araneus | TuSp | 4 | GNVGYQLGLKVANSLGLGNAQALASSLSQAVSAVGVGASSNAYANAVS |
| getntnoides | | | NAVGQVLAGQGILNAANAGSLASSFASALSSSAASVASQSASQSQAAS |
| | | | QSQAAASAFRQAASQSASQSDSRAGSQSSTKTTSTSTSGSQADSRSAS |
| | | | SSASQASASAFAQQSSASLSSSSSFSSAFSSATSISAV |
| Argiope aurantia | TuSp | 5 | GSLASSFASALSASAASVASSAAAQAASQSQAAASAFSRAASQSASQS |
| | | | AARSGAQSISTTTTTSTAGSQAASQSASSAASQASASSFARASSASLA |
| | | | ASSSFSSAFSSANSLSALGNVGYQLGFNVANNLGIGNAAGLGNALSQA |
| | | | VSSVGVGASSSTYANAVSNAVGQFLAGQGILNAANA |
| Deinopis spinosa | TuSp | 6 | GASASAYASAISNAVGPYLYGLGLFNQANAASFASSFASAVSSAVASA |
| | | | SASAASSAYAQSAAAQAQAASSAFSQAAAQSAAAASAGASAGAGASAG |
| | | | AGAVAGAGAVAGAGAVAGASAAAASQAAASSSASAVASAFAQSASYAL |
| | | | ASSSAFANAFASATSAGYLGSLAYQLGLTTAYNLGLSNAQAFASTLSQ |
| | | | AVTGVGL |
| Nephila clavipes | TuSp | 7 | GATAASYGNALSTAAAQFFATAGLLNAGNASALASSFARAFSASAESQ |
| | | | SFAQSQAFQQASAFQQAASRSASQSAAEAGSTSSSTTTTTSAARSQAA |
| | | | SQSASSSYSSAFAQAASSSLATSSALSRAFSSVSSASAASSLAYSIGL |
| | | | SAARSLGIADAAGLAGVLARAAGALGQ |
| Argiope | Flag | 8 | GGAPGGGPGGAGPGGAGFGPGGGAGFGPGGGAGFGPGGAAGGPGGPGG |
| trifasciata | | | PGGPGGAGGYGPGGAGGYGPGGVGPGGAGGYGPGGAGGYGPGGSGPGG |
| | | | AGPGGAGGEGPVTVDVDVTVGPEGVGGGPGGAGPGGAGFGPGGGAGFG |
| | | | PGGAPGAPGGPGGPGGPGGPGGPGGVGPGGAGGYGPGGAGGVGPAGTG |
| | | | GFGPGGAGGFGPGGAGGFGPGGAGGFGPAGAGGYGPGGVGPGGAGGFG |
| | | | PGGVGPGGSGPGGAGGEGPVTVDVDVSV |
| Nephila clavipes | Flag | 9 | GVSYGPGGAGGPYGPGGPYGPGGEGPGGAGGPYGPGGVGPGGSGPGGY |
| | | | GPGGAGPGGYGPGGSGPGGYGPGGSGPGGYGPGGSGPGGYGPGGSGPG |
| | | | GYGPGGYGPGGSGPGGSGPGGSGPGGYGPGGTGPGGSGPGGYGPGGSG |
| | | | PGGSGPGGYGPGGSGPGGFGPGGSGPGGYGPGGSGPGGAGPGGVGPGG |
| | | | FGPGGAGPGGAAPGGAGPGGAGPGGAGPGGAGPGGAGPGGAGPGGAGG |
| | | | AGGAGGSGGAGGSGGTTIIEDLDITIDGADGPITISEELPISGAGGSG |
| | | | PGGAGPGGVGPGGSGPGGVGPGGSGPGGVGPGGSGPGGVGPGGAGGPY |
| | | | GPGGSGPGGAGGAGGPGGAYGPGGSYGPGGSGGPGGAGGPYGPGGEGP |
| | | | GGAGGPYGPGGAGGPYGPGGAGGPYGPGGEGGPYGP |
| Latrodectus | AcSp | 10 | GINVDSDIGSVTSLILSGSTLQMTIPAGGDDLSGGYPGGFPAGAQPSG |
| hesperus | | | GAPVDFGGPSAGGDVAAKLARSLASTLASSGVFRAAFNSRVSTPVAVQ |
| | | | LTDALVQKIASNLGLDYATASKLRKASQAVSKVRMGSDTNAYALAISS |
| | | | ALAEVLSSSGKVADANINQIAPQLASGIVLGVSTTAPQFGVDLSSINV |
| | | | NLDISNVARNMQASIQGGPAPITAEGPDFGAGYPGGAPTDLSGLDMGA |
| | | | PSDGSRGGDATAKLLQALVPALLKSDVFRAIYKRGTRKQVVQYVTNSA |
| | | | LQQAASSLGLDASTISQLQTKATQALSSVSADSDSTAYAKAFGLAIAQ |
| | | | VLGTSGQVNDANVNQIGAKLATGILRGSSAVAPRLGIDLS |
| Argiope | AcSp | 11 | GAGYTGPSGPSTGPSGYPGPLGGGAPFGQSGFGGSAGPQGGFGATGGA |
| trifasciata | | | SAGLISRVANALANTSTLRTVLRTGVSQQIASSVVQRAAQSLASTLGV |
| | | | DGNNLARFAVQAVSRLPAGSDTSAYAQAFSSALFNAGVLNASNIDTLG |
| | | | SRVLSALLNGVSSAAQGLGINVDSGSVQSDISSSSSFLSTSSSSASYS |
| | | | QASASSTS |
| Uloborus | AcSp | 12 | GASAADIATAIAASVATSLQSNGVLTASNVSQLSNQLASYVSSGLSST |
| diversus | | | ASSLGIQLGASLGAGFGASAGLSASTDISSSVEATSASTLSSSASSTS |
| | | | VVSSINAQLVPALAQTAVLNAAFSNINTQNAIRIAELLTQQVGRQYGL |
| | | | SGSDVATASSQIRSALYSVQQGSASSAYVSAIVGPLITALSSRGVVNA |
| | | | SNSSQIASSLATAILQFTANVAPQFGISIPTSAVQSDLSTISQSLTAI |
| | | | SSQTSSSVDSSTSAFGGISGPSGPSPYGPQPSGPTFGPGPSLSGLTGF |
| | | | TATFASSFKSTLASSTQFQLIAQSNLDVQTRSSLISKVLINALSSLGI |
| | | | SASVASSIAASSSQSLLSVSA |
| Euprosthenops | MaSp1 | 13 | GGQGGQGQGRYGQGAGSSAAAA |
| australis | | | |
| Tetragnatha | MaSp1 | 14 | GGLGGGQGAGQGGQQGAGQGGYGSGLGGAGQGASAAAAAAAA |
| kauaiensis | | | |
| Argiope aurantia | MaSp2 | 15 | GGYGPGAGQQGPGSQGPGSGGQQGPGGLGPYGPSAAAAAAAA |
| Deinopis spinosa | MaSp2 | 16 | GPGGYGGPGQQGPGQGQYGPGTGQQGQGPSGQQGPAGAAAAAAAAA |
| Nephila clavata | MaSp2 | 17 | GPGGYGLGQQGPGQQGPGQQGPAGYGPSGLSGPGGAAAAAAA |
| Deinopis | MiSp | 18 | GAGYGAGAGAGGGAGAGTGYGGGAGYGTGSGAGYGAGVGYGAGAGAGG |
| Spinosa | | | GAGAGAGGGTGAGAGGGAGAGYGAGTGYGAGAGAGGGAGAGAGAGAGA |
| | | | GAGAGSGAGAGYGAGAGYGAGAGAGGVAGAGAAGGAGAAGGAGAAGGA |
| | | | GAAGGAGAGAGAGSGAGAGAGGGARAGAGG |
| Latrodectus | MiSp | 19 | GGGYGRGQGAGAGVGAGAGAAAGAAAIARAGGYGQGAGGYGQGQGAGA |
| hesperus | | | AAGAAAGAGAGGYGQGAGGYGRGQGAGAGAGAGAGARGYGQGAGAGAA |
| | | | AGAAASAGAGGYGQGAGGYGQGQGAGAAAGAAASAGAGGYGQGAGGYG |
| | | | QGQGA |
| Nephila clavipes | MiSp | 20 | GAGAGGAGYGRGAGAGAGAAAGAGAGAAAGAGAGAGGYGGQGGYGAGA |
| | | | GAGAAAAAGAGAGGAAGYSRGGRAGAAGAGAGAAAGAGAGAGGYGGQG |
| | | | GYGAGAGAGAAAAAGAGSGGAGGYGRGAGAGAAAGAGAAAGAGAGAGG |
| | | | YGGQGGYGAGAGAAAAA |
| Nephilengys | MiSp | 21 | GAGAGVGGAGGYGSGAGAGAGAGAGAASGAAAGAAAGAGAGGAGGYGT |
| cruentata | | | GQGYGAGAGAGAGAGAGGAGGYGRGAGAGAGAGAGGAGGYGAGQGYGA |
| | | | GAGAGAAAAAGDGAGAGGAGGYGRGAGAGAGAGAAAGAGAGGAGGYGA |
| | | | GQGYGAGAGAGAAAGAGAGGAGGYGAGQGYGAGAGAGAAAAA |
| Uloborus | MiSp | 22 | GSGAGAGSGYGAGAGAGAGSGYGAGSSASAGSAINTQTVTSSTTTSSQ |
| diversus | | | SSAAATGAGYGTGAGTGASAGAAASGAGAGYGGQAGYGQGAGASARAA |
| | | | GSGYGAGAGAAAAAGSGYGAGAGAGAGSGYGAGAAA |
| Uloborus | MiSp | 23 | GAGAGYRGQAGYIQGAGASAGAAAAGAGVGYGGQAGYGQGAGASAGAA |
| diversus | | | AAAGAGAGRQAGYGQGAGASAGAAAAGAGAGRQAGYGQGAGASAGAAA |
| | | | AGADAGYGGQAGYGQGAGASAGAAASGAGAGYGGQAGYGQGAGASAGA |
| | | | AAAGAGAGYLGQAGYGQGAGASAGAAAGAGAGYGGQAGYGQGTGAAAS |
| | | | AAASSA |
| Araneus | MaSp1 | 24 | GGQGGQGGYGGLGSQGAGQGGYGAGQGAAAAAAAAGGAGGAGRGGLGA |
| ventricosus | | | GGAGQGYGAGLGGQGGAGQAAAAAAAGGAGGARQGGLGAGGAGQGYGA |
| | | | GLGGQGGAGQGGAAAAAAAAGGQGGQGGYGGLGSQGAGQGGYGAGQGG |
| | | | AAAAAAAAGGQGGQGGYGGLGSQGAGQGGYGGRQGGAGAAAAAAAA |
| Dolotnedes | MaSp1 | 25 | GGAGAGQGSYGGQGGYGQGGAGAATATAAAAGGAGSGQGGYGGQGGLG |
| tenebrosus | | | GYGQGAGAGAAAAAAAAAGGAGAGQGGYGGQGGQGGYGQGAGAGAAAA |
| | | | AAGGAGAGQGGYGGQGGYGQGGGAGAAAAAAAASGGSGSGQGGYGGQG |
| | | | GLGGYGQGAGAGAGAAASAAAA |
| Nephilengys | MaSp | 26 | GGAGQGGYGGLGGQGAGAAAAAAGGAGQGGYGGQGAGQGAAAAAASGA |
| cruentata | | | GQGGYEGPGAGQGAGAAAAAAGGAGQGGYGGLGGQGAGQGAGAAAAAA |
| | | | GGAGQGGYGGLGGQGAGQGAGAAAAAAGGAGQGGYGGQGAGQGAAAAA |
| | | | AGGAGQGGYGGLGSGQGGYGRQGAGAAAAAAAA |
| Nephilengys | MaSp | 27 | GGAGQGGYGGLGGQGAGAAAAAAGGAGQGGYGGQGAGQGAAAAAASGA |
| cruentata | | | GQGGYGGPGAGQGAGAAAAAAGGAGQGGYGGLGGQGAGQGAGAAAAAA |
| | | | GGAGQGGYGGQGAGQGAAAAAAGGAGQGGYGGLGSGQGGYGGQGAGAA |
| | | | AAAGGAGQGGYGGLGGQGAGQGAGAAAAAA |

Fiber-forming block copolymer polypeptides from the blocks and/or macro-repeat domains, according to certain embodiments, is described in International Publication No. WO/2015/042164, incorporated by reference. Natural silk sequences obtained from a protein database such as GenBank or through de novo sequencing are broken up by domain (N-terminal domain, repeat domain, and C-terminal domain). The N-terminal domain and C-terminal domain sequences selected for the purpose of synthesis and assembly into fibers include natural amino acid sequence information and other modifications described herein. The repeat domain is decomposed into repeat sequences containing representative blocks, usually 1-8 depending upon the type of silk, that capture critical amino acid information while reducing the size of the DNA encoding the amino acids into a readily synthesizable fragment. In some embodiments, a properly formed block copolymer polypeptide comprises at least one repeat domain comprising at least 1 repeat sequence, and is optionally flanked by an N-terminal domain and/or a C-terminal domain.

In some embodiments, a repeat domain comprises at least one repeat sequence. In some embodiments, the repeat sequence is 150-300 amino acid residues. In some embodiments, the repeat sequence comprises a plurality of blocks. In some embodiments, the repeat sequence comprises a plurality of macro-repeats. In some embodiments, a block or a macro-repeat is split across multiple repeat sequences.

In some embodiments, the repeat sequence starts with a Glycine, and cannot end with phenylalanine (F), tyrosine (Y), tryptophan (W), cysteine (C), histidine (H), asparagine (N), methionine (M), or aspartic acid (D) to satisfy DNA assembly requirements. In some embodiments, some of the repeat sequences can be altered as compared to native sequences. In some embodiments, the repeat sequences can be altered such as by addition of a serine to the C terminus of the polypeptide (to avoid terminating in F, Y, W, C, H, N, M, or D). In some embodiments, the repeat sequence can be modified by filling in an incomplete block with homologous sequence from another block. In some embodiments, the repeat sequence can be modified by rearranging the order of blocks or macrorepeats.

In some embodiments, non-repetitive N- and C-terminal domains can be selected for synthesis. In some embodiments, N-terminal domains can be by removal of the leading signal sequence, e.g., as identified by SignalP (Peterson, T. N., et. Al., SignalP 4.0: discriminating signal peptides from transmembrane regions, Nat. Methods, 8:10, pg. 785-786 (2011).

In some embodiments, the N-terminal domain, repeat sequence, or C-terminal domain sequences can be derived from Agelenopsis aperta, Aliatypus gulosus, Aphonopelma seemanni, Aptostichus sp. AS217, Aptostichus sp. AS220, Araneus diadematus, Araneus gemmoides, Araneus ventricosus, Argiope amoena, Argiope argentata, Argiope bruennichi, Argiope trifasciata, Atypoides riversi, Avicularia juruensis, Bothriocyrtum californicum, Deinopis Spinosa, Diguetia canities, Dolomedes tenebrosus, Euagrus chisoseus, Euprosthenops australis, Gasteracantha mammosa, Hypochilus thorelli, Kukulcania hibernalis, Latrodectus hesperus, Megahexura fulva, Metepeira grandiosa, Nephila antipodiana, Nephila clavata, Nephila clavipes, Nephila madagascariensis, Nephila pilipes, Nephilengys cruentata, Parawixia bistriata, Peucetia viridans, Plectreurys tristis, Poecilotheria regalis, Tetragnatha kauaiensis, or Uloborus diversus.

In some embodiments, the silk polypeptide nucleotide coding sequence can be operatively linked to an alpha mating factor nucleotide coding sequence. In some embodiments, the silk polypeptide nucleotide coding sequence can be operatively linked to another endogenous or heterologous secretion signal coding sequence. In some embodiments, the silk polypeptide nucleotide coding sequence can be operatively linked to a 3× FLAG nucleotide coding sequence. In some embodiments, the silk polypeptide nucleotide coding sequence is operatively linked to other affinity tags such as 6-8 His residues.

Secretion Signals

The amount of protein that is secreted from a cell varies significantly between proteins, and is dependent in part on the secretion signal that is operably linked to the protein in its nascent state. A number of secretion signals are known in the art, and some are commonly used for production of secreted recombinant proteins. Prominent among these is the secretion signal of the α-mating factor (αMF) of Saccharomyces cerevisiae, which consists of a N-terminal 19-amino-acid signal peptide (also referred to herein as pre-αMF(sc)) followed by a 70-amino-acid leader peptide (also referred to herein as pro-αMF(sc)). Inclusion of pro-αMF(sc) in the secretion signal of the αMF of Saccharomyces cerevisiae (also referred to herein as pre-αMF(sc)/pro-αMF(sc) has proven critical for achieving high secreted yields of proteins. Addition of pro-αMF(sc) or functional variants thereof to signal peptides other than pre-αMF(sc) has also been explored as a means of achieving secretion of recombinant proteins, but has shown variable degrees of effectiveness, increasing secretion for certain recombinant proteins in certain recombinant host cells but having no effect or decreasing secretion for other recombinant proteins.

The use of multiple distinct secretion signals can improve the secreted yields of recombinant proteins, as described in U.S. application Ser. No. 15/724,196. Compared to recombinant host cells that comprise multiple polynucleotide sequences encoding a recombinant protein operably linked to just one secretion signal (e.g., pre-αMF(sc)/pro-αMF(sc)), recombinant host cells that comprise the same number of polynucleotide sequences encoding the recombinant protein operably linked to at least 2 distinct secretion signals produce higher secreted yields of the recombinant protein. Without wishing to be bound by theory, the use of at least 2 distinct secretion signals may permit the recombinant host cell to engage distinct cellular secretory pathways to effect efficient secretion of the recombinant protein and thus prevent over-saturation of any one secretion pathway.

At least one of the distinct secretion signals comprises a signal peptide may be selected from Table 2 or 3 or is a functional variant that has an at least 80% amino acid sequence identity to a signal peptide selected from Table 2 or 3. In some embodiments, the functional variant is a signal peptide selected from Table 2 or 3 that comprises one or two substituted amino acids. In some such embodiments, the functional variant has an at least 85%, at least 90%, at least 95%, or at least 99% amino acid sequence identity to a signal peptide selected from Table 2 or 3. In some embodiments, the signal peptide mediates translocation of the nascent recombinant protein into the ER post-translationally (i.e., protein synthesis precedes translocation such that the nascent recombinant protein is present in the cell cytosol prior to translocating into the ER). In other embodiments, the signal peptide mediates translocation of the nascent recombinant protein into the ER co-translationally (i.e., protein synthesis and translocation into the ER occur simultaneously). An advantage of using a signal peptide that mediates co-translational translocation into the ER is that recombinant proteins prone to rapid folding are prevented from assuming conformations that hinder translocation into the ER and thus secretion.

TABLE 2
Secretion Signals

| Source Gene ID | Species | Name | SEQ ID NO | Sequence |
|---|---|---|---|---|
| PEP4 | Saccharomyces | pre- | 28 | MFSLKALLPLALLLVSANQVAA |
| | cerevisiae | PEP4(sc) | | |
| PAS_chr1-1_0130 | Pichia pastoris | pre | 29 | MSFSSNVPQLFLLLVLLTNIVSG |
| | | DSE4(pp) | | |
| PAS_chr3_0076 | Pichia pastoris | pre | 30 | MKLSTNLILAIAAASAVVSA |
| | | EPX1(pp) | | |
| P00698 | Gallus gallus | pre | 31 | MRSLLILVLCFLPLAALG |
| | | CLSP(gg) | | |

TABLE 3
Recombinant Secretion Signals

| Name | SEQ ID NO | Sequence |
|---|---|---|
| pre-αMF(sc)/pro- | 33 | MRFPSIFTAVLFAASSALAAPVNTTTEDETAQIPAEAVIGYLDLEGDFDV |
| αMF(sc) | | AVLPFSNSTNNGLLFINTTIASIAAKEEGVSLDKREAEA |
| pre-αMF(sc)/*pro- | 34 | MRFPSIFTAVLFAASSALAAPVNTTTEDETAQIPAEAVIGYSDLEGDFDV |
| αMF(sc) | | AVLPFSNSTNNGLLFINTTIASIAAKEEGVSLEKREAEA |
| pre-PEP4(sc)/ | 35 | MFSLKALLPLALLLVSANQVAAAPVNTTTEDETAQIPAEAVIGYSDLEGD |
| *pro-αMF(sc) | | FDVAVLPFSNSTNNGLLFINTTIASIAAKEEGVSLEKREAEA |
| pre-DSE4(pp)/ | 36 | MSFSSNVPQLFLLLVLLTNIVSGAPVNTTTEDETAQIPAEAVIGYSDLEG |
| *pro-αMF(sc) | | DFDVAVLPFSNSTNNGLLFINTTIASIAAKEEGVSLEKREAEA |
| pre-EPX1 (pp)/ | 37 | MKLSTNLILAIAAASAVVSAAPVNTTTEDETAQIPAEAVIGYSDLEGDFD |
| *pro-αMF(sc) | | VAVLPFSNSTNNGLLFINTTIASIAAKEEGVSLEKREAEA |
| pre-CLSP(gg)/ | 38 | MRSLLILVLCFLPLAALGAPVNTTTEDETAQIPAEAVIGYSDLEGDFDVA |
| *pro-αMF(sc) | | VLPFSNSTNNGLLFINTTIASIAAKEEGVSLEKREAEA |

Expression Vectors

The expression vectors described herein can be produced following the teachings of the present specification in view of techniques known in the art. Sequences, for example vector sequences or sequences encoding transgenes, can be commercially obtained from companies such as Integrated DNA Technologies, Coralville, Iowa or DNA 2.0, Menlo Park, Calif. Exemplified herein are expression vectors that direct high-level expression of the chimeric silk polypeptides.

Another standard source for the polynucleotides described herein is polynucleotides isolated from an organism (e.g., bacteria), a cell, or selected tissue. Nucleic acids from the selected source can be isolated by standard procedures, which typically include successive phenol and phenol/chloroform extractions followed by ethanol precipitation. After precipitation, the polynucleotides can be treated with a restriction endonuclease which cleaves the nucleic acid molecules into fragments. Fragments of the selected size can be separated by a number of techniques, including agarose or polyacrylamide gel electrophoresis or pulse field gel electrophoresis (Care et al. (1984) Nuc. Acid Res. 12:5647-5664; Chu et al. (1986) Science 234:1582; Smith et al. (1987) Methods in Enzymology 151:461), to provide an appropriate size starting material for cloning.

Another method of obtaining the nucleotide components of the expression vectors or constructs is PCR. General procedures for PCR are taught in MacPherson et al., PCR: A PRACTICAL APPROACH, (IRL Press at Oxford University Press, (1991)). PCR conditions for each application reaction may be empirically determined. A number of parameters influence the success of a reaction. Among these parameters are annealing temperature and time, extension time, Mg2+ and ATP concentration, pH, and the relative concentration of primers, templates and deoxyribonucleotides. Exemplary primers are described below in the Examples. After amplification, the resulting fragments can be detected by agarose gel electrophoresis followed by visualization with ethidium bromide staining and ultraviolet illumination.

Another method for obtaining polynucleotides is by enzymatic digestion. For example, nucleotide sequences can be generated by digestion of appropriate vectors with suitable recognition restriction enzymes. Restriction cleaved fragments may be blunt ended by treating with the large fragment of E. coli DNA polymerase I (Klenow) in the presence of the four deoxynucleotide triphosphates (dNTPs) using standard techniques.

Polynucleotides are inserted into suitable backbones, for example, plasmids, using methods well known in the art. For example, insert and vector DNA can be contacted, under suitable conditions, with a restriction enzyme to create complementary or blunt ends on each molecule that can pair with each other and be joined with a ligase. Alternatively, synthetic nucleic acid linkers can be ligated to the termini of a polynucleotide. These synthetic linkers can contain nucleic acid sequences that correspond to a particular restriction site in the vector DNA. Other means are known and available in the art. A variety of sources can be used for the component polynucleotides.

In some embodiments, expression vectors containing an R, N, or C sequence are transformed into a host organism for expression and secretion. In some embodiments, the expression vectors comprise a secretion signal. In some embodiments, the expression vector comprises a terminator signal. In some embodiments, the expression vector is designed to integrate into a host cell genome and comprises: regions of homology to the target genome, a promoter, a secretion signal, a tag (e.g., a Flag tag), a termination/polyA signal, a selectable marker for Pichia, a selectable marker for E. coli, an origin of replication for E. coli, and restriction sites to release fragments of interest.

Host Cell Transformants

Host cells transformed with nucleic acid molecules or vectors that express spider silk polypeptides, and descendants thereof, are provided. These cells can also carry the nucleic acid sequences on vectors, which may but need not be freely replicating vectors. In other embodiments, the nucleic acids have been integrated into the genome of the host cells.

In some embodiments, microorganisms or host cells that enable the large-scale production of block copolymer polypeptides include a combination of: 1) the ability to produce large (>75 kDa) polypeptides, 2) the ability to secrete polypeptides outside of the cell and circumvent costly downstream intracellular purification, 3) resistance to contaminants (such as viruses and bacterial contaminations) at large-scale, and 4) the existing know-how for growing and processing the organism is large-scale (1-2000 m3) bioreactors.

A variety of host organisms can be engineered/transformed to comprise a block copolymer polypeptide expression system. Preferred organisms for expression of a recombinant silk polypeptide include yeast, fungi, gram-positive, and gram-negative bacteria. In certain embodiments, the host organism is Arxula adeninivorans, Aspergillus aculeatus, Aspergillus awamori, Aspergillus ficuum, Aspergillus fumigatus, Aspergillus japonicus, Aspergillus nidulans, Aspergillus niger, Aspergillus oryzae, Aspergillus sojae, Aspergillus tubigensis, Bacillus alkalophilus, Bacillus amyloliquefaciens, Bacillus anthracis, Bacillus brevis, Bacillus circulans, Bacillus coagulans, Bacillus lautus, Bacillus lentus, Bacillus licheniformis, Bacillus methanolicus, Bacillus stearothermophilus, Bacillus subtilis, Bacillus thuringiensis, Candida boidinii, Chrysosporium lucknowense, Escherichia coli, Fusarium graminearum, Fusarium venenatum, Kluyveromyces lactis, Kluyveromyces marxianus, Myceliopthora thermophila, Neurospora crassa, Ogataea polymorpha, Penicillium camemberti, Penicillium canescens, Penicillium chrysogenum, Penicillium emersonii, Penicillium funiculosum, Penicillium griseoroseum, Penicillium purpurogenum, Penicillium roqueforti, Phanerochaete chrysosporium, Pichia angusta, Pichia methanolica, Pichia (Komagataella) pastoris, Pichia polymorpha, Pichia stipitis, Rhizomucor miehei, Rhizomucor pusillus, Rhizopus arrhizus, Streptomyces lividans, Saccharomyces cerevisiae, Schwanniomyces occidentalis, Trichoderma harzianum, Trichoderma reesei, or Yarrowia lipolytica.

In preferred aspects, the methods provide culturing host cells for direct product secretion for easy recovery without the need to extract biomass. In some embodiments, the block copolymer polypeptides are secreted directly into the medium for collection and processing.

Engineered Host Cell Lines

The methylotrophic yeast Pichia pastoris is widely used in the production of recombinant proteins. P. pastoris grows to high cell density, provides tightly controlled methanol-inducible trans gene expression and efficiently secretes heterologous proteins in defined media. However, during culture of a strain of P. pastoris, recombinantly expressed proteins may be degraded before they can be collected, resulting in a mixture of proteins that includes fragments of recombinantly expressed proteins and a decreased yield of full-length recombinant proteins. Another widely used cell line for recombinant protein production is the bacteria Escherichia coli. However, during culture of a strain of E. coli, recombinantly expressed proteins may be insoluble, resulting in poor isolation and decreased yield of recombinant proteins.

In some embodiments, the modified strains with reduced protease activity described herein recombinantly express a silk-like polypeptide sequence. In some embodiments, the silk-like polypeptide sequences are 1) block copolymer polypeptide compositions generated by mixing and matching repeat domains derived from silk polypeptide sequences and/or 2) recombinant expression of block copolymer polypeptides having sufficiently large size (approximately 40 kDa) to form useful solids or fibers by secretion from an industrially scalable microorganism. Large (approximately 40 kDa to approximately 100 kDa) block copolymer polypeptides engineered from silk repeat domain fragments, including sequences from almost all published amino acid sequences of spider silk polypeptides, can be expressed in the modified microorganisms described herein. In some embodiments, silk polypeptide sequences are matched and designed to produce highly expressed and secreted polypeptides capable of solids or fiber formation. In some embodiments, knock-out of protease genes or reduction of protease activity in the host modified strain reduces degradation of the silk like polypeptides.

In some embodiments, to attenuate a protease activity in Pichia pastoris, the genes encoding these enzymes are inactivated or mutated to reduce or eliminate activity. This can be done through mutations or insertions into the gene itself of through modification of a gene regulatory element. This can be achieved through standard yeast genetics techniques. Examples of such techniques include gene replacement through double homologous recombination, in which homologous regions flanking the gene to be inactivated are cloned in a vector flanking a selectable maker gene (such as an antibiotic resistance gene or a gene complementing an auxotrophy of the yeast strain).

Alternatively, the homologous regions can be PCR-amplified and linked through overlapping PCR to the selectable marker gene. Subsequently, such DNA fragments are transformed into Pichia pastoris through methods known in the art, e.g., electroporation. Transformants that then grow under selective conditions are analyzed for the gene disruption event through standard techniques, e.g. PCR on genomic DNA or Southern blot. In an alternative experiment, gene inactivation can be achieved through single homologous recombination, in which case, e.g. the 5′ end of the gene's ORF is cloned on a promoterless vector also containing a selectable marker gene. Upon linearization of such vector through digestion with a restriction enzyme only cutting the vector in the target-gene homologous fragment, such vector is transformed into Pichia pastoris. Integration at the target gene site is confirmed through PCR on genomic DNA or Southern blot. In this way, a duplication of the gene fragment cloned on the vector is achieved in the genome, resulting in two copies of the target gene locus: a first copy in which the ORF is incomplete, thus resulting in the expression (if at all) of a shortened, inactive protein, and a second copy which has no promoter to drive transcription.

Alternatively, transposon mutagenesis is used to inactivate the target gene. A library of such mutants can be screened through PCR for insertion events in the target gene.

The functional phenotype (i.e., deficiencies) of an engineered/knockout strain can be assessed using techniques known in the art. For example, a deficiency of an engineered strain in protease activity can be ascertained using any of a variety of methods known in the art, such as an assay of hydrolytic activity of chromogenic protease substrates, band shifts of substrate proteins for the selected protease, among others.

Attenuation of a protease activity described herein can be achieved through mechanisms other than a knockout mutation. For example, a desired protease can be attenuated via amino acid sequence changes by altering the nucleic acid sequence, placing the gene under the control of a less active promoter, down-regulation, expressing interfering RNA, ribozymes or antisense sequences that target the gene of interest, or through any other technique known in the art. In preferred strains, the protease activity of proteases encoded at PAS_chr4_0584 (YPS1-1) and PAS_chr3_1157 (YPS1-2) is attenuated by any of the methods described above. In some aspects, methylotrophic yeast strains, especially Pichia pastoris strains, wherein a YPS1-1 and a YPS1-2 gene have been inactivated are described. In some embodiments, additional protease encoding genes may also be knocked-out in accordance with the methods provided herein to further reduce protease activity of a desired protein product expressed by the strain.

In some embodiments, the P. pastoris strains disclosed herein have been modified to express a silk-like polypeptide. Methods of manufacturing preferred embodiments of silk-like polypeptides are provided in WO 2015/042164, especially at Paragraphs 114-134, incorporated herein by reference. Disclosed therein are synthetic proteinaceous copolymers based on recombinant spider silk protein fragment sequences derived from MaSp2, such as from the species Argiope bruennichi. Silk-like polypeptides are described that include two to twenty repeat units, in which a molecular weight of each repeat unit is greater than about 20 kDa. Within each repeat unit of the copolymer are more than about 60 amino acid residues that are organized into a number of “quasi-repeat units.” In some embodiments, the repeat unit of a polypeptide described in this disclosure has at least 95% sequence identity to a MaSp2 dragline silk protein sequence.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B(1992).

### Example 1: Calcium Salt Extraction

Highly crystalline silks form aggregates in solution, resulting in decreased solubility and thus decreased recovery from host cells during production. Thus, improved methods of solubilizing such crystalline silks are required. The method described in these examples is to use calcium salts and an alcohol to increase solubility of the silk protein.

Materials and Methods

Multiple calcium salts were used to extract the UDMisp64k protein, also referred to as P0 (representative block amino acid sequence shown in SEQ ID NO. 23), to identify the optimal calcium salt. P0 is an exemplary highly crystalline silk protein. E. coli transformed with an expression vector containing the P0 silk gene fused to a 6×His tag (6 histidines appended to the c-terminus of P0 with a glycine linker (GGGGG-HHHHHH)) were grown in a Terrific Broth, a defined minimal salt media, with chloramphenicol. P0 expression was induced with IPTG after 24 hours of fermentation. The E. coli was harvest after 16 hours of protein induction. The E. coli was lysed by passing the LB broth and cells trough a microfluidizer (Microfluidics LM10) in a single pass at 14,0000 PSI. The lysate was pelleted via centrifugation at 15,000×g in an Eppendorf table top centrifuge. The pellet containing the insoluble P0 was retained and the supernatant discarded.

20% (w/v) solutions of calcium chloride (CaCl₂), calcium nitrate (Ca(NO₃)₂ or CaNit), and calcium thiocyanate (C₂CaN₂S2 or Ca(SCN)₂ or CaSCN) in methanol were each prepared. 100 mg of the cell lysate pellet was added to 1 mL of each calcium salt/methanol (CaMeOH) solution. The cell lysate pellets were resuspended and incubated in each of the CaMeOH solutions for 1 hour at room temperature. Non-solubilized material was re-pelleted via centrifugation (15,000×g). The supernatants were retained and analyzed via SDS-PAGE in a Bis/Tris buffer and immunoblotted. P0 protein was visualized using an anti-His antibody.

Results

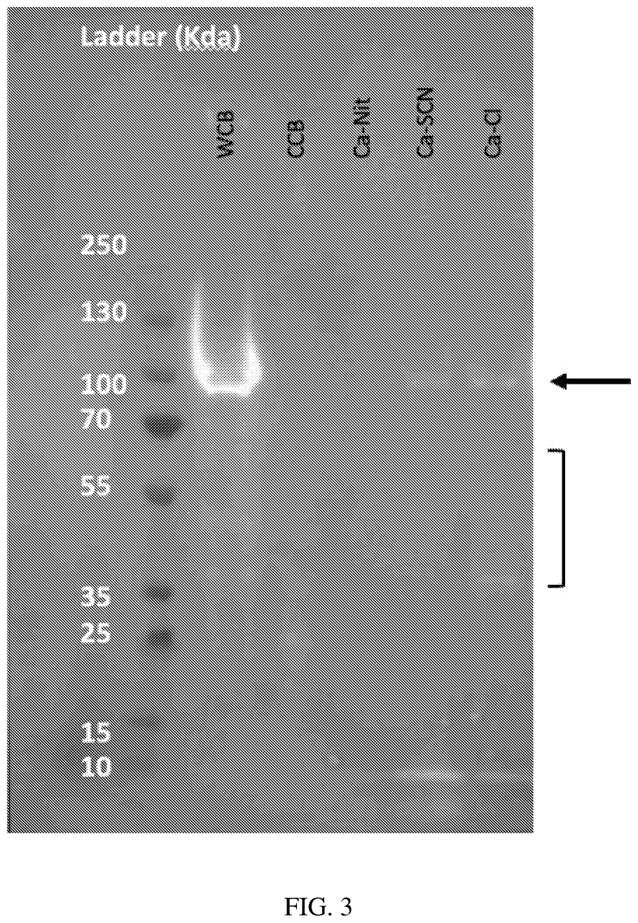

P0 monomer ran slightly higher than its molecular weight in the Bis/Tris Gels for Westerns. The P0 used in this example is 64 kDa, however it generally appears between the 70 and 100 kDa marker on SDS-PAGE gels. In this case, the protein ran at 100 kDa. Whole cell broth (WCB) was extracted with 5M guanidine thiocyanate, while clarified cell broth (CCB) was extracted with no solvents and served as a control. P0 protein monomers were observed in the supernatant fraction after incubation with solutions containing calcium thiocyanate (CaSCN) and calcium chloride (CaCl₂), as indicated by the protein band at 100 kDa (FIG. 3, as indicated by the arrow). However, no bands were observed in the calcium nitrate (CaNit) lane. Without intending to be bound by any particular theory, it is proposed that Ca—SCN may have higher specificity towards the full length P0 as no other bands below or above were visible. A band of similar intensity was observed in the CaCl₂ lane as well, along with smaller anti-His tagged species, possibly fragments of P0 (bands at about 55 kDa, 50 kDa, and 37 kDa, indicated by bracket).

### Example 2: Alcohol Extraction

The selection of the alcohol was investigated to determine the optimal extraction conditions. First, insoluble P0 was incubated with CaCl₂ in water or in methanol, to determine the requirement to include an alcohol solvent. Next, ethanol and isopropanol were substituted as the primary solvent. Finally, water was introduced along with methanol as the solvent, to reduce the volatility of the process.

Materials and Methods

P0 was expressed in E. coli cells as described in Example 1. Cells were lysed using a microfluidizer and the insoluble material was pelleted via centrifugation. Solutions with different concentrations of CaCl₂ in different solvents were made as shown in Table 4.

TABLE 4

| Condition # | CaCl₂ (M) | Solvent |
|---|---|---|
| 1 | 2 | Water |
| 2 | 3 | Water |
| 3 | 4 | Water |
| 4 | 1 | Methanol (MeOH) |
| 5 | 1.5 | Methanol (MeOH) |
| 6 | 2 | Methanol (MeOH) |
| 7 | 2 | Ethanol (EtOH) |
| 8 | 2 | 25% MeOH in water |
| 9 | 2 | 50% MeOH in water |
| 10 | 2 | 75% MeOH in water |

100 mg of insoluble cell material was added to 1 ml of each solution and resuspended via pipetting. Samples in solution conditions 1-6 were incubated at room temperature (about 22° C.) for 1 hour. Parallel samples of solution conditions 1-6 were made and incubated at 55° C. for 1 hour in a heating block (Benchmark Scientific BSH1002). Samples treated with solution conditions 7-10 were incubated at 55° C. for 1 hour in a heating block. After incubation, samples were pelleted via centrifugation. The supernatants containing the solubilized P0 protein collected and analyzed via an enzyme-linked immunosorbent assay (ELISA) for the His tag.

Results

ELISA results for the sample treated with solution conditions 1-6 are shown in Table 5 as a percentage of recovered P0 at 22° C. and 55° C. P0 yield quantitation was determined by ELISA using the following equation: (P0 in extract)/(P0 in WCB)=(P0 Extraction Yield). The symbol * indicates that P0 yield was undetectable by ELISA.

TABLE 5

| | 1 hour | | |
|---|---|---|---|
| | Extraction | P0 Yield % | |
| Condition # | Conditions | 22° C. | 55° C. |
| 1 | 2M CaCl₂ in Water | * | * |
| 2 | 3M CaCl₂ in Water | * | * |
| 3 | 4M CaCl₂ in Water | 1% | 3% |
| 4 | 1M CaCl₂ in MeOH | * | 3% |
| 5 | 1.5M CaCl₂ in MeOH | * | 4% |
| 6 | 2M CaCl₂ in MeOH | 18% | 51% |

P0 was undetectable by ELISA in all concentrations of CaCl₂ under 4M in aqueous and 2M in methanol at 22° C. 4M CaCl₂ in water yielded 1% P0, which increased to 3× to 3% with the addition of heat. 2M CaCl₂ in methanol also exhibited similar yield increases of 3× when the temperature was higher.

ELISA results of samples treated under solution conditions 7-10, and heat-treated condition 6, are shown in Table 6.

TABLE 6

| Extraction Conditions | |
|---|---|
| (1 hr @ 55 C.) | P0 Yield % |
| 2M CaCl₂ MeOH | 51% |
| 2M CaCl₂ EtOH | 5% |
| 2M CaCl₂ 25% MeOH | * |
| 75% Water | |
| 2M CaCl₂ 50% MeOH | * |
| 50% water | |
| 2M CaCl₂ 75% MeOH | 4% |
| 25% water | |

2M calcium chloride in ethanol did not extract P0 as well as in methanol. The yield was 10× lower under the same extraction conditions (5% in EtOH compared to 51% in MeOH).

Without intending to be bound by any particular theory, it is proposed that water negatively affected P0 extraction. P0 yield was as low as 4% when the solution contained only 25% water and 75% methanol, and had no measurable yield as the water content increased to 50% or 75%.

### Example 3: Incubation Time and Temperature

The temperature of the extraction was altered, to determine the optimal temperature for maximal extraction while minimizing the extraction time. Agitation of the samples was also introduced. Lowering the temperature along with continuous mixing was investigated as a more scalable process scenario.

Materials and Methods

P0 was expressed in E. coli cells as described in Example 1. Cells were lysed using a microfluidizer and the insoluble material was pelleted via centrifugation. 1 ml of a 2M CaCl₂ solution in methanol was added to 100 mg of the insoluble cell material, which was resuspended via pipetting. 12 aliquots were made. 6 aliquots were incubated at 35° C. with agitation for 0, 15, 30, 60, 120, and 240 min. The remaining 6 aliquots were incubated at 55° C. with agitation for 0, 5, 15, 30, 60, and 120 min. At each time point the samples were removed and centrifuged at 15,000×g in a benchtop centrifuge (Eppendorf 5415D). The supernatants containing the solubilized P0 protein collected and analyzed via ELISA for the His tag.

Results

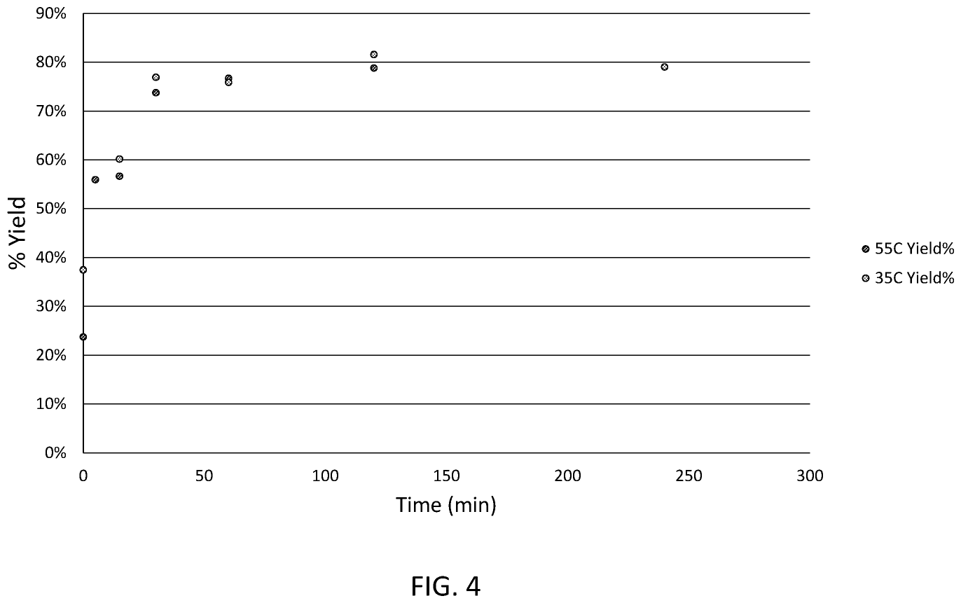

The extraction results are shown in FIG. 4. The amount of extracted P0 protein was substantially similar at each time point of the samples incubated at 35° C. compared to 55° C. Both extraction temperatures reaching peak extraction at 30 min. With the addition of continuous mixing during extraction, the maximum yield increased from approximately 50% to 80%. Yield percentages for each condition are shown in Table 7.

TABLE 7

| Time | 35° C. Yield % | 55° C. Yield % |
|---|---|---|
| 0 | 37 | 24 |
| 5 | — | 56 |
| 15 | 60 | 57 |
| 30 | 77 | 74 |
| 60 | 76 | 77 |
| 120 | 82 | 79 |
| 240 | 79 | — |

Thus, incubation at 35° C. was as effective as incubation at 55° C. In addition, agitation or mixing during incubation significantly improved P0 recovery.

### Example 4: Extraction Volume

To further improve production scalability, reducing the volume of solution used during extraction was explored. The volume of 2M calcium chloride solution was decreased by half to extract P0 from the insoluble pellet.

Materials and Methods

P0 was expressed in E. coli cells as described in Example 1. Cells were lysed using a microfluidizer and the insoluble material was pelleted via centrifugation. Insoluble pellets were resuspended in 0.5 ml or 1 ml of a 2M CaCl₂) solution in methanol. Samples were incubated at 35° C. for 1 hr with agitation. After incubation, the samples were pelleted via centrifugation and the supernatant retained. P0 in the supernatant was analyzed via ELISA and size exclusion chromatography (SEC). SEC was used to determine the relative amount of full length P0 in the samples.

Results

Yields of P0 in the 1 ml and 0.5 ml samples are shown in Table 8 below.

TABLE 8

| Volume of 2M | Yield % | Full Length |
|---|---|---|
| CaMeOH | (ELISA) | (SEC) |
| 0.5 mL | 70% | 22.05% |
| 1 mL | 78% | 19.88% |

In both samples, the yield was similar, indicating that the sample volume could be decreased and still result in efficient extraction of P0 protein. The 0.5 ml sample equates roughly to a mass ratio of 7:1 2M calcium methanol solution to pellet as compared to a 14:1 ratio in the 1 ml sample. The benefit of decreasing the extraction volume is worth the decrease in yield.

In addition, the amount of full length P0 in both samples was substantially similar (approx. 22% in the 0.5 ml sample compared to approx. 20% in the 1 ml sample), so purity of the recovered P0 was uncompromised.

### Example 5: P0 Powder Recovery

The P0 protein was recovered from the calcium salt and methanol solution.

Materials and Methods

To leverage the poor solubility of P0, water was added at a mass ratio of 1:2 water to extract to facilitate precipitation. A precipitate formed and was centrifuged at 4, 200×g for 15 min in a Beckman J-6 centrifuge. Full length P0 stably remained in solution in the supernatant. A sample of the water-precipitated supernatant was taken for SEC and ELISA analyses. The methanol in the retained supernatant was evaporated off in a rotary evaporator (Buchi Rotavapor R-210) set to 60° C. under vacuum. Once the methanol was evaporated, the sample was dialyzed against water in a dialysis cassette with a 20 kDa cut off (Slide-A-Lyzer Dialysis Cassete 20 kDa) to remove the calcium chloride. After dialysis a precipitate formed and was recovered as a pellet through centrifugation at 4,200×g for 15 min (Beckman J-6). The pellet was frozen at −80° C. and lyophilized (Labconco Freezone 4.5). The amount of full length P0 in solution and after lyophilization was determined via SEC and overall yield was determined by ELISA.

Results

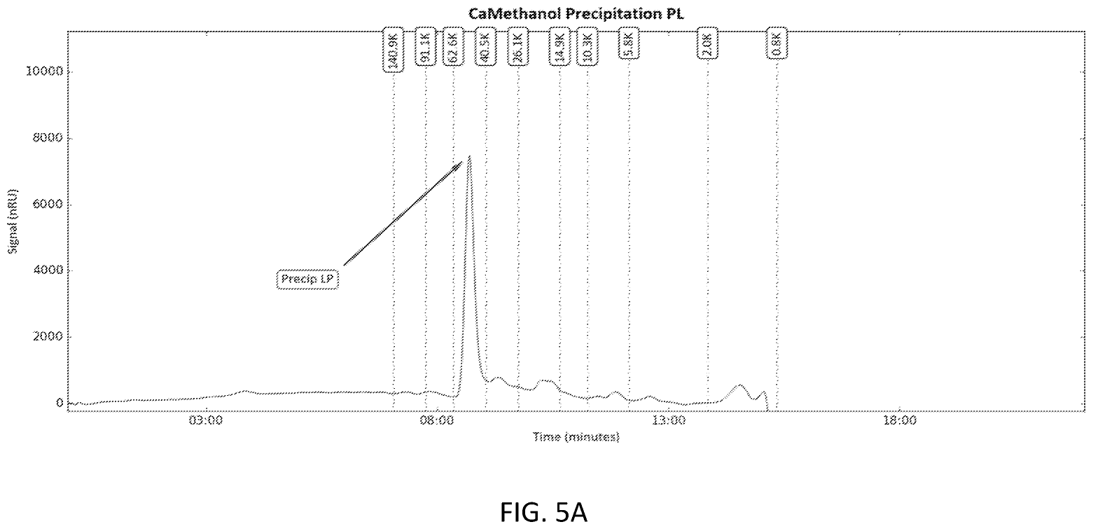

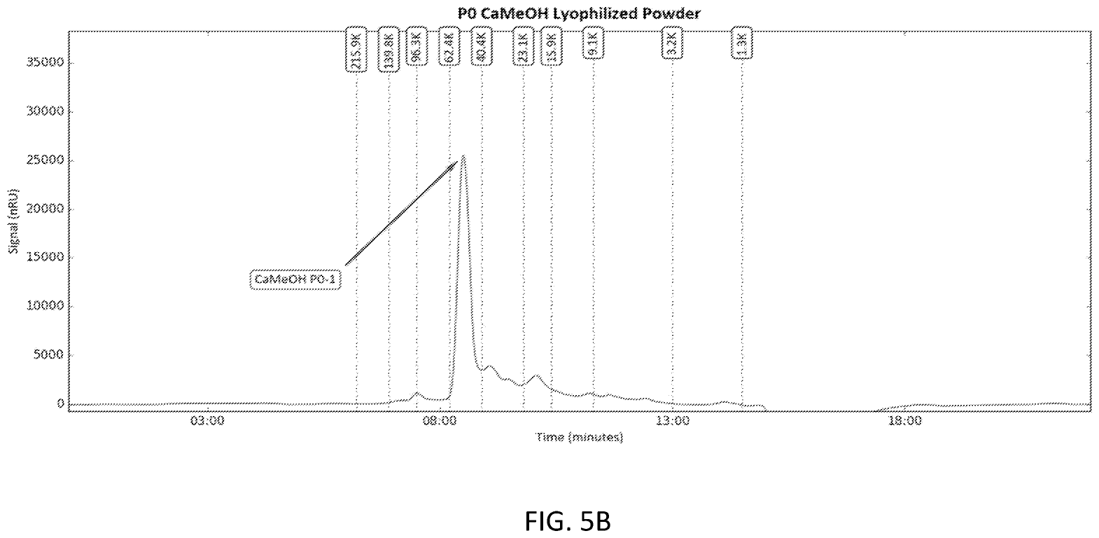

Water precipitation enriched the full length P0 in the extract from 20% to 50%, as quantified via SEC (FIG. 5A). The lyophilized P0 was 51% full length P0 monomer, as quantified via SEC (FIG. 5B).

Total P0 yield decreased by only 6% from 56% to 50% after water precipitation and lyophilization as quantified via ELISA.

Thus, water precipitation removed impurities while only minimally affecting the overall P0 protein yield.

### Example 6: High Throughput CaCl₂ in MeOH Extraction Screen

The methods described herein were performed on other silk proteins in a 96-well block CaCl₂ in MeOH Assay.

Materials and Methods

Silk proteins were expressed in E. coli cells as described in Example 1. Cell pellets were sonicated and 2M CaCl₂ solution in methanol was added. The samples were mixed to resuspend the cell pellets. Samples were incubated at 35° C. for 1 hr with agitation. The samples were analyzed via ELISA and extraction efficiency (%) was reported relative to a 5 M GdnSCN, pH 11 extraction control. The estimated crystal volume fraction (CVF) was estimated by first assigning the residues to the crystal motifs. The crystal motifs are defined by any contiguous sequence of six or more residues comprised only of alanine, glycine, isoleucine, serine, threonine, or valine, and where no glycine can be adjacent to another glycine. The sum of the residues in the crystalline motifs was then divided by the total number of residues to calculate the estimated crystal volume fraction.

Results

Table 9 shows the estimated percent crystal volume fraction, percent water content, and percent CaCl₂ in MeOH extracted efficiency of various silk proteins, including the P0 protein. The water content required for extraction was dependent on the silk protein. Sensitivity to water content was also dependent on the silk protein. The lowest extraction efficiency was 72%.

TABLE 9

| | | Crystal | | Ex- |
|---|---|---|---|---|
| | | volume | | tracted |
| SEQ | | fraction | Water | Ef- |
| ID | | (CVF) | Content | ficiency |
| NO. | Protein | (%) | (%) | (%) |
| 39 | SG MiSp 121 k | 29 | 23 | 81 |
| 40 | LG MiSp_v1 73 k | 35 | 9 | 92 |
| 41 | LG MiSp_v1 73 k (His) | 35 | 18 | 106 |
| 42 | NC MaSp 68k1 | 36 | 18 | 99 |
| 43 | NC MiSp 81 k | 42 | 18 | 90 |
| 44 | LT MiSp_v2 94 k (Flag + His) | 46 | 5 | 77 |
| 45 | LT MiSp_v2 94 k | 46 | 23 | 88 |
| 46 | LG MiSp_v1 80 k (His) | 46 | 18 | 84 |
| 47 | LG MiSp_v1 80 k (Flag + His) | 46 | 23 | 90 |
| 48 | LH MiSp 58 k | 51 | 18 | 96 |
| 49 | LH MiSp_v1 80 k | 54 | 18 | 118 |
| 50 | LH MiSp_v1 80 k (His) | 54 | 23 | 93 |
| 51 | NCr MiSp 67k1 | 60 | 18 | 100 |
| 52 | LT MiSp_v1 71 k | 60 | 18 | 74 |
| 53 | UD MiSp 32 k | 63 | 18 | 109 |
| 54 | UD MiSp 64 k | 64 | 5 | 117 |
| 55 | UD MiSp 127 k | 64 | 9 | 79 |
| 56 | NC MiSp 35k1 | 66 | 14 | 72 |
| 57 | NC MiSp 63 k | 67 | 18 | 98 |
| 58 | UD MiSp 63 k | 79 | 23 | 94 |
| 59 | UD MiSp 31k2 | 80 | 18 | 109 |

### EQUIVALENTS

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

SEQUENCE LISTING

| SEQ ID | Name | Sequence |
|---|---|---|
| 39 | SGMiSp 12 | GGYGPGQRAGPGQGAGPGQGVGPGQGVGTGGQGTGASSAAAASAGTSGYGPGVSGYGPA |
| | 1k | QGAGPGGQGTGASSAAATSAGTSGYGPGYPGYGQGPASGPTADKYGPGIGRYAPGRSTT |
| | | STSAATASATTVNNGPQIGGYGPGQGIGPAATSAPGASGYGPGVSGYGPGQGAGPGQGA |
| | | GPWQGVGPGQGAGPGGQGSGASSEAAASAGTSAYGPGVSGYGPGQGAGPGGQGSGASSR |
| | | AAASAGTRGYGPGYPGYGQGPASGPTAAYKYGPGIGGYAPGRSTTSTSAATASATTVDI |
| | | GPQVGGYGPGQGIAPAAAAPGSSGYGPGVSGYGPGQGAGPGGQGTGASSAAAASAGTSG |
| | | YGSGVSGYGPGQGAGPGQGAGPGQGVGPGQGAGLGQGVGPGQGAGPGGQGSGASTAAAA |
| | | SAGTSGYAPGVSGYGPGQGAGPGQGAGPGQGAGPGQGVGPGQGAGPGGQGSGASSAAAA |
| | | GGYGPGQRAGPGQGAGPGQGVGPGQGVGTGGQGTGASSAAAASAGTSGYGPGVSGYGPA |
| | | QGAGPGGQGTGASSAAATSAGTSGYGPGYPGYGQGPASGPTADKYGPGIGRYAPGRSTT |
| | | STSAATASATTVNNGPQIGGYGPGQGIGPAATSAPGASGYGPGVSGYGPGQGAGPGQGA |
| | | GPWQGVGPGQGAGPGGQGSGASSEAAASAGTSAYGPGVSGYGPGQGAGPGGQGSGASSR |
| | | AAASAGTRGYGPGYPGYGQGPASGPTAAYKYGPGIGGYAPGRSTTSTSAATASATTVDI |
| | | GPQVGGYGPGQGIAPAAAAPGSSGYGPGVSGYGPGQGAGPGGQGTGASSAAAASAGTSG |
| | | YGSGVSGYGPGQGAGPGQGAGPGQGVGPGQGAGLGQGVGPGQGAGPGGQGSGASTAAAA |
| | | SAGTSGYAPGVSGYGPGQGAGPGQGAGPGQGAGPGQGVGPGQGAGPGGQGSGASSAAAA |
| | | GGYGPGQRAGPGQGAGPGQGVGPGQGVGTGGQGTGASSAAAASAGTSGYGPGVSGYGPA |
| | | QGAGPGGQGTGASSAAATSAGTSGYGPGYPGYGQGPASGPTADKYGPGIGRYAPGRSTT |
| | | STSAATASATTVNNGPQIGGYGPGQGIGPAATSAPGASGYGPGVSGYGPGQGAGPGQGA |
| | | GPWQGVGPGQGAGPGGQGSGASSEAAASAGTSAYGPGVSGYGPGQGAGPGGQGSGASSR |
| | | AAASAGTRGYGPGYPGYGQGPASGPTAAYKYGPGIGGYAPGRSTTSTSAATASATTVDI |
| | | GPQVGGYGPGQGIAPAAAAPGSSGYGPGVSGYGPGQGAGPGGQGTGASSAAAASAGTSG |
| | | YGSGVSGYGPGQGAGPGQGAGPGQGVGPGQGAGLGQGVGPGQGAGPGGQGSGASTAAAA |
| | | SAGTSGYAPGVSGYGPGQGAGPGQGAGPGQGAGPGQGVGPGQGAGPGGQGSGASSAAAA |
| 40 | LGMiSp_v1 | GGYTQRQNEVITTVSTTRQKTADYGQKQVSGASAAVSTSSAGGYTQGPGGYGPGQGAVA |
| | 73k | GGYGPGAGSYGAGAIDASGGYGQGAGTAAGASASAGAGAATGVGPGGYGQGLGGYGQSA |
| | | GQGAGGYRQGAGTAAGASASAGAGAATGVGPGGYGQGLGGYGQAAGQGAGGYGQGAGTA |
| | | TSTATGAGTGGYGRLAGGYGQGAGGYGQAAAGAAADATAGAGGYDRATGAFGPSTRRAA |
| | | GGSGLGAGTAPGAFSGSGAGGKGPGDYGSSQGASASSSAAAAASGGYTQRQNEVITTVS |
| | | TTRQKTADYGQKQVSGASAAVSTSSAGGYTQGPGGYGPGQGAVAGGYGPGAGSYGAGAI |
| | | DASGGYGQGAGTAAGASASAGAGAATGVGPGGYGQGLGGYGQSAGQGAGGYRQGAGTAA |
| | | GASASAGAGAATGVGPGGYGQGLGGYGQAAGQGAGGYGQGAGTATSTATGAGTGGYGRL |
| | | AGGYGQGAGGYGQAAAGAAADATAGAGGYDRATGAFGPSTRRAAGGSGLGAGTAPGAFS |
| | | GSGAGGKGPGDYGSSQGASASSSAAAAASGGYTQRQNEVITTVSTTRQKTADYGQKQVS |
| | | GASAAVSTSSAGGYTQGPGGYGPGQGAVAGGYGPGAGSYGAGAIDASGGYGQGAGTAAG |
| | | ASASAGAGAATGVGPGGYGQGLGGYGQSAGQGAGGYRQGAGTAAGASASAGAGAATGVG |
| | | PGGYGQGLGGYGQAAGQGAGGYGQGAGTATSTATGAGTGGYGRLAGGYGQGAGGYGQAA |
| | | AGAAADATAGAGGYDRATGAFGPSTRRAAGGSGLGAGTAPGAFSGSGAGGKGPGDYGSS |
| | | QGASASSSAAAAAS |
| 41 | LGMiSp_v1 | GGYTQRQNEVITTVSTTRQKTADYGQKQVSGASAAVSTSSAGGYTQGPGGYGPGQGAVA |
| | 73k | GGYGPGAGSYGAGAIDASGGYGQGAGTAAGASASAGAGAATGVGPGGYGQGLGGYGQSA |
| | | GQGAGGYRQGAGTAAGASASAGAGAATGVGPGGYGQGLGGYGQAAGQGAGGYGQGAGTA |
| | | TSTATGAGTGGYGRLAGGYGQGAGGYGQAAAGAAADATAGAGGYDRATGAFGPSTRRAA |
| | | GGSGLGAGTAPGAFSGSGAGGKGPGDYGSSQGASASSSAAAAASGGYTQRQNEVITTVS |
| | | TTRQKTADYGQKQVSGASAAVSTSSAGGYTQGPGGYGPGQGAVAGGYGPGAGSYGAGAI |
| | | DASGGYGQGAGTAAGASASAGAGAATGVGPGGYGQGLGGYGQSAGQGAGGYRQGAGTAA |
| | | GASASAGAGAATGVGPGGYGQGLGGYGQAAGQGAGGYGQGAGTATSTATGAGTGGYGRL |
| | | AGGYGQGAGGYGQAAAGAAADATAGAGGYDRATGAFGPSTRRAAGGSGLGAGTAPGAFS |
| | | GSGAGGKGPGDYGSSQGASASSSAAAAASGGYTQRQNEVITTVSTTRQKTADYGQKQVS |
| | | GASAAVSTSSAGGYTQGPGGYGPGQGAVAGGYGPGAGSYGAGAIDASGGYGQGAGTAAG |
| | | ASASAGAGAATGVGPGGYGQGLGGYGQSAGQGAGGYRQGAGTAAGASASAGAGAATGVG |
| | | PGGYGQGLGGYGQAAGQGAGGYGQGAGTATSTATGAGTGGYGRLAGGYGQGAGGYGQAA |
| | | AGAAADATAGAGGYDRATGAFGPSTRRAAGGSGLGAGTAPGAFSGSGAGGKGPGDYGSS |
| | | QGASASSSAAAAAS |
| 42 | NCMaSp 68 | GGAGQGGYGGLGGQGAGAAAAAAGGAGQGGYGGQGAGQGAAAAAASGAGQGGYEGPGAG |
| | k1 | QGAGAAAAAAGGAGQGGYGGLGGQGAGQGAGAAAAAAGGAGQGGYGGLGGQGAGQGAGA |
| | | AAAAAGGAGQGGYGGQGAGQGAAAAAAGGAGQGGYGGLGSGQGGYGRQGAGAAAAAAAA |
| | | GGAGQGGYGGLGGQGAGAAAAAAGGAGQGGYGGQGAGQGAAAAAASGAGQGGYEGPGAG |
| | | QGAGAAAAAAGGAGQGGYGGLGGQGAGQGAGAAAAAAGGAGQGGYGGLGGQGAGQGAGA |
| | | AAAAAGGAGQGGYGGQGAGQGAAAAAAGGAGQGGYGGLGSGQGGYGRQGAGAAAAAAAA |
| | | GGAGQGGYGGLGGQGAGAAAAAAGGAGQGGYGGQGAGQGAAAAAASGAGQGGYEGPGAG |
| | | QGAGAAAAAAGGAGQGGYGGLGGQGAGQGAGAAAAAAGGAGQGGYGGLGGQGAGQGAGA |
| | | AAAAAGGAGQGGYGGQGAGQGAAAAAAGGAGQGGYGGLGSGQGGYGRQGAGAAAAAAAA |
| | | GGAGQGGYGGLGGQGAGAAAAAAGGAGQGGYGGQGAGQGAAAAAASGAGQGGYEGPGAG |
| | | QGAGAAAAAAGGAGQGGYGGLGGQGAGQGAGAAAAAAGGAGQGGYGGLGGQGAGQGAGA |
| | | AAAAAGGAGQGGYGGQGAGQGAAAAAAGGAGQGGYGGLGSGQGGYGRQGAGAAAAAAAA |
| | | GGAGQGGYGGLGGQGAGAAAAAAGGAGQGGYGGQGAGQGAAAAAASGAGQGGYEGPGAG |
| | | QGAGAAAAAAGGAGQGGYGGLGGQGAGQGAGAAAAAAGGAGQGGYGGLGGQGAGQGAGA |
| | | AAAAAGGAGQGGYGGQGAGQGAAAAAAGGAGQGGYGGLGSGQGGYGRQGAGAAAAAAAA |
| 43 | NCMiSp 81 | GGYGSGASARAAAGAGGNSEQGGYGAGAGAAATAGSGAGGAGSYRRGSGAEATAGAGAG |
| | k | SAGGYGGQGGYGAGAGADAGSAGDYGRGAGAGAGAEAGTSSAGGYGEQGGYGTGAAAAA |
| | | GAGAGRAGGYGRGSGGAGGYGRPGAAGADGAGGYGGQGGYDAGAGAGAGGYGSGASARA |
| | | AAGAGGNSEQGGYGAGAGAAATAGSGAGGAGSYRRGSGAEATAGAGAGSAGGYGGQGGY |
| | | GAGAGADAGSAGDYGRGAGAGAGAEAGTSSAGGYGEQGGYGTGAAAAAGAGAGRAGGYG |
| | | RGSGGAGGYGRPGAAGADGAGGYGGQGGYDAGAGAGAGGYGSGASARAAAGAGGNSEQG |
| | | GYGAGAGAAATAGSGAGGAGSYRRGSGAEATAGAGAGSAGGYGGQGGYGAGAGADAGSA |
| | | GDYGRGAGAGAGAEAGTSSAGGYGEQGGYGTGAAAAAGAGAGRAGGYGRGSGGAGGYGR |
| | | PGAAGADGAGGYGGQGGYDAGAGAGAGGYGSGASARAAAGAGGNSEQGGYGAGAGAAAT |
| | | AGSGAGGAGSYRRGSGAEATAGAGAGSAGGYGGQGGYGAGAGADAGSAGDYGRGAGAGA |
| | | GAEAGTSSAGGYGEQGGYGTGAAAAAGAGAGRAGGYGRGSGGAGGYGRPGAAGADGAGG |
| | | YGGQGGYDAGAGAGAGGYGSGASARAAAGAGGNSEQGGYGAGAGAAATAGSGAGGAGSY |
| | | RRGSGAEATAGAGAGSAGGYGGQGGYGAGAGADAGSAGDYGRGAGAGAGAEAGTSSAGG |
| | | YGEQGGYGTGAAAAAGAGAGRAGGYGRGSGGAGGYGRPGAAGADGAGGYGGQGGYDAGA |
| | | GAGAGGYGSGASARAAAGAGGNSEQGGYGAGAGAAATAGSGAGGAGSYRRGSGAEATAG |
| | | AGAGSAGGYGGQGGYGAGAGADAGSAGDYGRGAGAGAGAEAGTSSAGGYGEQGGYGTGA |
| | | AAAAGAGAGRAGGYGRGSGGAGGYGRPGAAGADGAGGYGGQGGYDAGAGAGA |
| 44 | LTMiSp_v2 | GGYGQGSGGYGQNAGAAAGSGANGQGAGGYGQGAAAVAAAGAGAGGYGQGAGGYGQDAG |
| | 94k | GYGQGAGGNGQGVVDAAGYGPGSQGYGQSAAATSSAAAGASATGYTERQNEVVTTVTTT |
| | | RQETADRRQAARASAAVSTSAAAGYGQGTRGYGQVPGAAAGAGGYGQGAGGYGQGAAVG |
| | | SSAGSGVAGYGQGSGGYGQSAAAAAGAGAYGQGAGGYGQGAGAATGSGAGGCGQGAGGY |
| | | GQDAGAAAGAYGQGAGGYGQGAASGVATGTGAGGYGQGAGGYGQGASATAVAAAGAGAG |
| | | IIGQGAGVYGQSAVSAAGAAGDTGAGGYGQSTGGYGPGSGAGAGAAAGAGGYGPGSQGY |
| | | GQGAASTSSAAAGAGGYGQGSGGYGQNAGAAAGSGANGQGAGGYGQGAAAVAAAGAGAG |
| | | GYGQGAGGYGQDAGGYGQGAGGNGQGVVDAAGYGPGSQGYGQSAAATSSAAAGASATGY |
| | | TERQNEVVTTVTTTRQETADRRQAARASAAVSTSAAAGYGQGTRGYGQVPGAAAGAGGY |
| | | GQGAGGYGQGAAVGSSAGSGVAGYGQGSGGYGQSAAAAAGAGAYGQGAGGYGQGAGAAT |
| | | GSGAGGCGQGAGGYGQDAGAAAGAYGQGAGGYGQGAASGVATGTGAGGYGQGAGGYGQG |
| | | ASATAVAAAGAGAGIIGQGAGVYGQSAVSAAGAAGDTGAGGYGQSTGGYGPGSGAGAGA |
| | | AAGAGGYGPGSQGYGQGAASTSSAAAGAGGYGQGSGGYGQNAGAAAGSGANGQGAGGYG |
| | | QGAAAVAAAGAGAGGYGQGAGGYGQDAGGYGQGAGGNGQGVVDAAGYGPGSQGYGQSAA |
| | | ATSSAAAGASATGYTERQNEVVTTVTTTRQETADRRQAARASAAVSTSAAAGYGQGTRG |
| | | YGQVPGAAAGAGGYGQGAGGYGQGAAVGSSAGSGVAGYGQGSGGYGQSAAAAAGAGAYG |
| | | QGAGGYGQGAGAATGSGAGGCGQGAGGYGQDAGAAAGAYGQGAGGYGQGAASGVATGTG |
| | | AGGYGQGAGGYGQGASATAVAAAGAGAGIIGQGAGVYGQSAVSAAGAAGDTGAGGYGQS |
| | | TGGYGPGSGAGAGAAAGAGGYGPGSQGYGQGAASTSSAAAGA |
| 45 | LTMiSp_v2 | GGYGQGSGGYGQNAGAAAGSGANGQGAGGYGQGAAAVAAAGAGAGGYGQGAGGYGQDAG |
| | 94k | GYGQGAGGNGQGVVDAAGYGPGSQGYGQSAAATSSAAAGASATGYTERQNEVVTTVTTT |
| | | RQETADRRQAARASAAVSTSAAAGYGQGTRGYGQVPGAAAGAGGYGQGAGGYGQGAAVG |
| | | SSAGSGVAGYGQGSGGYGQSAAAAAGAGAYGQGAGGYGQGAGAATGSGAGGCGQGAGGY |
| | | GQDAGAAAGAYGQGAGGYGQGAASGVATGTGAGGYGQGAGGYGQGASATAVAAAGAGAG |
| | | IIGQGAGVYGQSAVSAAGAAGDTGAGGYGQSTGGYGPGSGAGAGAAAGAGGYGPGSQGY |
| | | GQGAASTSSAAAGAGGYGQGSGGYGQNAGAAAGSGANGQGAGGYGQGAAAVAAAGAGAG |
| | | GYGQGAGGYGQDAGGYGQGAGGNGQGVVDAAGYGPGSQGYGQSAAATSSAAAGASATGY |
| | | TERQNEVVTTVTTTRQETADRRQAARASAAVSTSAAAGYGQGTRGYGQVPGAAAGAGGY |
| | | GQGAGGYGQGAAVGSSAGSGVAGYGQGSGGYGQSAAAAAGAGAYGQGAGGYGQGAGAAT |
| | | GSGAGGCGQGAGGYGQDAGAAAGAYGQGAGGYGQGAASGVATGTGAGGYGQGAGGYGQG |
| | | ASATAVAAAGAGAGIIGQGAGVYGQSAVSAAGAAGDTGAGGYGQSTGGYGPGSGAGAGA |
| | | AAGAGGYGPGSQGYGQGAASTSSAAAGAGGYGQGSGGYGQNAGAAAGSGANGQGAGGYG |
| | | QGAAAVAAAGAGAGGYGQGAGGYGQDAGGYGQGAGGNGQGVVDAAGYGPGSQGYGQSAA |
| | | ATSSAAAGASATGYTERQNEVVTTVTTTRQETADRRQAARASAAVSTSAAAGYGQGTRG |
| | | YGQVPGAAAGAGGYGQGAGGYGQGAAVGSSAGSGVAGYGQGSGGYGQSAAAAAGAGAYG |
| | | QGAGGYGQGAGAATGSGAGGCGQGAGGYGQDAGAAAGAYGQGAGGYGQGAASGVATGTG |
| | | AGGYGQGAGGYGQGASATAVAAAGAGAGIIGQGAGVYGQSAVSAAGAAGDTGAGGYGQS |
| | | TGGYGPGSGAGAGAAAGAGGYGPGSQGYGQGAASTSSAAAGA |
| 46 | LGMiSp_v1 | GGYTQKQNEVITTVSTTRQKIADYGQKQASGASAAVSTSSAGGYAQGPGGYGPGKGAGA |
| | 80k | TTGAGARGYSQGPGGYAQGVSTAAGAAIAGAGGYGPSTGPYGQGAIDASGGYGQGVGTA |
| | | AGASASAGSGAATGVGPVGYGQGLGGYGQAVGQGAGGYGQGAGAATVTVTAAVPGGYGP |
| | | GAGGYGQGVGAAAGAGTDAGIGGYGQGAGGFGQGGAAASAATGAGPGGYGLGAGGYGQP |
| | | IGATAGATAGAGGYGQGAGVSGAGSRGAPAGYGPGAGPAAGATSGAVAGGKGPGGYGPS |
| | | QVASASSSAAAAAASGGYTQKQNEVITTVSTTRQKIADYGQKQASGASAAVSTSSAGGY |
| | | AQGPGGYGPGKGAGATTGAGARGYSQGPGGYAQGVSTAAGAAIAGAGGYGPSTGPYGQG |
| | | AIDASGGYGQGVGTAAGASASAGSGAATGVGPVGYGQGLGGYGQAVGQGAGGYGQGAGA |
| | | ATVTVTAAVPGGYGPGAGGYGQGVGAAAGAGTDAGIGGYGQGAGGFGQGGAAASAATGA |
| | | GPGGYGLGAGGYGQPIGATAGATAGAGGYGQGAGVSGAGSRGAPAGYGPGAGPAAGATS |
| | | GAVAGGKGPGGYGPSQVASASSSAAAAAASGGYTQKQNEVITTVSTTRQKIADYGQKQA |
| | | SGASAAVSTSSAGGYAQGPGGYGPGKGAGATTGAGARGYSQGPGGYAQGVSTAAGAAIA |
| | | GAGGYGPSTGPYGQGAIDASGGYGQGVGTAAGASASAGSGAATGVGPVGYGQGLGGYGQ |
| | | AVGQGAGGYGQGAGAATVTVTAAVPGGYGPGAGGYGQGVGAAAGAGTDAGIGGYGQGAG |
| | | GFGQGGAAASAATGAGPGGYGLGAGGYGQPIGATAGATAGAGGYGQGAGVSGAGSRGAP |
| | | AGYGPGAGPAAGATSGAVAGGKGPGGYGPSQVASASSSAAAAAAS |
| 47 | LGMiSp_v1 | GGYTQKQNEVITTVSTTRQKIADYGQKQASGASAAVSTSSAGGYAQGPGGYGPGKGAGA |
| | 80k | TTGAGARGYSQGPGGYAQGVSTAAGAAIAGAGGYGPSTGPYGQGAIDASGGYGQGVGTA |
| | | AGASASAGSGAATGVGPVGYGQGLGGYGQAVGQGAGGYGQGAGAATVTVTAAVPGGYGP |
| | | GAGGYGQGVGAAAGAGTDAGIGGYGQGAGGFGQGGAAASAATGAGPGGYGLGAGGYGQP |
| | | IGATAGATAGAGGYGQGAGVSGAGSRGAPAGYGPGAGPAAGATSGAVAGGKGPGGYGPS |
| | | QVASASSSAAAAAASGGYTQKQNEVITTVSTTRQKIADYGQKQASGASAAVSTSSAGGY |
| | | AQGPGGYGPGKGAGATTGAGARGYSQGPGGYAQGVSTAAGAAIAGAGGYGPSTGPYGQG |
| | | AIDASGGYGQGVGTAAGASASAGSGAATGVGPVGYGQGLGGYGQAVGQGAGGYGQGAGA |
| | | ATVTVTAAVPGGYGPGAGGYGQGVGAAAGAGTDAGIGGYGQGAGGFGQGGAAASAATGA |
| | | GPGGYGLGAGGYGQPIGATAGATAGAGGYGQGAGVSGAGSRGAPAGYGPGAGPAAGATS |
| | | GAVAGGKGPGGYGPSQVASASSSAAAAAASGGYTQKQNEVITTVSTTRQKIADYGQKQA |
| | | SGASAAVSTSSAGGYAQGPGGYGPGKGAGATTGAGARGYSQGPGGYAQGVSTAAGAAIA |
| | | GAGGYGPSTGPYGQGAIDASGGYGQGVGTAAGASASAGSGAATGVGPVGYGQGLGGYGQ |
| | | AVGQGAGGYGQGAGAATVTVTAAVPGGYGPGAGGYGQGVGAAAGAGTDAGIGGYGQGAG |
| | | GFGQGGAAASAATGAGPGGYGLGAGGYGQPIGATAGATAGAGGYGQGAGVSGAGSRGAP |
| | | AGYGPGAGPAAGATSGAVAGGKGPGGYGPSQVASASSSAAAAAAS |
| 48 | LH MiSp 58 | GGYGRGQGAGAGVGAGAGAAAGAAAIARAGGYGQGAGGYGQGQGAGAAAGAAAGAGAGG |
| | k2 | YGQGAGGYGRGQGAGAGAGAGAGARGYGQGAGAGAAAGAAASAGAGGYGQGAGGYGQGQ |
| | | GAGAAAGAAASAGAGGYGQGAGGYGQGQGAGGYGRGQGAGAGVGAGAGAAAGAAAIARA |
| | | GGYGQGAGGYGQGQGAGAAAGAAAGAGAGGYGQGAGGYGRGQGAGAGAGAGAGARGYGQ |
| | | GAGAGAAAGAAASAGAGGYGQGAGGYGQGQGAGAAAGAAASAGAGGYGQGAGGYGQGQG |
| | | AGGYGRGQGAGAGVGAGAGAAAGAAAIARAGGYGQGAGGYGQGQGAGAAAGAAAGAGAG |
| | | GYGQGAGGYGRGQGAGAGAGAGAGARGYGQGAGAGAAAGAAASAGAGGYGQGAGGYGQG |
| | | QGAGAAAGAAASAGAGGYGQGAGGYGQGQGAGGYGRGQGAGAGVGAGAGAAAGAAAIAR |
| | | AGGYGQGAGGYGQGQGAGAAAGAAAGAGAGGYGQGAGGYGRGQGAGAGAGAGAGARGYG |
| | | QGAGAGAAAGAAASAGAGGYGQGAGGYGQGQGAGAAAGAAASAGAGGYGQGAGGYGQGQ |
| | | GAGGYGRGQGAGAGVGAGAGAAAGAAAIARAGGYGQGAGGYGQGQGAGAAAGAAAGAGA |
| | | GGYGQGAGGYGRGQGAGAGAGAGAGARGYGQGAGAGAAAGAAASAGAGGYGQGAGGYGQ |
| | | GQGAGAAAGAAASAGAGGYGQGAGGYGQGQGA |
| 49 | LH MiSp_v1 | GGYGQGAGGYGQGAGAAAGAAAGAGAGGYGRGAGSAAGAAAGAGVGEYGQGAGGYGQGA |
| | 80k | GAAAGAAAGAGAGGYGQGAGGYGQGAGGYGQGAGAAAGAGAGSYGQGAGGYGQGAGAAA |
| | | GAAAGAGAGGYGQGAGGYGQGAGAAAGAAAGAGAGGYGQGAGGYGQGAGAAAGAGAGGY |
| | | GRGAGSAAGAAAGSGAGGYGQGAGGYGQGAGAGAGGYGQGAGASTGAAAGAGAGGYGQG |
| | | AGGYGQGSGAAAGAGGYGQGSQGYEQGAAATSSAAAGASSTGYTERQNEVVTTVTTTRQ |
| | | EIADRRQAASASGAVSTSAAAGYGQGAGTGGYGQGAGGYGQGAGAAAGAAAGAGAGGYG |
| | | RGAGSAAGAAAGAGVGEYGQGAGGYGQGAGAAAGAAAGAGAGGYGQGAGGYGQGAGGYG |
| | | QGAGAAAGAGAGSYGQGAGGYGQGAGAAAGAAAGAGAGGYGQGAGGYGQGAGAAAGAAA |
| | | GAGAGGYGQGAGGYGQGAGAAAGAGAGGYGRGAGSAAGAAAGSGAGGYGQGAGGYGQGA |
| | | GAGAGGYGQGAGASTGAAAGAGAGGYGQGAGGYGQGSGAAAGAGGYGQGSQGYEQGAAA |
| | | TSSAAAGASSTGYTERQNEVVTTVTTTRQEIADRRQAASASGAVSTSAAAGYGQGAGTG |
| | | GYGQGAGGYGQGAGAAAGAAAGAGAGGYGRGAGSAAGAAAGAGVGEYGQGAGGYGQGAG |
| | | AAAGAAAGAGAGGYGQGAGGYGQGAGGYGQGAGAAAGAGAGSYGQGAGGYGQGAGAAAG |
| | | AAAGAGAGGYGQGAGGYGQGAGAAAGAAAGAGAGGYGQGAGGYGQGAGAAAGAGAGGYG |
| | | RGAGSAAGAAAGSGAGGYGQGAGGYGQGAGAGAGGYGQGAGASTGAAAGAGAGGYGQGA |
| | | GGYGQGSGAAAGAGGYGQGSQGYEQGAAATSSAAAGASSTGYTERQNEVVTTVTTTRQE |
| | | IADRRQAASASGAVSTSAAAGYGQGAGT |
| 50 | LH MiSp_v1 | GGYGQGAGGYGQGAGAAAGAAAGAGAGGYGRGAGSAAGAAAGAGVGEYGQGAGGYGQGA |
| | 80k | GAAAGAAAGAGAGGYGQGAGGYGQGAGGYGQGAGAAAGAGAGSYGQGAGGYGQGAGAAA |
| | | GAAAGAGAGGYGQGAGGYGQGAGAAAGAAAGAGAGGYGQGAGGYGQGAGAAAGAGAGGY |
| | | GRGAGSAAGAAAGSGAGGYGQGAGGYGQGAGAGAGGYGQGAGASTGAAAGAGAGGYGQG |
| | | AGGYGQGSGAAAGAGGYGQGSQGYEQGAAATSSAAAGASSTGYTERQNEVVTTVTTTRQ |
| | | EIADRRQAASASGAVSTSAAAGYGQGAGTGGYGQGAGGYGQGAGAAAGAAAGAGAGGYG |
| | | RGAGSAAGAAAGAGVGEYGQGAGGYGQGAGAAAGAAAGAGAGGYGQGAGGYGQGAGGYG |
| | | QGAGAAAGAGAGSYGQGAGGYGQGAGAAAGAAAGAGAGGYGQGAGGYGQGAGAAAGAAA |
| | | GAGAGGYGQGAGGYGQGAGAAAGAGAGGYGRGAGSAAGAAAGSGAGGYGQGAGGYGQGA |
| | | GAGAGGYGQGAGASTGAAAGAGAGGYGQGAGGYGQGSGAAAGAGGYGQGSQGYEQGAAA |
| | | TSSAAAGASSTGYTERQNEVVTTVTTTRQEIADRRQAASASGAVSTSAAAGYGQGAGTG |
| | | GYGQGAGGYGQGAGAAAGAAAGAGAGGYGRGAGSAAGAAAGAGVGEYGQGAGGYGQGAG |
| | | AAAGAAAGAGAGGYGQGAGGYGQGAGGYGQGAGAAAGAGAGSYGQGAGGYGQGAGAAAG |
| | | AAAGAGAGGYGQGAGGYGQGAGAAAGAAAGAGAGGYGQGAGGYGQGAGAAAGAGAGGYG |
| | | RGAGSAAGAAAGSGAGGYGQGAGGYGQGAGAGAGGYGQGAGASTGAAAGAGAGGYGQGA |
| | | GGYGQGSGAAAGAGGYGQGSQGYEQGAAATSSAAAGASSTGYTERQNEVVTTVTTTRQE |
| | | IADRRQAASASGAVSTSAAAGYGQGAGT |
| 51 | NCr MiSp | GAGAGVGGAGGYGSGAGAGAGAGAGAASGAAAGAAAGAGAGGAGGYGTGQGYGAGAGAG |
| | 67k1 | AGAGAGGAGGYGRGAGAGAGAGAGGAGGYGAGQGYGAGAGAGAAAAAGDGAGAGGAGGY |
| | | GRGAGAGAGAGAAAGAGAGGAGGYGAGQGYGAGAGAGAAAGAGAGGAGGYGAGQGYGAG |
| | | AGAGAAAAAGAGAGVGGAGGYGSGAGAGAGAGAGAASGAAAGAAAGAGAGGAGGYGTGQ |
| | | GYGAGAGAGAGAGAGGAGGYGRGAGAGAGAGAGGAGGYGAGQGYGAGAGAGAAAAAGDG |
| | | AGAGGAGGYGRGAGAGAGAGAAAGAGAGGAGGYGAGQGYGAGAGAGAAAGAGAGGAGGY |
| | | GAGQGYGAGAGAGAAAAAGAGAGVGGAGGYGSGAGAGAGAGAGAASGAAAGAAAGAGAG |
| | | GAGGYGTGQGYGAGAGAGAGAGAGGAGGYGRGAGAGAGAGAGGAGGYGAGQGYGAGAGA |
| | | GAAAAAGDGAGAGGAGGYGRGAGAGAGAGAAAGAGAGGAGGYGAGQGYGAGAGAGAAAG |
| | | AGAGGAGGYGAGQGYGAGAGAGAAAAAGAGAGVGGAGGYGSGAGAGAGAGAGAASGAAA |
| | | GAAAGAGAGGAGGYGTGQGYGAGAGAGAGAGAGGAGGYGRGAGAGAGAGAGGAGGYGAG |
| | | QGYGAGAGAGAAAAAGDGAGAGGAGGYGRGAGAGAGAGAAAGAGAGGAGGYGAGQGYGA |
| | | GAGAGAAAGAGAGGAGGYGAGQGYGAGAGAGAAAAAGAGAGVGGAGGYGSGAGAGAGAG |
| | | AGAASGAAAGAAAGAGAGGAGGYGTGQGYGAGAGAGAGAGAGGAGGYGRGAGAGAGAGA |
| | | GGAGGYGAGQGYGAGAGAGAAAAAGDGAGAGGAGGYGRGAGAGAGAGAAAGAGAGGAGG |
| | | YGAGQGYGAGAGAGAAAGAGAGGAGGYGAGQGYGAGAGAGAAAAA |
| 52 | LTMiSp_v1 | GGYGQGAGAGAGAGAGAGAGAAAGAGAGGYGQGAGGYGRGQGAAAAAGAGAGGYGQGAG |
| | 71k | AGAGAGAGAAAGAGAGGYGQGAGGYGKGQGAAAAAFAGAGGYGQGAGAGAGAYAGAGAG |
| | | AVAGAAAGAGAGGYGQGAGGYGRGQGAAAAGAGAGAGGYGQGAGAGAGAAANAGAGGYG |
| | | QGAGGYGRGQGAAAAAGAGAGAGGYGQGAGAGTGAAAGAGASAGAGVGAGAGAAAGAAA |
| | | GAGAGGYGQGAGGYGPGQGAAAAAGAGAGAGGYGQGSGAGAGAGAGAAAGAGAGGYGQG |
| | | AGGYGRGQGAAAAGAGGYGQGAGAGAGAGAGAGAGAAAGAGAGGYGQGAGGYGRGQGAA |
| | | AAAGAGAGGYGQGAGAGAGAGAGAAAGAGAGGYGQGAGGYGKGQGAAAAAFAGAGGYGQ |
| | | GAGAGAGAYAGAGAGAVAGAAAGAGAGGYGQGAGGYGRGQGAAAAGAGAGAGGYGQGAG |
| | | AGAGAAANAGAGGYGQGAGGYGRGQGAAAAAGAGAGAGGYGQGAGAGTGAAAGAGASAG |
| | | AGVGAGAGAAAGAAAGAGAGGYGQGAGGYGPGQGAAAAAGAGAGAGGYGQGSGAGAGAG |
| | | AGAAAGAGAGGYGQGAGGYGRGQGAAAAGAGGYGQGAGAGAGAGAGAGAGAAAGAGAGG |
| | | YGQGAGGYGRGQGAAAAAGAGAGGYGQGAGAGAGAGAGAAAGAGAGGYGQGAGGYGKGQ |
| | | GAAAAAFAGAGGYGQGAGAGAGAYAGAGAGAVAGAAAGAGAGGYGQGAGGYGRGQGAAA |
| | | AGAGAGAGGYGQGAGAGAGAAANAGAGGYGQGAGGYGRGQGAAAAAGAGAGAGGYGQGA |
| | | GAGTGAAAGAGASAGAGVGAGAGAAAGAAAGAGAGGYGQGAGGYGPGQGAAAAAGAGAG |
| | | AGGYGQGSGAGAGAGAGAAAGAGAGGYGQGAGGYGRGQGAAAAGA |
| 53 | UDMi Sp | GAGAGYRGQAGYIQGAGASAGAAAAGAGVGYGGQAGYGQGAGASAGAAAAAGAGAGRQA |
| | 32k | GYGQGAGASAGAAAAGAGAGRQAGYGQGAGASAGAAAAGADAGYGGQAGYGQGAGASAG |
| | | AAASGAGAGYGGQAGYGQGAGASAGAAAAGAGAGYLGQAGYGQGAGASAGAAAGAGAGY |
| | | GGQAGYGQGTGAAASAAASSAGAGAGYRGQAGYIQGAGASAGAAAAGAGVGYGGQAGYG |
| | | QGAGASAGAAAAAGAGAGRQAGYGQGAGASAGAAAAGAGAGRQAGYGQGAGASAGAAAA |
| | | GADAGYGGQAGYGQGAGASAGAAASGAGAGYGGQAGYGQGAGASAGAAAAGAGAGYLGQ |
| | | AGYGQGAGASAGAAAGAGAGYGGQAGYGQGTGAAASAAASSA |
| 54 | UDMi Sp | GAGAGYRGQAGYIQGAGASAGAAAAGAGVGYGGQAGYGQGAGASAGAAAAAGAGAGRQA |
| | 64k | GYGQGAGASAGAAAAGAGAGRQAGYGQGAGASAGAAAAGADAGYGGQAGYGQGAGASAG |
| | | AAASGAGAGYGGQAGYGQGAGASAGAAAAGAGAGYLGQAGYGQGAGASAGAAAGAGAGY |
| | | GGQAGYGQGTGAAASAAASSAGAGAGYRGQAGYIQGAGASAGAAAAGAGVGYGGQAGYG |
| | | QGAGASAGAAAAAGAGAGRQAGYGQGAGASAGAAAAGAGAGRQAGYGQGAGASAGAAAA |
| | | GADAGYGGQAGYGQGAGASAGAAASGAGAGYGGQAGYGQGAGASAGAAAAGAGAGYLGQ |
| | | AGYGQGAGASAGAAAGAGAGYGGQAGYGQGTGAAASAAASSAGAGAGYRGQAGYIQGAG |
| | | ASAGAAAAGAGVGYGGQAGYGQGAGASAGAAAAAGAGAGRQAGYGQGAGASAGAAAAGA |
| | | GAGRQAGYGQGAGASAGAAAAGADAGYGGQAGYGQGAGASAGAAASGAGAGYGGQAGYG |
| | | QGAGASAGAAAAGAGAGYLGQAGYGQGAGASAGAAAGAGAGYGGQAGYGQGTGAAASAA |
| | | ASSAGAGAGYRGQAGYIQGAGASAGAAAAGAGVGYGGQAGYGQGAGASAGAAAAAGAGA |
| | | GRQAGYGQGAGASAGAAAAGAGAGRQAGYGQGAGASAGAAAAGADAGYGGQAGYGQGAG |
| | | ASAGAAASGAGAGYGGQAGYGQGAGASAGAAAAGAGAGYLGQAGYGQGAGASAGAAAGA |
| | | GAGYGGQAGYGQGTGAAASAAASSA |
| 55 | UDMi Sp | GAGAGYRGQAGYIQGAGASAGAAAAGAGVGYGGQAGYGQGAGASAGAAAAAGAGAGRQA |
| | 127k | GYGQGAGASAGAAAAGAGAGRQAGYGQGAGASAGAAAAGADAGYGGQAGYGQGAGASAG |
| | | AAASGAGAGYGGQAGYGQGAGASAGAAAAGAGAGYLGQAGYGQGAGASAGAAAGAGAGY |
| | | GGQAGYGQGTGAAASAAASSAGAGAGYRGQAGYIQGAGASAGAAAAGAGVGYGGQAGYG |
| | | QGAGASAGAAAAAGAGAGRQAGYGQGAGASAGAAAAGAGAGRQAGYGQGAGASAGAAAA |
| | | GADAGYGGQAGYGQGAGASAGAAASGAGAGYGGQAGYGQGAGASAGAAAAGAGAGYLGQ |
| | | AGYGQGAGASAGAAAGAGAGYGGQAGYGQGTGAAASAAASSAGAGAGYRGQAGYIQGAG |
| | | ASAGAAAAGAGVGYGGQAGYGQGAGASAGAAAAAGAGAGRQAGYGQGAGASAGAAAAGA |
| | | GAGRQAGYGQGAGASAGAAAAGADAGYGGQAGYGQGAGASAGAAASGAGAGYGGQAGYG |
| | | QGAGASAGAAAAGAGAGYLGQAGYGQGAGASAGAAAGAGAGYGGQAGYGQGTGAAASAA |
| | | ASSAGAGAGYRGQAGYIQGAGASAGAAAAGAGVGYGGQAGYGQGAGASAGAAAAAGAGA |
| | | GRQAGYGQGAGASAGAAAAGAGAGRQAGYGQGAGASAGAAAAGADAGYGGQAGYGQGAG |
| | | ASAGAAASGAGAGYGGQAGYGQGAGASAGAAAAGAGAGYLGQAGYGQGAGASAGAAAGA |
| | | GAGYGGQAGYGQGTGAAASAAASSAGAGAGYRGQAGYIQGAGASAGAAAAGAGVGYGGQ |
| | | AGYGQGAGASAGAAAAAGAGAGRQAGYGQGAGASAGAAAAGAGAGRQAGYGQGAGASAG |
| | | AAAAGADAGYGGQAGYGQGAGASAGAAASGAGAGYGGQAGYGQGAGASAGAAAAGAGAG |
| | | YLGQAGYGQGAGASAGAAAGAGAGYGGQAGYGQGTGAAASAAASSAGAGAGYRGQAGYI |
| | | QGAGASAGAAAAGAGVGYGGQAGYGQGAGASAGAAAAAGAGAGRQAGYGQGAGASAGAA |
| | | AAGAGAGRQAGYGQGAGASAGAAAAGADAGYGGQAGYGQGAGASAGAAASGAGAGYGGQ |
| | | AGYGQGAGASAGAAAAGAGAGYLGQAGYGQGAGASAGAAAGAGAGYGGQAGYGQGTGAA |
| | | ASAAASSAGAGAGYRGQAGYIQGAGASAGAAAAGAGVGYGGQAGYGQGAGASAGAAAAA |
| | | GAGAGRQAGYGQGAGASAGAAAAGAGAGRQAGYGQGAGASAGAAAAGADAGYGGQAGYG |
| | | QGAGASAGAAASGAGAGYGGQAGYGQGAGASAGAAAAGAGAGYLGQAGYGQGAGASAGA |
| | | AAGAGAGYGGQAGYGQGTGAAASAAASSAGAGAGYRGQAGYIQGAGASAGAAAAGAGVG |
| | | YGGQAGYGQGAGASAGAAAAAGAGAGRQAGYGQGAGASAGAAAAGAGAGRQAGYGQGAG |
| | | ASAGAAAAGADAGYGGQAGYGQGAGASAGAAASGAGAGYGGQAGYGQGAGASAGAAAAG |
| | | AGAGYLGQAGYGQGAGASAGAAAGAGAGYGGQAGYGQGTGAAASAAASSA |
| 56 | NCMi Sp | GAGAGGAGYGRGAGAGAGAAAGAGAGAAAGAGAGAGGYGGQGGYGAGAGAGAAAAAGAG |
| | 35k1 | AGGAAGYSRGGRAGAAGAGAGAAAGAGAGAGGYGGQGGYGAGAGAGAAAAAGAGSGGAG |
| | | GYGRGAGAGAAAGAGAAAGAGAGAGGYGGQGGYGAGAGAAAAAGAGAGGAGYGRGAGAG |
| | | AGAAAGAGAGAAAGAGAGAGGYGGQGGYGAGAGAGAAAAAGAGAGGAAGYSRGGRAGAA |
| | | GAGAGAAAGAGAGAGGYGGQGGYGAGAGAGAAAAAGAGSGGAGGYGRGAGAGAAAGAGA |
| | | AAGAGAGAGGYGGQGGYGAGAGAAAAAGAGAGGAGYGRGAGAGAGAAAGAGAGAAAGAG |
| | | AGAGGYGGQGGYGAGAGAGAAAAAGAGAGGAAGYSRGGRAGAAGAGAGAAAGAGAGAGG |
| | | YGGQGGYGAGAGAGAAAAAGAGSGGAGGYGRGAGAGAAAGAGAAAGAGAGAGGYGGQGG |
| | | YGAGAGAAAA |
| 57 | NCMiSp | GGYGAVAGGSGAGASAGVGAGAGSVAGYGGQGGYGAGTGAGAGSAGGYGRGTGAGTAAG |
| | 63k | SGAGAAAGAGAGAAAGAGAGAAAGAGAGAGSLGGYEGQGAYSAGVGAGAAAAAGAGAGS |
| | | VGGYGRGAGVGAGAAAGSAAGAGGAGGYRRDGGYGAGAGAGATAAASSGAGSAGGYGRG |
| | | AGAGAAAVAGADAGGYGRNSGAGTAAYAGARAGSAGVYGGQGGYSSGAGASAASGAGAD |
| | | ITSGYGRGDGAGAGAGTIGAGGYGGGAGSGAAAAGGYGAVAGGSGAGASAGVGAGAGSV |
| | | AGYGGQGGYGAGTGAGAGSAGGYGRGTGAGTAAGSGAGAAAGAGAGAAAGAGAGAAAGA |
| | | GAGAGSLGGYEGQGAYSAGVGAGAAAAAGAGAGSVGGYGRGAGVGAGAAAGSAAGAGGA |
| | | GGYRRDGGYGAGAGAGATAAASSGAGSAGGYGRGAGAGAAAVAGADAGGYGRNSGAGTA |
| | | AYAGARAGSAGVYGGQGGYSSGAGASAASGAGADITSGYGRGDGAGAGAGTIGAGGYGG |
| | | GAGSGAAAAGGYGAVAGGSGAGASAGVGAGAGSVAGYGGQGGYGAGTGAGAGSAGGYGR |
| | | GTGAGTAAGSGAGAAAGAGAGAAAGAGAGAAAGAGAGAGSLGGYEGQGAYSAGVGAGAA |
| | | AAAGAGAGSVGGYGRGAGVGAGAAAGSAAGAGGAGGYRRDGGYGAGAGAGATAAASSGA |
| | | GSAGGYGRGAGAGAAAVAGADAGGYGRNSGAGTAAYAGARAGSAGVYGGQGGYSSGAGA |
| | | SAASGAGADITSGYGRGDGAGAGAGTIGAGGYGGGAGSGAAAA |
| 58 | UDMi Sp | GSGAGAGSGYGAGAGAGAGSGYGAGSSASAGSAINTQTVTSSTTTSSQSSAAATGAGYG |
| | 63k | TGAGTGASAGAAASGAGAGYGGQAGYGQGAGASARAAGSGYGAGAGAAAAAGSGYGAGA |
| | | GAGAGSGYGAGAAAGSGAGAGSGYGAGAGAGAGSGYGAGSSASAGSAINTQTVTSSTTT |
| | | SSQSSAAATGAGYGTGAGTGASAGAAASGAGAGYGGQAGYGQGAGASARAAGSGYGAGA |
| | | GAAAAAGSGYGAGAGAGAGSGYGAGAAAGSGAGAGSGYGAGAGAGAGSGYGAGSSASAG |
| | | SAINTQTVTSSTTTSSQSSAAATGAGYGTGAGTGASAGAAASGAGAGYGGQAGYGQGAG |
| | | ASARAAGSGYGAGAGAAAAAGSGYGAGAGAGAGSGYGAGAAGSGAGAGSGYGAGAGAGA |
| | | GSGYGAGSSASAGSAINTQTVTSSTTTSSQSSAAATGAGYGTGAGTGASAGAAASGAGA |
| | | GYGGQAGYGQGAGASARAAGSGYGAGAGAAAAAGSGYGAGAGAGAGSGYGAGAAAGSGA |
| | | GAGSGYGAGAGAGAGSGYGAGSSASAGSAINTQTVTSSTTTSSQSSAAATGAGYGTGAG |
| | | TGASAGAAASGAGAGYGGQAGYGQGAGASARAAGSGYGAGAGAAAAAGSGYGAGAGAGA |
| | | GSGYGAGAAAGSGAGAGSGYGAGAGAGAGSGYGAGSSASAGSAINTQTVTSSTTTSSQS |
| | | SAAATGAGYGTGAGTGASAGAAASGAGAGYGGQAGYGQGAGASARAAGSGYGAGAGAAA |
| | | AAGSGYGAGAGAGAGSGYGAGAA |
| 59 | UDMi Sp | GSGAGAGSGYGAGAGAGAGSGYGAGSSASAGSAINTQTVTSSTTTSSQSSAAATGAGYG |
| | 31k2 | TGAGTGASAGAAASGAGAGYGGQAGYGQGAGASARAAGSGYGAGAGAAAAAGSGYGAGA |
| | | GAGAGSGYGAGAAAGSGAGAGSGYGAGAGAGAGSGYGAGSSASAGSAINTQTVTSSTTT |
| | | SSQSSAAATGAGYGTGAGTGASAGAAASGAGAGYGGQAGYGQGAGASARAAGSGYGAGA |
| | | GAAAAAGSGYGAGAGAGAGSGYGAGAAAGSGAGAGSGYGAGAGAGAGSGYGAGSSASAG |
| | | SAINTQTVTSSTTTSSQSSAAATGAGYGTGAGTGASAGAAASGAGAGYGGQAGYGQGAG |
| | | ASARAAGSGYGAGAGAAAAAGSGYGAGAGAGAGSGYGAGAA |
| 60 | His | GGGGGGHHHHHH |
| 61 | Flag | GDYKDDDDKDYKDDDDKDYKDDDDK |
| 62 | Flag-His | GDYKDDDDKDYKDDDDKDYKDDDDKGHHHHHH |

1. A method of solubilizing a recombinant spider silk protein from a host cell, comprising:
providing a cell culture comprising a host cell, wherein the host cell expresses a recombinant spider silk protein;
collecting an insoluble portion derived from the cell culture, wherein the insoluble portion comprises the recombinant spider silk protein; and
adding the insoluble portion of the host cell to a solution comprising a salt and an alcohol, thereby solubilizing the recombinant spider silk protein in the solution.

2. The method of claim 1, wherein the salt comprises a calcium salt.

3. The method of claim 2, wherein the calcium salt comprises at least one of calcium chloride, calcium nitrate, calcium thiocyanate, calcium iodide, or calcium bromide.

4. The method of claim 3, wherein the calcium salt comprises calcium chloride.

5. The method of any one of claims 1-4, wherein the solution comprises at least 1M, 1.5M, 2M, 2.5M, 3M, or 4M calcium chloride.

6. The method of claim 4, wherein the solution comprises at least 2M calcium chloride.

7. The method of claim 3, wherein the calcium salt comprises calcium nitrate.

8. The method of claim 1, wherein the salt comprises a strontium salt or a barium salt.

9. The method of any one of claims 1-8, wherein the insoluble portion is at least 5%, 10%, 15%, 20%, 25%, 30%, or 35% (w/v) of the solution volume.

10. The method of claim 9, wherein the insoluble portion is about 15% (w/v) of the solution volume.

11. The method of claim 9, wherein the insoluble portion is at most about 35% (w/v) of the solution volume.

12. The method of any one of claims 1-11, wherein the ratio of the volume of the solution to the insoluble portion is at least 3×, 5× or 7×.

13. The method of claim 12, wherein the ratio of the volume of the solution to the insoluble portion is at least 3×.

14. The method of claim 12, wherein the ratio of the volume of the solution to the insoluble portion is about 7×.

15. The method of any of the above claims, wherein the alcohol comprises at least one of methanol, ethanol, or isopropanol.

16. The method of claim 15, wherein the alcohol comprises methanol.

17. The method of any one of claims 1-16, wherein the solution comprises 2M calcium chloride and methanol.

18. The method of any one of claims 1-17, wherein the insoluble portion is incubated with the solution at a temperature between 20° C. and 70° C.

19. The method of claim 18, wherein the insoluble portion is incubated at room temperature.

20. The method of claim 18, wherein the insoluble portion is incubated at about 35° C.

21. The method of claim 18, wherein the insoluble portion is incubated at about 55° C.

22. The method of claim 18, wherein the insoluble portion is incubated at no more than 70° C.

23. The method of claim 18, wherein the insoluble portion is incubated at no less than 20° C.

24. The method of any one of claims 1-24, wherein the insoluble portion is incubated in the solution for 15 to 120 minutes.

25. The method of claim 24, wherein the insoluble portion is incubated in the solution for 30 min.

26. The method of any one of claims 1-25, further comprising evaporating the alcohol.

27. The method of any one of claims 1-26, wherein the insoluble portion comprises a cell lysate pellet.

28. The method of any one of claims 1-27, wherein collecting the insoluble portion derived from the cell culture comprises lysing the host cell.

29. The method of claim 28, wherein lysing comprises heat treatment, chemical treatment, shear disruption, physical homogenization, microfluidization, sonication, or chemical homogenization.

30. The method of claims 28 to 29, wherein collecting the insoluble portion of the cell culture further comprises centrifuging the lysed cell to obtain a cell lysate pellet.

31. The method of one of claims 1-30, further comprising removing impurities from the solution.

32. The method of claim 31, wherein removing impurities comprises adding an aqueous solution to precipitate the impurities.

33. The method of claim 32, wherein the aqueous solution is water.

34. The method of claim 31, wherein removing the impurities comprises filtration, centrifugation, gravitational settling, adsorption, dialysis, or phase separation.

35. The method of claim 34, wherein the filtration is ultrafiltration, microfiltration, or diafiltration.

36. The method of any one of claims 1-35, wherein the solubilized recombinant spider silk protein comprises at least 60%, at least 65%, at least 70%, at least 75%, at least 80%, at least 85%, at least 90%, or at least 95% full-length recombinant spider silk protein.

37. The method of any one of claims 1-36, further comprising isolating the recombinant spider silk protein from the solution, thereby producing an isolated recombinant spider silk protein.

38. The method of claim 37, wherein an amount of isolated recombinant spider silk protein is measured using a Western blot.

39. The method of claim 37 or 38, wherein an amount of isolated recombinant spider silk protein is measured using an ELISA.

40. The method of any of claims 37-39, wherein an amount of isolated recombinant spider silk protein is measured using Size Exclusion Chromatography.

41. The method of any of claims 37-40, wherein the isolated recombinant spider silk protein is a full-length recombinant spider silk protein.

42. The method of any of claims 37-40, wherein the isolated recombinant spider silk protein comprises at least 60%, at least 65%, at least 70%, at least 75%, at least 80%, at least 85%, at least 90%, or at least 95% full-length recombinant spider silk protein.

43. The method of claim 41, wherein an amount of full-length recombinant spider silk protein is measured using a Western blot.

44. The method of claim 41, wherein an amount of full-length recombinant spider silk protein is measured using Size Exclusion Chromatography.

45. The method of any one of claims 1-44, wherein the purity of the isolated recombinant spider silk protein is 5-10%, 10-15%, 15-20%, 20-25%, 25-30%, 30-35%, 35-40%, 45-50%, 50-55%, 55-60%, 60-65%, 65-70%, 70-75%, 75-80%, 80-85%, 85-90%, 09-95%, or 95-100%.

46. The method of any one of claims 1-45, wherein the recombinant spider silk protein is a highly crystalline silk protein, a high beta sheet content silk protein, or a low solubility silk protein.

47. The method of any one of claims 1-46, wherein the recombinant spider silk protein comprises a sequence set forth in SEQ ID NOs: 1-27 or 39-59.

48. The method of any one of claims 1-47, wherein the cell culture comprises a fungal, a bacterial or a yeast cell.

49. The method of any one of claims 1-48, wherein the bacterial cell is Escherichia coli.

50. The method of any one of claims 1-49, further comprising drying the isolated recombinant spider silk protein to produce a silk protein powder.

51. A method of isolating a recombinant spider silk protein from a host cell, comprising:
providing a cell culture comprising a host cell, wherein the host cell expresses a recombinant spider silk protein;
collecting an insoluble portion derived from the cell culture, wherein the insoluble portion comprises the recombinant spider silk protein;
adding the insoluble portion of the host cell to a solution comprising at least 0.1M calcium chloride and methanol, thereby solubilizing the recombinant spider silk protein in the solution; and
isolating the recombinant spider silk protein from the solution, thereby producing an isolated recombinant spider silk protein.

52. The method of claim 51, wherein the solution comprises at least 1M, 1.5M, 2M, 2.5M, 3M, or 4M calcium chloride.

53. The method of claim 51, further comprising drying the isolated recombinant spider silk protein to produce a silk protein powder.

54. A composition comprising a recombinant spider silk protein produced by the method of any one of the above claims.

55. The composition of claim 54, comprising a recombinant spider silk protein powder.

56. The composition of claim 54 or 55, wherein the recombinant spider silk comprises at least 55%, at least 60%, at least 65%, at least 70%, at least 75%, at least 80%, at least 85%, at least 90%, at least 95%, or 100% full length recombinant spider silk.

57. A silk solid comprising a recombinant spider silk protein produced by the method of any one of claims 1-53.
